(19) United States
(12) Patent Application Publication
(10) Pub. No.: US 2006/0234237 A1
(43) Pub. Date: Oct. 19, 2006

(54) Biomarkers and methods for determining sensitivity to epidermal growth factor receptor modulators

(76) Inventors: Lukas C. Amler, Foster City, CA (US); Thomas Januario, San Francisco, CA (US)

Correspondence Address: LOUIS J. WILLE;BRISTOL-MYERS SQUIBB COMPANY, PATENT DEPARTMENT, P O BOX 4000, PRINCETON, NJ, 08543-4000, US

(21) Appl. No.: 10/541,749

(22) PCT Filed: Jan. 8, 2004
(86) PCT No.: PCT/US04/00368

§ 371 Date: Mar. 3, 2006

(51) Int. Cl. C12Q 1/68; G01N 33/574

(52) U.S. Cl. 435/6; 435/7.23

(57) ABSTRACT

EGFR biomakers useful in a method for identifying a mammal that will respond therapeutically to a method of treating cancer comprising administering an EGFR modulator, wherein the method comprises (a) exposing the mammal to the EGFR modulator and (b) measuring in the mammal level of at least one biomaker, wherein a difference in the level in at least one biomaker measured in (b) compared to the level of the biomaker in a mammal that has not been exposed to the EGFR modulator indicates that the mammal will respond therapeutically to the method of treating cancer.

### FIELD OF THE INVENTION

The present invention relates generally to the field of pharmacogenomics, and more specifically to methods and procedures to determine sensitivity in patients to allow the development of individualized genetic profiles which aid in treating diseases and disorders based on patient response at a molecular level.

### BACKGROUND OF THE INVENTION

Cancer is a disease with extensive histoclinical heterogeneity. Although conventional histological and clinical features have been correlated to prognosis, the same apparent prognostic type of tumors varies widely in its responsiveness to therapy and consequent survival of the patient.

New prognostic and predictive markers, which would facilitate an individualization of therapy for each patient, are needed to accurately predict patient response to treatments, such as small molecule or biological molecule drugs, in the clinic. The problem may be solved by the identification of new parameters that could better predict the patient's sensitivity to treatment. The classification of patient samples is a crucial aspect of cancer diagnosis and treatment. The association of a patient's response to a treatment with molecular and genetic markers can open up new opportunities for treatment development in non-responding patients, or distinguish a treatment's indication among other treatment choices because of higher confidence in the efficacy. Further, the pre-selection of patients who are likely to respond well to a medicine, drug, or combination therapy may reduce the number of patients needed in a clinical study or accelerate the time needed to complete a clinical development program (M. Cockett et al., 2000, Current Opinion in Biotechnology, 11:602-609).

The ability to predict drug sensitivity in patients is particularly challenging because drug responses reflect not only properties intrinsic to the target cells, but also a host's metabolic properties. Efforts to use genetic information to predict drug sensitivity have primarily focused on individual genes that have broad effects, such as the multidrug resistance genes, mdr1 and mrp1 (P. Sonneveld, 2000, J. Intern. Med., 247:521-534).

The development of microarray technologies for large scale characterization of gene mRNA expression pattern has made it possible to systematically search for molecular markers and to categorize cancers into distinct subgroups not evident by traditional histopathological methods (J. Khan et al., 1998, Cancer Res., 58:5009-5013; A. A. Alizadeh et al., 2000, Nature, 403:503-511; M. Bittner et al., 2000, Nature, 406:536-540; J. Khan et al., 2001, Nature Medicine, 7(6):673-679; and T. R. Golub et al., 1999, Science, 286:531-537; U. Alon et al., 1999, Proc. Natl. Acad. Sci. USA, 96:6745-6750). Such technologies and molecular tools have made it possible to monitor the expression level of a large number of transcripts within a cell population at any given time (see, e.g., Schena et al., 1995, Science, 270:467-470; Lockhart et al., 1996, Nature Biotechnology, 14:1675-1680; Blanchard et al., 1996, Nature Biotechnology, 14:1649; U.S. Pat. No. 5,569,588 to Ashby et al.).

Recent studies demonstrate that gene expression information generated by microarray analysis of human tumors can predict clinical outcome (L. J. van't Veer et al., 2002, Nature, 415:530-536; M. West et al., 2001, Proc. Natl. Acad. Sci. USA, 98:11462-11467; T. Sorlie et al., 2001, Proc. Natl. Acad. Sci USA, 98:10869-10874; M. Shipp et al., 2002, Nature Medicine, 8(1):68-74). These findings bring hope that cancer treatment will be vastly improved by better predicting the response of individual tumors to therapy.

Needed are new and alternative methods and procedures to determine drug sensitivity in patients to allow the development of individualized genetic profiles which are necessary to treat diseases and disorders based on patient response at a molecular level.

### SUMMARY OF THE INVENTION

The invention provides methods and procedures for determining patient sensitivity to one or more Epidermal Growth Factor Receptor (EGFR) modulators. The invention also provides methods of determining or predicting whether an individual requiring therapy for a disease state such as cancer will or will not respond to treatment, prior to administration of the treatment, wherein the treatment comprises one or more EGFR modulators. The one or more EGFR modulators are compounds that can be selected from, for example, one or more EGFR specific ligands, one or more small molecule EGFR inhibitors, or one or more EGFR binding monoclonal antibodies.

In one aspect, the invention provides a method for identifying a mammal that will respond therapeutically to a method of treating cancer comprising administering an EGFR modulator, wherein the method comprises: (a) measuring in the mammal the level of at least one biomarker selected from the biomarkers of Table 4; (b) exposing the mammal to the EGFR modulator; (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

As used herein, respond therapeutically refers to the alleviation or abrogation of the cancer. This means that the life expectancy of an individual affected with the cancer will be increased or that one or more of the symptoms of the cancer will be reduced or ameliorated. The term encompasses a reduction in cancerous cell growth or tumor volume. Whether a mammal responds therapeutically can be measured by many methods well known in the art, such as PET imaging.

The at least one biomarker can also be selected from the biomarkers of Table 5. The mammal can be, for example, a human, rat, mouse, dog rabbit, pig sheep, cow, horse, cat, primate, or monkey.

The method of the invention can be, for example, an in vitro method and wherein the at least one biomarker is measured in at least one mammalian biological sample from the mammal. The biological sample can comprise, for example, at least one of whole fresh blood, peripheral blood mononuclear cells, frozen whole blood, fresh plasma, frozen plasma, urine, saliva, skin, hair follicle, or tumor tissue.

In another aspect, the invention provides a method for identifying a mammal that will respond therapeutically to a method of treating cancer comprising administering an EGFR modulator, wherein the method comprises: (a) exposing the mammal to the EGFR modulator; (b) following the exposing of step (a), measuring in the mammal the level of the at least one biomarker selected from the biomarkers of Table 4, wherein a difference in the level of the at least one biomarker measured in step (b), compared to the level of the biomarker in a mammal that has not been exposed to said EGFR modulator, indicates that the mammal will respond therapeutically to said method of treating cancer.

In yet another aspect, the invention provides a method for testing or predicting whether a mammal will respond therapeutically to a method of treating cancer comprising administering an EGFR modulator, wherein the method comprises: (a) measuring in the mammal the level of at least one biomarker selected from the biomarkers of Table 4; (b) exposing the mammal to the EGFR modulator; (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

In another aspect, the invention provides a method for determining whether a compound inhibits EGFR activity in a mammal, comprising: (a) exposing the mammal to the compound; and (b) following the exposing of step (a), measuring in the mammal the level of at least one biomarker selected from the biomarkers of Table 4, wherein a difference in the level of said biomarker measured in step (b), compared to the level of the biomarker in a mammal that has not been exposed to said compound, indicates that the compound inhibits EGFR activity in the mammal.

In yet another aspect, the invention provides a method for determining whether a mammal has been exposed to a compound that inhibits EGFR activity, comprising (a) exposing the mammal to the compound; and (b) following the exposing of step (a), measuring in the mammal the level of at least one biomarker selected from the biomarkers of Table 4, wherein a difference in the level of said biomarker measured in step (b), compared to the level of the biomarker in a mammal that has not been exposed to said compound, indicates that the mammal has been exposed to a compound that inhibits EGFR activity.

In another aspect, the invention provides a method for determining whether a mammal is responding to a compound that inhibits EGFR activity, comprising (a) exposing the mammal to the compound; and (b) following the exposing of step (a), measuring in the mammal the level of at least one biomarker selected from the biomarkers of Table 4, wherein a difference in the level of said biomarker measured in step (b), compared to the level of the biomarker in a mammal that has not been exposed to said compound, indicates that the mammal is responding to the compound that inhibits EGFR activity.

As used herein, “responding” encompasses responding by way of a biological and cellular response, as well as a clinical response (such as improved symptoms, a therapeutic effect, or an adverse event), in a mammal The invention also provides an isolated biomarker selected from the biomarkers of Table 4. The biomarkers of the invention comprise sequences selected from the nucleotide and amino acid sequences provided in Table 4 and the Sequence Listing, as well as fragments and variants thereof.

The invention also provides a biomarker set comprising two or more biomarkers selected from the biomarkers of Table 4.

The invention also provides kits for determining or predicting whether a patient would be susceptible or resistant to a treatment that comprises one or more EGFR modulators. The patient may have a cancer or tumor such as, for example, a colon cancer or tumor.

In one aspect, the kit comprises a suitable container that comprises one or more specialized microarrays of the invention, one or more EGFR modulators for use in testing cells from patient tissue specimens or patient samples, and instructions for use. The kit may further comprise reagents or materials for monitoring the expression of a biomarker set at the level of mRNA or protein.

In another aspect, the invention provides a kit comprising two or more biomarkers selected from the biomarkers of Table 4.

In yet another aspect, the invention provides a kit comprising at least one of an antibody and a nucleic acid for detecting the presence of at least one of the biomarkers selected from the biomarkers of Table 4. In one aspect, the kit further comprises instructions for determining whether or not a mammal will respond therapeutically to a method of treating cancer comprising administering a compound that inhibits EGFR activity. In another aspect, the instructions comprise the steps of (a) measuring in the mammal the level of at least one biomarker selected from the biomarkers of Table 4, (b) exposing the mammal to the compound, (c) following the exposing of step (b), measuring in the mammal the level of the at least one biomarker, wherein a difference in the level of the at least one biomarker measured in step (c) compared to the level of the at least one biomarker measured in step (a) indicates that the mammal will respond therapeutically to said method of treating cancer.

The invention also provides screening assays for determining if a patient will be susceptible or resistant to treatment with one or more EGFR modulators.

The invention also provides a method of monitoring the treatment of a patient having a disease treatable by one or more EGFR modulators.

The invention also provides individualized genetic profiles which are necessary to treat diseases and disorders-based on patient response at a molecular level.

The invention also provides specialized microarrays, e.g., oligonucleotide microarrays or cDNA microarrays, comprising one or more biomarkers having expression profiles that correlate with either sensitivity or resistance to one or more EGFR modulators. The invention also provides antibodies, including polyclonal or monoclonal, directed against one or more biomarkers of the invention.

The invention will be better understood upon a reading of the detailed description of the invention when considered in connection with the accompanying figures.

### BRIEF DESCRIPTION OF THE FIGURES

FIG. 1 illustrates a EGFR biomarker identification and prioritization strategy.

FIG. 2A illustrates the RT-PCR results for EGFR in thirty one colon cancer cell lines to identify cell lines which do not have significant mRNA expression of EGFR.

FIG. 2B illustrates the IC₅₀ profile for twenty two colon cancer cell lines with an EGFR inhibitor compound, and determination of sensitive and resistant cell lines.

### DETAILED DESCRIPTION OF THE INVENTION

The invention provides biomarkers that respond to the modulation of a specific signal transduction pathway and also correlate with EGFR modulator sensitivity or resistance. These biomarkers can be employed for predicting response to one or more EGFR modulators. In one aspect, the biomarkers of the invention are those provided in Table 4 and the Sequence Listing, including both polynucleotide and polypeptide sequences.

The biomarkers were determined by an in vitro assay employing microarray technology to monitor simultaneously the expression pattern of thousands of discrete genes in untreated cells, whose response to the modulation of a signal transduction pathway, in particular the EGFR pathway, was tested on untreated cells whose sensitivity to EGFR modulators was tested. The biomarkers have expression levels in the cells that are dependent on the activity of the EFGR signal transduction pathway and that are also highly correlated with EGFR modulator sensitivity exhibited by the cells. Biomarkers serve as useful molecular tools for predicting a response to EGFR modulators, preferably biological molecules, small molecules, and the like that affect EGFR kinase activity via direct or indirect inhibition or antagonism of EGFR kinase function or activity.

EGFR Modulators

As used herein, the term “EGFR modulator” is intended to mean a compound or drug that is a biological molecule or a small molecule that directly or indirectly modulates EGFR activity or the EGFR signal transduction pathway. Thus, compounds or drugs as used herein is intended to include both small molecules and biological molecules. Direct or indirect modulation includes activation or inhibition of EGFR activity or the EGFR signal transduction pathway. In one aspect, inhibition refers to inhibition of the binding of EGFR to an EGFR ligand such as, for example, EGF. In another aspect, inhibition refers to inhibition of the kinase activity of EGFR.

EGFR modulators include, for example, EGFR specific ligands, small molecule EGFR inhibitors, and EGFR monoclonal antibodies. In one aspect, the EGFR modulator inhibits EGFR activity and/or inhibits the EGFR signal transduction pathway. In another aspect, the EGFR modulator is an EGFR antibody that inhibits EGFR activity and/or inhibits the EGFR signal transduction pathway.

EGFR modulators include biological molecules or small molecules. Biological molecules include all lipids and polymers of monosaccharides, amino acids, and nucleotides having a molecular weight greater than 450. Thus, biological molecules include, for example, oligosaccharides and polysaccharides; oligopeptides, polypeptides, peptides, and proteins; and oligonucleotides and polynucleotides. Oligonucleotides and polynucleotides include, for example, DNA and RNA.

Biological molecules further include derivatives of any of the molecules described above. For example, derivatives of biological molecules include lipid and glycosylation derivatives of oligopeptides, polypeptides, peptides, and proteins.

Derivatives of biological molecules further include lipid derivatives of oligosaccharides and polysaccharides, e.g., lipopolysaccharides. Most typically, biological molecules are antibodies, or functional equivalents of antibodies. Functional equivalents of antibodies have binding characteristics comparable to those of antibodies, and inhibit the growth of cells that express EGFR. Such functional equivalents include, for example, chimerized, humanized, and single chain antibodies as well as fragments thereof.

Functional equivalents of antibodies also include polypeptides with amino acid sequences substantially the same as the amino acid sequence of the variable or hypervariable regions of the antibodies. An amino acid sequence that is substantially the same as another sequence, but that differs from the other sequence by means of one or more substitutions, additions, and/or deletions, is considered to be an equivalent sequence. Preferably, less than 50%, more preferably less than 25%, and still more preferably less than 10%, of the number of amino acid residues in a sequence are substituted for, added to, or deleted from the protein.

The functional equivalent of an antibody is preferably a chimerized or humanized antibody. A chimerized antibody comprises the variable region of a non-human antibody and the constant region of a human antibody. A humanized antibody comprises the hypervariable region (CDRs) of a non-human antibody. The variable region other than the hypervariable region, e.g., the framework variable region, and the constant region of a humanized antibody are those of a human antibody.

Suitable variable and hypervariable regions of non-human antibodies may be derived from antibodies produced by any non-human mammal in which monoclonal antibodies are made. Suitable examples of mammals other than humans include, for example, rabbits, rats, mice, horses, goats, or primates.

Functional equivalents further include fragments of antibodies that have binding characteristics that are the same as, or are comparable to, those of the whole antibody. Suitable fragments of the antibody include any fragment that comprises a sufficient portion of the hypervariable (i.e., complementarity determining) region to bind specifically, and with sufficient affinity, to EGFR tyrosine kinase to inhibit growth of cells that express such receptors.

Such fragments may, for example, contain one or both Fab fragments or the F(ab′)₂ fragment. Preferably, the antibody fragments contain all six complementarity determining regions of the whole antibody, although functional fragments containing fewer than all of such regions, such as three, four, or five CDRs, are also included.

In one aspect, the fragments are single chain antibodies, or Fv fragments. Single chain antibodies are polypeptides that comprise at least the variable region of the heavy chain of the antibody linked to the variable region of the light chain, with or without an interconnecting linker. Thus, Fv fragment comprises the entire antibody combining site. These chains may be produced in bacteria or in eukaryotic cells.

The antibodies and functional equivalents may be members of any class of immunoglobulins, such as IgG, IgM, IgA, IgD, or IgE, and the subclasses thereof. In one aspect, the antibodies are members of the IgG1 subclass. The functional equivalents may also be equivalents of combinations of any of the above classes and subclasses.

In one aspect, EGFR antibodies can be selected from chimerized, humanized, fully human, and single chain antibodies derived from the murine antibody 225 described in U.S. Pat. No. 4,943,533 to Mendelsohn et al. In one aspect, the 225 derived antibodies have the following hypervariable (CDR) regions of the light and heavy chain, wherein the amino acid sequences are indicated below the nucleotide sequences:

Heavy Chain Hypervarlible Regions (VH):

| | |
|---|---|
| CDR1 | |
| AACTATGGTGTACAC | (SEQ ID NO: 179) |
| N Y G V H | (SEQ ID NO: 180) |
| CDR2 | |
| GTGATATGGAGTGGTGGAAACACAGACTATAATACACCTTTCACATCC | (SEQ ID NO: 181) |
| V I W S G G N T D Y N T P F T S | (SEQ ID NO: 182) |
| CDR3 | |
| GCCCTCACCTACTATGATTACGAGTTTGCTTAC | (SEQ ID NO: 183) |
| A L T Y Y D Y E F A Y | (SEQ ID NO: 184) |
| LIGHT CHAIN HYPERVARIABLE REGIONS (VL): | |
| CDR1 | |
| AGGGCCAGTCAGAGTATTGGCACAAACATACAC | (SEQ ID NO: 185) |
| R A S Q S I G T N I H | (SEQ ID NO: 186) |
| CDR2 | |
| GCTTCTGAGTCTATCTCT | (SEQ ID NO: 187) |
| A S E S I S | (SEQ ID NO: 188) |
| CDR3 | |
| CAACAAAATAATAACTGGCCAACCACG | (SEQ ID NO: 189) |
| Q Q N N N W P T T | (SEQ ID NO: 190) |

In another aspect, the EGFR antibody can be selected from the antibodies described in U.S. Pat. No. 6,235,883 to Jakobovits et al., U.S. Pat. No. 5,558,864 to Bendi et al., and U.S. Pat. No. 5,891,996 to Mateo de Acosta del Rio et al.

In addition to the biological molecules discussed above, the EGFR modulators useful in the invention may also be small molecules. Any molecule that is not a biological molecule is considered herein to be a small molecule. Some examples of small molecules include organic compounds, organometallic compounds, salts of organic and organometallic compounds, saccharides, amino acids, and nucleotides. Small molecules further include molecules that would otherwise be considered biological molecules, except their molecular weight is not greater than 450. Thus, small molecules may be lipids, oligosaccharides, oligopeptides, and oligonucleotides and their derivatives, having a molecular weight of 450 or less.

It is emphasized that small molecules can have any molecular weight. They are merely called small molecules because they typically have molecular weights less than 450. Small molecules include compounds that are found in nature as well as synthetic compounds. In one embodiment, the EGFR modulator is a small molecule that inhibits the growth of tumor cells that express EGFR. In another embodiment, the EGFR modulator is a small molecule that inhibits the growth of refractory tumor cells that express EGFR.

Numerous small molecules have been described as being useful to inhibit EGFR. For example, U.S. Pat. No. 5,656,655 to Spada et al. discloses styryl substituted heteroaryl compounds that inhibit EGFR. The heteroaryl group is a monocyclic ring with one or two heteroatoms, or a bicyclic ring with 1 to about 4 heteroatoms, the compound being optionally substituted or polysubstituted.

U.S. Pat. No. 5,646,153 to Spada et al. discloses bis mono and/or bicyclic aryl heteroaryl, carbocyclic, and heterocarbocyclic compounds that inhibit EGFR.

U.S. Pat. No. 5,679,683 to Bridges et al. discloses tricyclic pyrimidine compounds that inhibit the EGFR. The compounds are fused heterocyclic pyrimidine derivatives described at column 3, line 35 to column 5, line 6.

U.S. Pat. No. 5,616,582 to Barker discloses quinazoline derivatives that have receptor tyrosine kinase inhibitory activity.

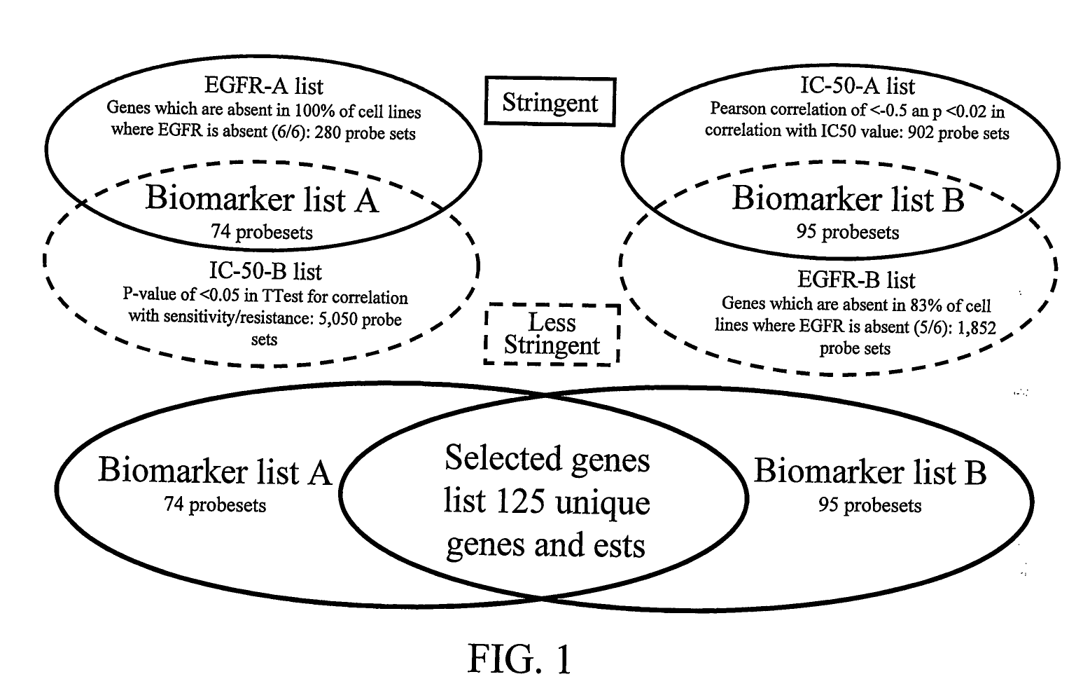

Fry et al., Science 265, 1093-1095 (1994) in FIG. 1 discloses a compound having a structure that inhibits EGFR.

Osherov et al. disclose tyrphostins that inhibit EGFR/HER1 and HER 2, particularly those in Tables I, II, III, and IV.

U.S. Pat. No. 5,196,446 to Levitzki et al. discloses heteroarylethenediyl or heteroarylethendeiylaryl compounds that inhibit EGFR, particularly from column 2, line 42 to column 3, line 40.

Panek et al., Journal of Pharmacology and Experimental Therapeutics 283, 1433-1444 (1997) discloses a compound identified as PD166285 that inhibits the EGFR, PDGFR, and FGFR families of receptors. PD166285 is identified as 6-(2,6-dichlorophenyl)-2-(4-(2-diethylaminoethyoxy)phenylamino)-8-methyl-8H-pyrido(2,3-d)pyrimidin-7-one having the structure shown in FIG. 1 on page 1436.

Biomarkers and Biomarker Sets

The invention includes individual biomarkers and biomarker sets having both diagnostic and prognostic value in disease areas in which signaling through EGFR or the EGFR pathway is of importance, e.g., in cancers or tumors, in immunological disorders, conditions or dysfunction, or in disease states in which cell signaling and/or cellular proliferation controls are abnormal or aberrant. The biomarker sets comprise a plurality of biomarkers such as, for example, a plurality of the biomarkers provided in Table 4 below, that highly correlate with resistance or sensitivity to one or more EGFR modulators.

The biomarker sets of the invention enable one to predict or reasonably foretell the likely effect of one or more EGFR modulators in different biological systems or for cellular responses. The biomarker sets can be used in in vitro assays of EGFR modulator response by test cells to predict in vivo outcome. In accordance with the invention, the various biomarker sets described herein, or the combination of these biomarker sets with other biomarkers or markers, can be used, for example, to predict how patients with cancer might respond to therapeutic intervention with one or more EGFR modulators.

A biomarker set of cellular gene expression patterns correlating with sensitivity or resistance of cells following exposure of the cells to one or more EGFR modulators provides a useful tool for screening one or tumor samples before treatment with the EGFR modulator. The screening allows a prediction of cells of a tumor sample exposed to one or more EGFR modulators, based on the expression results of the biomarker set, as to whether or not the tumor, and hence a patient harboring the tumor, will or will not respond to treatment with the EGFR modulator.

The biomarker or biomarker set can also be used as described herein for monitoring the progress of disease treatment or therapy in those patients undergoing treatment for a disease involving an EGFR modulator.

The biomarkers serve as targets for the development of therapies for disease treatment Such targets may be particularly applicable to treatment of breast disease, such as breast cancers or tumors. Indeed, because these biomarkers are differentially expressed in sensitive and resistant cells, their expression patterns are correlated with relative intrinsic sensitivity of cells to treatment with EGFR modulators. Accordingly, the biomarkers highly expressed in resistant cells may serve as targets for the development of new therapies for the tumors which are resistant to EGFR modulators, particularly EGFR inhibitors.

Microarrays

The invention also includes specialized microarrays, e.g., oligonucleotide microarrays or cDNA microarrays, comprising one or more biomarkers, showing expression profiles that correlate with either sensitivity or resistance to one or more EGFR modulators. Such microarrays can be employed in in vitro assays for assessing the expression level of the biomarkers in the test cells from tumor biopsies, and determining whether these test cells are likely to be resistant or sensitive to EGFR modulators. For example, a specialized microarray can be prepared using all the biomarkers, or subsets thereof, as described herein and shown in Table 4. Cells from a tissue or organ biopsy can be isolated and exposed to one or more of the EGFR modulators. Following application of nucleic acids isolated from both untreated and treated cells to one or more of the specialized microarrays, the pattern of gene expression of the tested cells can be determined and compared with that of the biomarker pattern from the control panel of cells used to create the biomarker set on the microarray. Based upon the gene expression pattern results from the cells that underwent testing, it can be determined if the cells show a resistant or a sensitive profile of gene expression. Whether or not the tested cells from a tissue or organ biopsy will respond to one or more of the EGFR modulators and the course of treatment or therapy can then be determined or evaluated based on the information gleaned from the results of the specialized microarray analysis.

Antibodies

The invention also includes antibodies, including polyclonal or monoclonal, directed against one or more of the polypeptide biomarkers. Such antibodies can be used in a variety of ways, for example, to purify, detect, and target the biomarkers of the invention, including both in vitro and in vivo diagnostic, detection, screening, and/or therapeutic methods.

Kits

The invention also includes kits for determining or predicting whether a patient would be susceptible or resistant to a treatment that comprises one or more EGFR modulators. The patient may have a cancer or tumor such as, for example, a breast cancer or tumor. Such kits would be useful in a clinical setting for use in testing a patient's biopsied tumor or cancer samples, for example, to determine or predict if the patient's tumor or cancer will be resistant or sensitive to a given treatment or therapy with an EGFR modulator. The kit comprises a suitable container that comprises: one or more microarrays, e.g., oligonucleotide microarrays or cDNA microarrays, that comprise those biomarkers that correlate with resistance and sensitivity to EGFR modulators, particularly EGFR inhibitors; one or more EGFR modulators for use in testing cells from patient tissue specimens or patient samples; and instructions for use. In addition, kits contemplated by the invention can further include, for example, reagents or materials for monitoring the expression of biomarkers of the invention at the level of mRNA or protein, using other techniques and systems practiced in the art such as, for example, RT-PCR assays, which employ primers designed on the basis of one or more of the biomarkers described herein, immunoassays, such as enzyme linked immunosorbent assays (ELISAs), immunoblotting, e.g., Western blots, or in situ hybridization, and the like, as further described herein.

Application of Biomarkers and Biomarker Sets

The biomarkers and biomarker sets may be used in different applications. Biomarker sets can be built from any combination of biomarkers listed in Table 4 to make predictions about the likely effect of any EGFR modulator in different biological systems. The various biomarkers and biomarker sets described herein can be used, for example, as diagnostic or prognostic indicators in disease management, to predict how patients with cancer might respond to therapeutic intervention with compounds that modulate the EGFR, and to predict how patients might respond to therapeutic intervention that modulates signaling through the entire EGFR regulatory pathway.

While the data described herein were generated in cell lines that are routinely used to screen and identify compounds that have potential utility for cancer therapy, the biomarkers have both diagnostic and prognostic value in other diseases areas in which signaling through EGFR or the EGFR pathway is of importance, e.g., in immunology, or in cancers or tumors in which cell signaling and/or proliferation controls have gone awry.

In the examples described below, the sensitivity and resistance classifications in the twenty two colon cell lines were similar for the two EGFR modulators tested. Therefore, the biomarkers of the invention are expected to have both diagnostic and prognostic value for other compounds that modulate EGFR or the EGFR signaling pathways.

Those having skill in the pertinent art will appreciate that the EGFR signaling pathway is used and functional in cell types other than cell lines of colon tissue. Therefore, the described biomarkers are expected to have utility for predicting drug sensitivity or resistance to compounds that interact with or inhibit the EGFR activity in cells from other tissues or organs associated with a disease state, or cancers or tumors derived from other tissue types. Non-limiting examples of such cells, tissues and organs include breast, colon, lung, prostate, testes, ovaries, cervix, esophagus, pancreas, spleen, liver, kidney, stomach, lymphocytic and brain, thereby providing a broad and advantageous applicability to the biomarkers described herein. Cells for analysis can be obtained by conventional procedures as known in the art, for example, tissue biopsy, aspiration, sloughed cells, e.g., colonocytes, clinical or medical tissue or cell sampling procedures.

In accordance with the invention, cells from a patient tissue sample, e.g., a tumor or cancer biopsy, can be assayed to determine the expression pattern of one or more biomarkers prior to treatment with one or more EGFR modulators. Success or failure of a treatment can be determined based on the biomarker expression pattern of the cells from the test tissue (test cells), e.g., tumor or cancer biopsy, as being relatively similar or different from the expression pattern of a control set of the one or more biomarkers. Thus, if the test cells show a biomarker expression profile which corresponds to that of the biomarkers in the control panel of cells which are sensitive to the EGFR modulator, it is highly likely or predicted that the individual's cancer or tumor will respond favorably to treatment with the EGFR modulator. By contrast, if the test cells show a biomarker expression pattern corresponding to that of the biomarkers of the control panel of cells which are resistant to the EGFR modulator, it is highly likely or predicted that the individual's cancer or tumor will not respond to treatment with the EGFR modulator.

The invention also provides a method of monitoring the treatment of a patient having a disease treatable by one or more EGFR modulators. The isolated test cells from the patient's tissue sample, e.g., a tumor biopsy or tumor sample, can be assayed to determine the expression pattern of one or more biomarkers before and after exposure to an EGFR modulator wherein, preferably, the EGFR modulator is an EGFR inhibitor. The resulting biomarker expression profile of the test cells before and after treatment is compared with that of one or more biomarkers as described and shown herein to be highly expressed in the control panel of cells that are either resistant or sensitive to an EGFR modulator. Thus, if a patient's response is sensitive to treatment by an EGFR modulator, based on correlation of the expression profile of the one or biomarkers, the patient's treatment prognosis can be qualified as favorable and treatment can continue. Also, if, after treatment with an EGFR modulator, the test cells don't show a change in the biomarker expression profile corresponding to the control panel of cells that are sensitive to the EGFR modulator, it can serve as an indicator that the current treatment should be modified, changed, or even discontinued. This monitoring process can indicate success or failure of a patient's treatment with an EGFR modulator and such monitoring processes can be repeated as necessary or desired.

The biomarkers of the invention can be used to predict an outcome prior to having any knowledge about a biological system. Essentially, a biomarker can be considered to be a statistical tool. Biomarkers are useful primarily in predicting the phenotype that is used to classify the biological system. In an embodiment of the invention, the goal of the prediction is to classify cancer cells as having an active or inactive EGFR pathway. Cancer cells with an inactive EGFR pathway can be considered resistant to treatment with an EGFR modulator. An inactive EGFR pathway is defined herein as a non-significant expression of the EGFR or by a classification as “resistant” or “sensitive” based on the IC₅₀ value of each colon cell line to a compound (EGFR inhibitor compound BMS-461453) exemplified herein.

A number of the biomarker described herein are known to be regulated by EGFR, e.g., mucin 2 (J Biol Chem. 2002 Aug. 30; 277(35):32258-67). Another biomarker, betacellulin, is know to be an EGFR ligand (Biochem Biophys Res Commun. 2002 Jun. 28; 294(5):1040-6). A functional relationship of the top biomarkers to the EGFR is expected, since biomarkers that contribute to high biomarker accuracy are likely to play a functional role in the pathway that is being modulated. For example, Perception therapy (i.e., antibody that binds to the Her2 receptor and prevents function via internalization) is indicated when the Her2 gene is overexpressed. It is unlikely that a therapy will have any therapeutic effect if the target enzyme is not expressed.

However, although the complete function of all of the biomarkers are not currently known, some of the biomarkers are likely to be directly or indirectly involved in the EGFR signaling pathway. In addition, some of the biomarkers may function in the metabolic or other resistance pathways specific to the EGFR modulators tested. Notwithstanding, knowledge about the function of the biomarkers is not a requisite for determining the accuracy of a biomarker according to the practice of the invention.

Discovery of Biomarkers

An approach has been discovered in which biomarkers were identified whose expression patterns, in a subset of cell lines, correlated to and can be used as an in vitro marker of cellular response to treatment or therapy with one compound, or with a combination or series of compounds, that are known to inhibit or activate the function of a protein, enzyme, or molecule (e.g., a receptor) that is directly or indirectly involved in cell proliferation, cell responses to external stimuli, (such as ligand binding), or signal transduction, e.g., a receptor tyrosine kinase. Preferred are antagonists or inhibitors of the function of a given protein, e.g., a receptor tyrosine kinase.

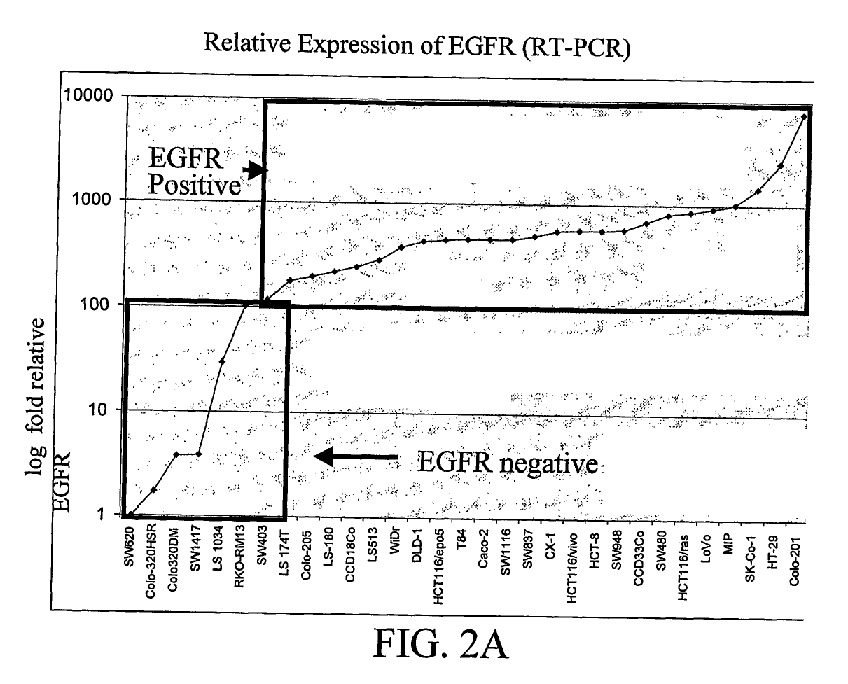

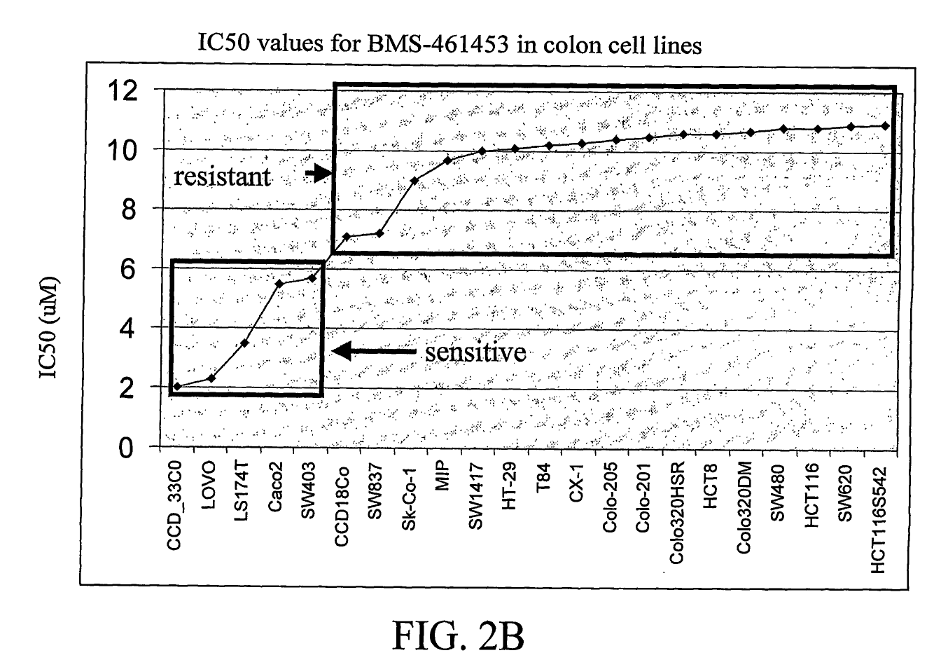

Two analytical strategies were deployed to discover biomarkers useful for predicting the sensitivity or resistance of cancer cells to treatment with one or more EGFR modulators. FIG. 1 illustrates the EGFR biomarker identification and prioritization strategy. In one strategy, the mRNA expression level of EGFR was used to identify six colon cancer cell lines with, inferred from the mRNA expression level, no significant presence of the EGFR protein and hence no significant activity of the EGFR pathway (FIG. 2A). In subsequent analyses, biomarkers were identified that had no significant mRNA expression level in the six cell lines and no inferred presence of the EGFR protein. Further, it was required that these biomarkers would have a significant mRNA expression level in at least six other cell lines.

In a second strategy, an EGFR specific tyrosine kinase inhibitor compound was used to determine compound sensitivity in a panel of twenty two colon cancer cell lines following exposure of the cells to the compound. Some of the cell lines were determined to be resistant to treatment with the inhibitor compound, while others were determined to be sensitive to the inhibitor (FIG. 2B). A subset of the cell lines examined provided an expression pattern or profile of biomarkers that correlated to a response by the cells to the EGFR inhibitor compound as well as the absence of significant EGFR expression as thus could serve as biomarkers.

By combining the use of EGFR co-regulation studies in tumor cells with experimental studies in cultured cells as a model of in vivo effects, the invention advantageously focuses on cell-intrinsic properties that are exposed in cell culture to identify biomarkers that predict compound sensitivity and resistance. The discovery and identification of biomarkers in tumor cells and cell lines assayed in vitro can be used to predict responses to one or more EGFR modulators in vivo and, thus, can be extended to clinical situations in which the same biomarkers are used to predict patients' responses to one or more EGFR modulators and treatments comprising one or more EGFR modulators.

As described in the examples below, oligonucleotide microarrays were used to measure the expression levels of over 44,792 probe sets in a panel of thirty one untreated colon cancer cell lines for which the expression status of the EGFR and the drug sensitivity to EGFR inhibitor compounds was determined. This analysis was performed to determine whether the gene expression signatures of untreated cells were sufficient for the prediction of sensitivity of the disease to inhibition of the EGFR by small molecule or biological molecule compounds. Through data analysis, biomarkers were identified whose expression levels were found to be highly counter-correlated with the status of the EGFR and correlated with the drug sensitivity. In addition, the treatment of cells with a small molecule EGFR inhibitor also provided gene expression signatures predictive of sensitivity to the compound.

The means of performing the gene expression and biomarker identification analyses embraced by the invention is described in further detail and without limitation below.

IC₅₀ Determination and Phenotype Classification Based on Sensitivity of Twenty-Two Colon Cancer Cell Lines to EGFR Inhibitor Compounds

Twenty two colon cell lines were treated with a small molecule EGFR inhibitor (BMS-461453) to determine the individual IC₅₀ value. The IC₅₀ for each cell line was assessed by MTS assays. The average IC₅₀ values along with standard deviations were calculated from two to five individual determinations for each cell line. As shown in FIG. 2B, a 4-fold variation in the IC₅₀ values was observed for the small molecule EGFR inhibitor among the 22 colon cancer cell lines. The IC₅₀ unit is μM.

All cell lines with at least a 1.75 fold lower IC₅₀ than the most resistant cell lines were considered to be sensitive to treatment with the small molecule EGFR inhibitor. FIG. 2B represents the resistance/sensitivity classifications of the twenty-two colon cell lines to the small molecule EGFR inhibitor. Five cell lines were classified as sensitive and seventeen cell lines as resistant.

Description of the Strategy for Identifying Biomarkers

Biomarkers were discovered based on two criteria: (i) the correlation of their mRNA expression level to the expression of EGFR in cell lines with insignificant EGFR expression and (ii) the correlation of the IC₅₀ values for the small molecule EGFR inhibitor BMS-461453 with gene expression levels.

For each of these two biomarker selection strategies, two independent “discovery” probe set lists were established by using statistical filters with different stringency levels to identify genes whose expression correlated with either EGFR status or IC₅₀ value. These statistical methods are described below and resulted in four discovery probe set lists: EGFR-A and EGFR-B (correlation with no significant EGFR expression) and IC-50-A, IC-50-B (correlation with IC₅₀ expression), the A-lists containing probe sets selected by more stringent conditions. To then establish two biomarker probe set lists, probe sets that appeared in both EGFR-A and IC-50 B were selected (Biomarker Probe Set List A, Table 2) and probe sets that appeared in both EGFR-B and IC-50-A were selected (Biomarker Probe Set List B, Table 3).

Identifying Genes that Significantly Correlate with EGFR Status Classification

RT-PCR expression data for EGFR were obtained from thirty one colon cancer cell lines and six cell lines with a significantly lower expression level of EGFR compared to the other cell lines were identified as described in Example 1 below. (FIG. 2A). Expression profiling data of 44,792 probe sets represented on the HG-U133 array set for all Dirty one untreated colon cancer cell lines were obtained and analyzed for the identification of probe sets which would be correlated with the above described six cell lines with no significant mRNA expression of EGFR. For the discovery probe set list EGFR-A, all probe sets which were judged to be absent by the Affymetrix Mas 5.0 software in six of the six colon cancer cell lines with significantly lower expression of EGFR were identified. Second, it was required that these probe sets would be judged to be present in at least six cell lines of the twenty five cell lines classified as having significant mRNA expression of the EGFR This analytical strategy resulted in the identification of 280 probe sets that could be analyzed in comparison to the discovery probe set list IC-50-B.

The discovery probe set list EGFR-B was generated by selecting all probe sets which were judged to be absent by the Affymetrix Mas 5.0 software in five of the six colon cancer cell lines with significantly lower expression of EGFR and which would be present in at least six cell lines of the twenty five cell lines classified as having significant mRNA expression of the EGFR. Discovery probe set list EGR-B contains 1,852 probe sets (1133A: 876; U133B: 976).

Identifying Genes that Significantly Correlate with Drug Resistance/Sensitivity Classification

Expression profiling data of 44,792 probe sets represented on the HG-U133 array set for twenty two untreated colon cell lines were obtained and preprocessed as described in Example 1 below. These data were analyzed using the Student's TTEST to identify genes whose expression patterns were strongly correlated with the drug resistance/sensitivity classification. Table 1 provides the resistance/sensitivity phenotype classification of the twenty two colon cell lines for the EGFR antagonist BMS-461453 based on the IC₅₀ results. The mean IC₅₀ values along with standard deviations (SD) were calculated from 2 to 5 individual determinations for each cell line as shown. The mean IC₅₀ across the twenty two colon cell lines for BMS-461453 was calculated and used to normalize the IC₅₀ data for each cell line. All cell lines with at least a 1.75 fold lower IC₅₀ than the most resistant cell lines were considered to be sensitive to treatment with BMS-5461453. The cell lines designated with an asterisk are defined as being sensitive to the drug treatment.

TABLE 1
Resistance/Sensitivity Phenotype Classification of Twenty Two Colon
Cell Lines

| Cell lines | IC₅₀ (μM) | SD |
|---|---|---|
| CCD_33C0* | 2 | 1.28 |
| LOVO* | 2.3 | 2.28 |
| LS174T* | 3.5 | 1.93 |
| Caco2* | 5.5 | 3.97 |
| SW403* | 5.7 | 4.94 |
| CCD18Co | 7.1 | 3.84 |
| SW837 | 7.2 | 3.30 |
| Sk-Co-1 | 9 | 2.02 |
| MIP | 9.7 | 0.52 |
| SW1417 | 10 | 0.00 |
| HT-29 | 10 | 0.00 |
| T84 | 10 | 0.00 |
| CX-1 | 10 | 0.00 |
| Colo-205 | 10 | 0.00 |
| Colo-201 | 10 | 0.00 |
| Colo320HSR | 10 | 0.00 |
| HCT8 | 10 | 0.00 |
| Colo320DM | 10 | 0.00 |
| SW480 | 10 | 0.00 |
| HCT116 | 10 | 0.00 |
| SW620 | 10 | 0.00 |
| HCT116S542 | 10 | 0.00 |

An “idealized expression pattern” corresponds to a gene that is uniformly high in one class (e.g., sensitive) and uniformly low in the other (e.g., resistant). Initially, a Student TTEST was performed in which a T value was obtained for each probe set. Once a T value was generated, its corresponding confidence value (P) was found on a standard table of significance. The confidence value is a measure of the probability to observe a certain mean expression difference between two groups by chance alone and is obtained using the following formula:
T(g·c)=(X₁−X₂)/(var₁/n₁+var₂/n₂)^{1/2}
wherein,
T(g,c) represents the T value between expression for gene g and the sensitivity/resistance classification c;
X₁ represents mean gene expression level of samples in class 1;
X₂ represents mean gene expression level of samples in class 2;
var₁ represents variance of gene expression for samples in class 1;
var₂ represents variance of gene expression for samples in class 2;
n₁ represents number of samples in class 1;
n₂ represents number of samples in class 2; and
corresponding confidence value (P) for T values are obtained from a standard table of significance.

To generate discovery probe set list IC-50-B, a confidence value of 0.05 or lower was used as the cut off for probe sets to be included in the list. Discovery probe set list IC-50-B contains 5,050 probe sets (U133A: 2,498; U133B: 2,552).

Discovery probe set list IC-50-A was generated using the Pearson correlation coefficient (a dimensionless index that ranges from −1.0 to 1.0). This value was calculated by treating the IC₅₀ data as continuous variables and by utilizing a linear regression model to correlate gene expression levels with IC₅₀ values for twenty-two colon cell lines. Probe sets with a correlation coefficient less than −0.5 were selected (p<0.02), a total of 902 probe sets (U133A: 467; U133B: 435).

Finally, two separate biomarker probe set lists were generated, biomarker probe set lists A and B, by identifying probe sets which were present in EGFR-A and IC-50-B (Biomarker Probe Set List A) (Table 2) or were present in EGFR-B and IC-50-A (Biomarker Probe Set List B) (Table 3).

The biomarker probe set list A (Table 2) contains a total of 74 probe sets (U133A: 43; U133B: 31) and provides the polynucleotides identified to be biomarkers of EGFR antagonist sensitivity employing strategy A. With strategy A, polynucleotides were required to satisfy a stringent criteria for EGFR status coregulation and a less stringent condition for correlation to IC₅₀ values. Namely, the polynucleotides had to be called absent by the Affymetrix software in six out of the six cell lines with lowest expression of EGFR and be differentially expressed in the sensitive and resistance cell lines with a P value equal to or less than 0.05.

TABLE 2
Biomarker Probe Set List A

| | | Affymetrix |
|---|---|---|
| Unigene Title | Affymetrix Description | probe set |
| hemoglobin, | gb: BC005931.1 /DEF = Homo sapiens, | 211745_x_at |
| alpha 1 | hemoglobin, alpha 2, clone MGC: 14541, mRNA, | |
| | complete cds. /FEA = mRNA | |
| | /PROD = hemoglobin, alpha 2 | |
| | /DB_XREF = gi: 13543547 /FL = gb: BC005931.1 | |
| dipeptidylpeptidase | gb: M80536.1 /DEF = H. sapiens dipeptidylpeptidase | 203716_s_at |
| IV (CD26, | IV (DPP4) mRNA, complete cds. | |
| adenosine | /FEA = mRNA /GEN = DPP4 /PROD = dipeptidylpeptidase | |
| deaminase | IV /DB_XREF = gi: 181569 | |
| complexing | /UG = Hs.44926 dipeptidylpeptidase IV (CD26, | |
| protein 2) | adenosine deaminase complexing protein 2) | |
| | /FL = gb: M80536.1 gb: NM_001935.1 | |
| spondin 1, (f- | Consensus includes gb: AI885290 /FEA = EST | 213994_s_at |
| spondin) | /DB_XREF = gi: 5590454 | |
| extracellular | /DB_XREF = est: wl92a04.x1 | |
| matrix protein | /CLONE = IMAGE: 2432334 /UG = Hs.5378 | |
| | spondin 1, (f-spondin) extracellular matrix | |
| | protein | |
| 3-hydroxy-3- | gb: NM_005518.1 /DEF = Homo sapiens 3- | 204607_at |
| methylglutaryl- | hydroxy-3-methylglutaryl-Coenzyme A synthase | |
| Coenzyme A | 2 (mitochondrial) (HMGCS2), mRNA. | |
| synthase 2 | /FEA = mRNA /GEN = HMGCS2 /PROD = 3- | |
| (mitochondrial) | hydroxy-3-methylglutaryl-Coenzyme A synthase | |
| | 2(mitochondrial) /DB_XREF = gi: 5031750 | |
| | /UG = Hs.59889 3-hydroxy-3-methylglutaryl- | |
| | Coenzyme A synthase 2 (mitochondrial) | |
| | /FL = gb: NM_005518.1 | |
| mucin 2, | gb: NM_002457.1 /DEF = Homo sapiens mucin 2, | 204673_at |
| intestinal/trachea 1 | intestinaltracheal (MUC2), mRNA. /FEA = mRNA | |
| | /GEN = MUC2 /PROD = mucin 2, intestinaltracheal | |
| | /DB_XREF = gi: 4505284 /UG = Hs.315 mucin 2, | |
| | intestinaltracheal /FL = gb: NM_002457.1 | |
| | gb: L21998.1 | |
| cystic fibrosis | gb: NM_000492.2 /DEF = Homo sapiens cystic | 205043_at |
| transmembrane | fibrosis transmembrane conductance regulator, | |
| conductance | ATP-binding cassette (sub-family C, member 7) | |
| regulator, ATP- | (CFTR), mRNA. /FEA = mRNA /GEN = CFTR | |
| binding cassette | /PROD = cystic fibrosis transmembrane | |
| (sub-family C, | conductanceregulator, ATP-binding cassette (sub- | |
| member 7) | family C, member 7) /DB_XREF = gi: 6995995 | |
| | /UG = Hs.663 cystic fibrosis transmembrane | |
| | conductance regulator, ATP-binding cassette | |
| | (sub-family C, member 7) /FL = gb: NM_000492.2 | |
| CUG triplet | Consensus includes gb: N36839 /FEA = EST | 202156_s_at |
| repeat, RNA- | /DB_XREF = gi: 1157981 | |
| binding protein 2 | /DB_XREF = est: yy35f07.s1 | |
| | /CLONE = IMAGE: 273253 /UG = Hs.211610 CUG | |
| | triplet repeat, RNA-binding protein 2 | |
| | /FL = gb: U69546.1 gb: AF036956.1 | |
| | gb: AF090694.1 gb: NM_006561.1 | |
| nuclear receptor | gb: NM_000901.1 /DEF = Homo sapiens nuclear | 205259_at |
| subfamily 3, | receptor subfamily 3, group C, member 2 | |
| group C, member 2 | (NR3C2), mRNA. /FEA = mRNA /GEN = NR3C2 | |
| | /PROD = nuclear receptor subfamily 3, group C, | |
| | member 2 /DB_XREF = gi: 4505198 /UG = Hs.1790 | |
| | nuclear receptor subfamily 3, group C, member 2 | |
| | /FL = gb: M16801.1 gb: NM_000901.1 | |
| cystic fibrosis | Consensus includes gb: W60595 /FEA = EST | 215702_s_at |
| transmembrane | /DB_XREF = gi: 1367354 | |
| conductance | /DB_XREF = est: zc91b04.s1 | |
| regulator, ATP- | /CLONE = IMAGE: 338479 /UG = Hs.663 cystic | |
| binding cassette | fibrosis transmembrane conductance regulator, | |
| (sub-family C, | ATP-binding cassette (sub-family C, member 7) | |
| member 7) | | |
| cytochrome | gb: NM_000775.1 /DEF = Homo sapiens | 205073_at |
| P450, subfamily | cytochrome P450, subfamily IIJ (arachidonic acid | |
| IIJ (arachidonic | epoxygenase) polypeptide 2 (CYP2J2), mRNA. | |
| acid | /FEA = mRNA /GEN = CYP2J2 | |
| epoxygenase) | /PROD = cytochrome P450, subfamily IIJ | |
| polypeptide 2 | (arachidonic acidepoxygenase) polypeptide 2 | |
| | /DB_XREF = gi: 4503226 /UG = Hs.152096 | |
| | cytochrome P450, subfamily IIJ (arachidonic acid | |
| | epoxygenase) polypeptide 2 /FL = gb: U37143.1 | |
| | gb: NM_000775.1 | |
| cystatin S | gb: NM_001899.1 /DEF = Homo sapiens cystatin S | 206994_at |
| | (CST4), mRNA. /FEA = mRNA /GEN = CST4 | |
| | /PROD = cystatin S /DB_XREF = gi: 4503108 | |
| | /UG = Hs.56319 cystatin S /FL = gb: NM_001899.1 | |
| spondin 1, (f- | Consensus includes gb: AI885290 /FEA = EST | 213993_at |
| spondin) | /DB_XREF = gi: 5590454 | |
| extracellular | /DB_XREF = est: wl92a04.x1 | |
| matrix protein | /CLONE = IMAGE: 2432334 /UG = Hs.5378 | |
| | spondin 1, (f-spondin) extracellular matrix | |
| | protein | |
| fibroblast growth | gb: NM_022969.1 /DEF = Homo sapiens fibroblast | 203638_s_at |
| factor receptor 2 | growth factor receptor 2 (bacteria-expressed | |
| (bacteria- | kinase, keratinocyte growth factor receptor, | |
| expressed kinase, | craniofacial dysostosis 1, Crouzon syndrome, | |
| keratinocyte | Pfeiffer syndrome, Jackson-Weiss syndrome) | |
| growth factor | (FGFR2), transcript variant 2, mRNA. | |
| receptor, | /FEA = mRNA /GEN = FGFR2 /PROD = fibroblast | |
| craniofacial | growth factor receptor 2, isoform 2precursor | |
| dysostosis 1, | /DB_XREF = gi: 13186252 /UG = Hs.278581 | |
| Crouzon | fibroblast growth factor receptor 2 (bacteria- | |
| syndrome, | expressed kinase, keratinocyte growth factor | |
| Pfeiffer | receptor, craniofacial dysostosis 1, Crouzon | |
| syndrome, | syndrome, Pfeiffer syndrome, Jackson-Weiss | |
| Jackson-Weiss | syndrome) /FL = gb: NM_022969.1 gb: M97193.1 | |
| syndrome) | gb: M80634.1 | |
| mucin 3B | Consensus includes gb: AB038783.1 /DEF = Homo | 214898_x_at |
| | sapiens MUC3B mRNA for intestinal mucin, | |
| | partial cds. /FEA = mRNA /GEN = MUC3B | |
| | /PROD = intestinal mucin /DB_XREF = gi: 9929917 | |
| | /UG = Hs.129782 mucin 3A, intestinal | |
| AA | Consensus includes gb: AV728958 /FEA = EST | 212703_at |
| | /DB_XREF = gi: 10838379 | |
| | /DB_XREF = est: AV728958 | |
| | /CLONE = HTCBYF04 /UG = Hs.150443 | |
| | KIAA0320 protein | |
| CUG triplet | gb: NM_006561.1 /DEF = Homo sapiens CUG | 202158_s_at |
| repeat, RNA- | triplet repeat, RNA-binding protein 2 (CUGBP2), | |
| binding protein 2 | mRNA. /FEA = mRNA /GEN = CUGBP2 | |
| | /PROD = CUG triplet repeat, RNA-binding protein | |
| | 2 /DB_XREF = gi: 5729815 /UG = Hs.211610 CUG | |
| | triplet repeat, RNA-binding protein 2 | |
| | /FL = gb: U69546.1 gb: AF036956.1 | |
| | gb: AF090694.1 gb: NM_006561.1 | |
| spondin 1, (f- | gb: AB051390.1 /DEF = Homo sapiens mRNA for | 209437_s_at |
| spondin) | VSGPF-spondin, complete cds. /FEA = mRNA | |
| extracellular | /PROD = VSGPF-spondin | |
| matrix protein | /DB_XREF = gi: 11320819 /UG = Hs.5378 spondin | |
| | 1, (f-spondin) extracellular matrix protein | |
| | /FL = gb: AB051390.1 | |
| mucin 3B | Consensus includes gb: AF113616 /DEF = Homo | 214676_x_at |
| | sapiens intestinal mucin 3 (MUC3) gene, partial | |
| | cds /FEA: = mRNA /DB_XREF = gi: 6466800 | |
| | /UG = Hs.129782 mucin 3A, intestinal | |
| EphA1 | gb: NM_005232.1 /DEF = Homo sapiens EphA1 | 205977_s_at |
| | (EPHA1), mRNA. /FEA = mRNA /GEN = EPHA1 | |
| | /PROD = EphA1 /DB_XREF = gi: 4885208 | |
| | /UG = Hs.89839 EphA1 /FL = gb: M18391.1 | |
| | gb: NM_005232.1 | |
| matrilin 3 | gb: NM_002381.2 /DEF = Homo sapiens matrilin 3 | 206091_at |
| | (MATN3) precursor, mRNA. /FEA = mRNA | |
| | /GEN = MATN3 /PROD = matrilin 3 precursor | |
| | /DB_XREF = gi: 13518040 /UG = Hs.278461 | |
| | matrilin 3 /FL = gb: NM_002381.2 | |
| bone | gb: NM_001200.1 /DEF = Homo sapiens bone | 205290_s_at |
| morphogenetic | morphogenetic protein 2 (BMP2), mRNA. | |
| protein 2 | /FEA = mRNA /GEN = BMP2 /PROD = bone | |
| | morphogenetic protein 2 precursor | |
| | /DB_XREF = gi: 4557368 /UG = Hs.73853 bone | |
| | morphogenetic protein 2 /FL = gb: NM_001200.1 | |
| interferon | Consensus includes gb: AI073984 /FEA = EST | 204057_at |
| consensus | /DB_XREF = gi: 3400628 | |
| sequence binding | /DB_XREF = est: oy66c05.x1 | |
| protein 1 | /CLONE = IMAGE: 1670792 /UG = Hs.14453 | |
| | interferon consensus sequence binding protein 1 | |
| | /FL = gb: M91196.1 gb: NM_002163.1 | |
| retinoic acid | Consensus includes gb: AI669229 /FEA = EST | 221872_at |
| receptor | /DB_XREF = gi: 4834003 | |
| responder | /DB_XREF = est: wc13e06.x1 | |
| (tazarotene | /CLONE = IMAGE: 2315074 /UG = Hs.82547 | |
| induced) 1 | retinoic acid receptor responder (tazarotene | |
| | induced) 1 | |
| cystic fibrosis | Consensus includes gb: W60595 /FEA = EST | 215703_at |
| transmembrane | /DB_XREF = gi: 1367354 | |
| conductance | /DB_XREF = est: zc91b04.s1 | |
| regulator, ATP- | /CLONE = IMAGE: 338479 /UG = Hs.663 cystic | |
| binding cassette | fibrosis transmembrane conductance regulator, | |
| (sub-family C, | ATP-binding cassette (sub-family C, member 7) | |
| member 7) | | |
| fibroblast growth | gb: M87771.1 /DEF = Human secreted fibroblast | 208228_s_at |
| factor receptor 2 | growth factor receptor (K-sam-III) mRNA, | |
| (bacteria- | complete cds. /FEA = mRNA /GEN = K-sam-III | |
| expressed kinase, | /PROD = fibroblast growth factor receptor | |
| keratinocyte | /DB_XREF = gi: 186781 /UG = Hs.278581 | |
| growth factor | fibroblast growth factor receptor 2 (bacteria- | |
| receptor, | expressed kinase, keratinocyte growth factor | |
| craniofacial | receptor, craniofacial dysostosis 1, Crouzon | |
| dysostosis 1, | syndrome, Pfeiffer syndrome, Jackson-Weiss | |
| Crouzon | syndrome) /FL = gb: NM_022970.1 gb: M87771.1 | |
| syndrome, | | |
| Pfeiffer | | |
| syndrome, | | |
| Jackson-Weiss | | |
| syndrome) | | |
| myosin, heavy | gb: NM_003802.1 /DEF = Homo sapiens myosin, | 208208_at |
| polypeptide 13, | heavy polypeptide 13, skeletal muscle (MYH13), | |
| skeletal muscle | mRNA. /FEA = mRNA /GEN = MYH13 | |
| | /PROD = myosin, heavy polypeptide 13, skeletal | |
| | muscle /DB_XREF = gi: 11321578 | |
| | /UG = Hs.278488 myosin, heavy polypeptide 13, | |
| | skeletal muscle /FL = gb: NM_003802.1 | |
| | gb: AF111782.2 | |
| ESTs, Weakly | Consensus includes gb: AW675655 /FEA = EST | 222354_at |
| similar to I38022 | /DB_XREF = gi: 7540890 | |
| hypothetical | /DB_XREF = est: ba52e01.x1 | |
| protein | /CLONE = IMAGE: 2900184 /UG = Hs.314158 | |
| [H. sapiens] | ESTs | |
| hypothetical | gb: NM_017699.1 /DEF = Homo sapiens | 219734_at |
| protein | hypothetical protein FLJ20174 (FLJ20174), | |
| FLJ20174 | mRNA. /FEA = mRNA /GEN = FLJ20174 | |
| | /PROD = hypothetical protein FLJ20174 | |
| | /DB_XREF = gi: 8923170 /UG = Hs.114556 | |
| | hypothetical protein FLJ20174 | |
| | /FL = gb: NM_017699.1 | |
| PTPRF | Consensus includes gb: AI692180 /FEA = EST | 212841_s_at |
| interacting | /DB_XREF = gi: 4969520 | |
| protein, binding | /DB_XREF = est: wd37f06.x1 | |
| protein 2 (liprin | /CLONE = IMAGE: 2330339 /UG = Hs.12953 | |
| beta 2) | PTPRF interacting protein, binding protein 2 | |
| | (liprin beta 2) | |
| ribonuclease, | gb: NM_002933.1 /DEF = Homo sapiens | 201785_at |
| RNase A family, | ribonuclease, RNase A family, 1 (pancreatic) | |
| 1 (pancreatic) | (RNASE1), mRNA. /FEA = mRNA | |
| | /GEN = RNASE1 /PROD = ribonuclease, RNase A | |
| | family, 1 (pancreatic) /DB_XREF = gi: 4506546 | |
| | /UG = Hs.78224 ribonuclease, RNase A family, 1 | |
| | (pancreatic) /FL = gb: BC005324.1 | |
| | gb: NM_002933.1 gb: D26129.1 | |
| hairless (mouse) | gb: NM_018411.1 /DEF = Homo sapiens hairless | 220163_s_at |
| homolog | protein (putative single zinc finger transcription | |
| | factor protein, responsible for autosomal | |
| | recessive universal congenital alopecia, HR gene) | |
| | (HSA277165), mRNA. /FEA = mRNA | |
| | /GEN = HSA277165 /PROD = hairless protein | |
| | /DB_XREF = gi: 11036651 /UG = Hs.272367 | |
| | hairless protein (putative single zinc finger | |
| | transcription factor protein, responsible for | |
| | autosomal recessive universal congenital | |
| | alopecia, HR gene) /FL = gb: NM_018411.1 | |
| nuclear receptor | Consensus includes gb: AF228413.1 /DEF = Homo | 210174_at |
| subfamily 5, | sapiens hepatocyte transcription factor mRNA, | |
| group A, | 3UTR. /FEA = mRNA /DB_XREF = gi: 7677372 | |
| member 2 | /UG = Hs.183123 nuclear receptor subfamily 5, | |
| | group A, member 2 /FL = gb: U93553.1 | |
| | gb: AB019246.1 gb: AF124247.1 | |
| superoxide | gb: NM_003102.1 /DEF = Homo sapiens | 205236_x_at |
| dismutase 3, | superoxide dismutase 3, extracellular (SOD3), | |
| extracellular | mRNA. /FEA = mRNA /GEN = SOD3 | |
| | /PROD = superoxide dismutase 3, extracellular | |
| | /DB_XREF = gi: 4507150 /UG = Hs.2420 | |
| | superoxide dismutase 3, extracellular | |
| | /FL = gb: J02947.1 gb: NM_003102.1 | |
| zinc finger | gb: NM_003438.1 /DEF = Homo sapiens zinc | 207394_at |
| protein 137 | finger protein 137 (clone pHZ-30) (ZNF137), | |
| (clone pHZ-30) | mRNA. /FEA = mRNA /GEN = ZNF137 | |
| | /PROD = zinc finger protein 137 (clone pHZ-30) | |
| | /DB_XREF = gi: 4507988 /UG = Hs.151689 zinc | |
| | finger protein 137 (clone pHZ-30) | |
| | /FL = gb: NM_003438.1 gb: U09414.1 | |
| Homo sapiens | Consensus includes gb: AL049983.1 /DEF = Homo | 217288_at |
| mRNA; cDNA | sapiens mRNA; cDNA DKFZp564D042 (from | |
| DKFZp564D042 | clone DKFZp564D042). /FEA = mRNA | |
| (from clone | /DB_XREF = gi: 4884234 /UG = Hs.240136 Homo | |
| DKFZp564D042) | sapiens mRNA; cDNA DKFZp564D042 (from | |
| | clone DKFZp564D042) | |
| Hermansky- | Consensus includes gb: AL022313 /DEF = Human | 217354_s_at |
| Pudlak syndrome | DNA sequence from clone RP5-1119A7 on | |
| | chromosome 22q12.2-12.3 Contains the TXN2 | |
| | gene for mitochondrial thioredoxin, a novel gene, | |
| | the EIF3S7 gene for eukaryotic translation | |
| | initiation factor 3 subunit 7 (zeta, 6667 kD) (EIF3- | |
| | P66), the gene f . . . /FEA = CDS_3 | |
| | /DB_XREF = gi: 4200326 /UG = Hs.272270 Human | |
| | DNA sequence from clone RP5-1119A7 on | |
| | chromosome 22q12.2-12.3 Contains the TXN2 | |
| | gene for mitochondrial thioredoxin, a novel gene, | |
| | the EIF3S7 gene for eukaryotic translation | |
| | initiation factor 3 subunit 7 (zeta, 6667 kD) (EIF3- | |
| | P66), the gene for a nov | |
| peroxisomal | gb: NM_018441.1 /DEF = Homo sapiens | 221142_s_at |
| trans 2-enoyl | peroxisomal trans 2-enoyl CoA reductase; | |
| CoA reductase; | putative short chain alcohol dehydrogenase | |
| putative short | (HSA250303), mRNA. /FEA = mRNA | |
| chain alcohol | /GEN = HSA250303 /PROD = peroxisomal trans 2- | |
| dehydrogenase | enoyl CoA reductase; putative short chain alcohol | |
| | dehydrogenase /DB_XREF = gi: 8923751 | |
| | /UG = Hs.281680 peroxisomal trans 2-enoyl CoA | |
| | reductase; putative short chain alcohol | |
| | dehydrogenase /FL = gb: NM_018441.1 | |
| BTG family, | gb: NM_006763.1 /DEF = Homo sapiens BTG | 201236_s_at |
| member 2 | family, member 2 (BTG2), mRNA. /FEA = mRNA | |
| | /GEN = BTG2 /PROD = BTG family, member 2 | |
| | /DB_XREF = gi: 5802987 /UG = Hs.75462 BTG | |
| | family, member 2 /FL = gb: U72649.1 | |
| | gb: NM_006763.1 | |
| phosducin | gb: M33478.1 /DEF = Human 33-kDa | 211496_s_at |
| | phototransducing protein mRNA, complete cds. | |
| | /FEA = mRNA /DB_XREF = gi: 177186 | |
| | /UG = Hs.550 phosducin /FL = gb: NM_022577.1 | |
| | gb: M33478.1 gb: AF076465.1 | |
| Rho GTPase | gb: NM_015366.1 /DEF = Homo sapiens Rho | 205980_s_at |
| activating protein 8 | GTPase activating protein 8 (ARHGAP8), | |
| | mRNA. /FEA = mRNA /GEN = ARHGAP8 | |
| | /PROD = Rho GTPase activating protein 8 | |
| | /DB_XREF = gi: 7656903 /UG = Hs.102336 Rho | |
| | GTPase activating protein 8 | |
| | /FL = gb: NM_015366.1 | |
| Homo sapiens | Consensus includes gb: AW593996 /FEA = EST | 213256_at |
| clone 24707 | /DB_XREF = gi: 7281254 | |
| mRNA sequence | /DB_XREF = est: hg41g06.x1 | |
| | /CLONE = IMAGE: 2948218 /UG = Hs.124969 | |
| | Homo sapiens clone 24707 mRNA sequence | |
| caspase 10, | gb: NM_001230.1 /DEF = Homo sapiens caspase | 205467_at |
| apoptosis-related | 10, apoptosis-related cysteine protease | |
| cysteine protease | (CASP10), mRNA. /FEA = mRNA | |
| | /GEN = CASP10 /PROD = caspase 10, apoptosis- | |
| | related cysteine protease /DB_XREF = gi: 4502568 | |
| | /UG = Hs.5353 caspase 10, apoptosis-related | |
| | cysteine protease /FL = gb: U60519.1 | |
| | gb: NM_001230.1 | |
| KIAA0690 | Consensus includes gb: AK000238.1 /DEF = Homo | 216360_x_at |
| protein | sapiens cDNA FLJ20231 fis, clone COLF5511, | |
| | highly similar to AB014590 Homo sapiens | |
| | mRNA for KIAA0690 protein. /FEA = mRNA | |
| | /DB_XREF = gi: 7020188 /UG = Hs.60103 | |
| | KIAA0690 protein | |
| Homo sapiens, | Consensus includes gb: AW001287 /FEA = EST | 227676_at |
| Similar to | /DB_XREF = gi: 5848203 | |
| RIKEN cDNA | /DB_XREF = est: wu27e06.x1 | |
| 1810037C20 | /CLONE = IMAGE: 2521282 /UG = Hs.61265 | |
| gene, clone | ESTs, Weakly similar to G786_HUMAN | |
| MGC: 21481 | PROTEIN GS3786 H. sapiens | |
| IMAGE: 385206 | | |
| 2, mRNA, | | |
| complete cds | | |
| ESTs | Consensus includes gb: AA581439 /FEA = EST | 244650_at |
| | /DB_XREF = gi: 2359211 | |
| | /DB_XREF = est: nh13c10.s1 | |
| | /CLONE = IMAGE: 952242 /UG = Hs.152328 | |
| | ESTs | |
| ESTs | Consensus includes gb: AI739241 /FEA = EST | 238984_at |
| | /DB_XREF = gi: 5101222 | |
| | /DB_XREF = est: wi14h02.x1 | |
| | /CLONE = IMAGE: 2390259 /UG = Hs.171480 | |
| | ESTs | |
| hypothetical | Consensus includes gb: AB046810.1 /DEF = Homo | 232083_at |
| protein | sapiens mRNA for KIAA1590 protein, partial | |
| FLJ23045 | cds. /FEA = mRNA /GEN = KIAA1590 | |
| | /PROD = KIAA1590 protein | |
| | /DB_XREF = gi: 10047254 /UG = Hs.101774 | |
| | hypothetical protein FLJ23045 | |
| regenerating | gb: AY007243.1 /DEF = Homo sapiens | 223447_at |
| gene type IV | regenerating gene type IV mRNA, complete cds. | |
| | /FEA = mRNA /PROD = regenerating gene type IV | |
| | /DB_XREF = gi: 12621025 /UG = Hs.105484 Homo | |
| | sapiens regenerating gene type IV mRNA, | |
| | complete cds /FL = gb: AY007243.1 | |
| ESTs | Consensus includes gb: AI139990 /FEA = EST | 231022_at |
| | /DB_XREF = gi: 3647447 | |
| | /DB_XREF = est: qa47d03.x1 | |
| | /CLONE = IMAGE: 1689893 /UG = Hs.134586 | |
| | ESTs | |
| ESTs | Consensus includes gb: AI733801 /FEA = EST | 237923_at |
| | /DB_XREF = gi: 5054914 | |
| | /DB_XREF = est: qk39c04.x5 | |
| | /CLONE = IMAGE: 1871334 /UG = Hs.146186 | |
| | ESTs | |
| hypothetical | Consensus includes gb: AK002203.1 /DEF = Homo | 226992_at |
| protein | sapiens cDNA FLJ11341 fis, clone | |
| MGC20702 | PLACE1010786. /FEA = mRNA | |
| | /DB_XREF = gi: 7023932 /UG = Hs.10260 Homo | |
| | sapiens cDNA FLJ11341 fis, clone | |
| | PLACE1010786 | |
| ESTs, Weakly | Consensus includes gb: AI457984 /FEA = EST | 243729_at |
| similar to | /DB_XREF = gi: 4312002 | |
| ALU1_HUMAN | /DB_XREF = est: tj66a04.x1 | |
| ALU | /CLONE = IMAGE: 2146446 /UG = Hs.165900 | |
| SUBFAMILY J | ESTs, Weakly similar to ALUC_HUMAN !!!! | |
| SEQUENCE | ALU CLASS C WARNING ENTRY !!! | |
| CONTAMINATION | H. sapiens | |
| WARNING | | |
| ENTRY | | |
| [H. sapiens] | | |
| Homo sapiens | Consensus includes gb: T86159 /FEA = EST | 227724_at |
| cDNA: | /DB_XREF = gi: 714511 | |
| FLJ22063 fis, | /DB_XREF = est: yd84h07.s1 | |
| clone HEP10326 | /CLONE = IMAGE: 114973 /UG = Hs.10450 | |
| | Homo sapiens cDNA: FLJ22063 fis, clone | |
| | HEP10326 | |
| ESTs | Consensus includes gb: AI806131 /FEA = EST | 231148_at |
| | /DB_XREF = gi: 5392697 | |
| | /DB_XREF = est: wf06c06.x1 | |
| | /CLONE = IMAGE: 2349802 /UG = Hs.99376 | |
| | ESTs | |
| anterior gradient | Consensus includes gb: AI922323 /FEA = EST | 228969_at |
| 2 (Xenepus | /DB_XREF = gi: 5658287 | |
| laevis) homolog | /DB_XREF = est: wn90h03.x1 | |
| | /CLONE = IMAGE: 2453141 /UG = Hs.293380 | |
| | ESTs | |
| ESTs | Consensus includes gb: AI493909 /FEA = EST | 235562_at |
| | /DB_XREF = gi: 4394912 | |
| | /DB_XREF = est: qz94e02.x1 | |
| | /CLONE = IMAGE: 2042234 /UG = Hs.6131 ESTs | |
| hypothetical | Consensus includes gb: AI339568 /FEA = EST | 222727_s_at |
| protein | /DB_XREF = gi: 4076495 | |
| FLJ22233 | /DB_XREF = est: qk67e10.x1 | |
| | /CLONE = IMAGE: 1874058 /UG = Hs.286194 | |
| | hypothetical protein FLJ22233 | |
| | /FL = gb: NM_024959.1 | |
| GalNAc alpha-2,6- | Consensus includes gb: Y11339.2 /DEF = Homo | 227725_at |
| sialyltransferase | sapiens mRNA for GalNAc alpha-2,6- | |
| I, long form | sialyltransferase I, long form. /FEA = mRNA | |
| | /PROD = GalNAc alpha-2,6-sialyltransferase I | |
| | /DB_XREF = gi: 7576275 /UG = Hs.105352 | |
| | GalNAc alpha-2,6-sialyltransferase I, long form | |
| ESTs | Consensus includes gb: AI917390 /FEA = EST | 240964_at |
| | /DB_XREF = gi: 5637245 | |
| | /DB_XREF = est: ts79a05.x1 | |
| | /CLONE = IMAGE: 2237456 /UG = Hs.99415 | |
| | ESTs | |
| Homo sapiens | Consensus includes gb: AK026404.1 /DEF = Homo | 232321_at |
| cDNA: | sapiens cDNA: FLJ22751 fis, clone KAIA0483, | |
| FLJ22751 fis, | highly similar to AF016692 Homo sapiens small | |
| clone | intestinal mucin (MUC3) mRNA. /FEA = mRNA | |
| KAIA0483, | /DB_XREF = gi: 10439257 /UG = Hs.271819 Homo | |
| highly similar to | sapiens cDNA: FLJ22751 fis, clone KAIA0483, | |
| AF016692 Homo | highly similar to AF016692 Homo sapiens small | |
| sapiens small | intestinal mucin (MUC3) mRNA | |
| intestinal mucin | | |
| (MUC3) mRNA | | |
| Homo sapiens | Consensus includes gb: AK026984.1 /DEF = Homo | 229021_at |
| cDNA: | sapiens cDNA: FLJ23331 fis, clone HEP12664. | |
| FLJ23331 fis, | /FEA = mRNA /DB_XREF = gi: 10439980 | |
| clone HEP12664 | /UG = Hs.50742 Homo sapiens cDNA: FLJ23331 | |
| | fis, clone HEP12664 | |
| ESTs | Consensus includes gb: AA827649 /FEA = EST | 235515_at |
| | /DB_XREF = gi: 2900090 | |
| | /DB_XREF = est: od01a12.s1 | |
| | /CLONE = IMAGE: 1357918 /UG = Hs.105317 | |
| | ESTs | |
| prostate cancer | Consensus includes gb: AA633076 /FEA = EST | 226167_at |
| associated | /DB_XREF = gi: 2556490 | |
| protein 7 | /DB_XREF = est: nq38a06.s1 | |
| | /CLONE = IMAGE: 1146130 /UG = Hs.27495 | |
| | prostate cancer associated protein 7 | |
| ESTs | Consensus includes gb: N37023 /FEA = EST | 225407_at |
| | /DB_XREF = gi: 1158165 | |
| | /DB_XREF = est: yy40d03.s1 | |
| | /CLONE = IMAGE: 273701 /UG = Hs.235883 | |
| | ESTs | |
| ESTs, Weakly | Consensus includes gb: AI864053 /FEA = EST | 235678_at |
| similar to I38588 | /DB_XREF = gi: 5528160 | |
| reverse | /DB_XREF = est: wj55h10.x1 | |
| transcriptase | /CLONE = IMAGE: 2406787 /UG = Hs.39972 | |
| homolog | ESTs, Weakly similar to I38588 reverse | |
| [H. sapiens] | transcriptase homolog H. sapiens | |
| ESTs, Weakly | Consensus includes gb: AA557324 /FEA = EST | 227702_at |
| similar to | /DB_XREF = gi: 2327801 | |
| JX0331 laurate | /DB_XREF = est: nl81a02.s1 | |
| omega- | /CLONE = IMAGE: 1057034 /UG = Hs.26040 | |
| hydroxylase | ESTs, Weakly similar to fatty acid omega- | |
| [H. sapiens] | hydroxylase H. sapiens | |
| ESTs | Consensus includes gb: BF594323 /FEA = EST | 238103_at |
| | /DB_XREF = gi: 11686647 | |
| | /DB_XREF = est: 7h79g07.x1 | |
| | /CLONE = IMAGE: 3322236 /UG = Hs.158989 | |
| | ESTs | |
| ESTs, Weakly | Consensus includes gb: AI827789 /FEA = EST | 228241_at |
| similar to | /DB_XREF = gi: 5448449 | |
| JE0350 Anterior | /DB_XREF = est: wf33a07.x1 | |
| gradient-2 | /CLONE = IMAGE: 2357364 /UG = Hs.100686 | |
| [H. sapiens] | ESTs, Weakly similar to JE0350 Anterior | |
| | gradient-2 H. sapiens | |
| ESTs | Consensus includes gb: AI968097 /FEA = EST | 237835_at |
| | /DB_XREF = gi: 5764915 | |
| | /DB_XREF = est: wu13a12.x1 | |
| | /CLONE = IMAGE: 2516830 /UG = Hs.131360 | |
| | ESTs | |
| ESTs | Consensus includes gb: H05025 /FEA = EST | 241874_at |
| | /DB_XREF = gi: 868577 | |
| | /DB_XREF = est: y174g12.s1 | |
| | /CLONE = IMAGE: 43864 /UG = Hs.323767 ESTs | |
| Homo sapiens, | Consensus includes gb: AA524690 /FEA = EST | 226168_at |
| Similar to | /DB_XREF = gi: 2265618 | |
| RIKEN cDNA | /DB_XREF = est: ng38e07.s1 | |
| 1110060O18 | /CLONE = IMAGE: 937092 /UG = Hs.294143 | |
| gene, clone | ESTs, Weakly similar to predicted using | |
| MGC: 17236 | Genefinder C. elegans | |
| IMAGE: 3864137, | | |
| mRNA, | | |
| complete cds | | |
| ESTs | Consensus includes gb: AI300126 /FEA = EST | 240830_at |
| | /DB_XREF = gi: 3959472 | |
| | /DB_XREF = est: qn54f02.x1 | |
| | /CLONE = IMAGE: 1902075 /UG = Hs.257858 | |
| | ESTs | |
| Homo sapiens | Consensus includes gb: AA129774 /FEA = EST | 227019_at |
| cDNA FLJ13137 | /DB_XREF = gi: 1690185 | |
| fis, clone | /DB_XREF = est: z116h09.s1 | |
| NT2RP3003150 | /CLONE = IMAGE: 502145 /UG = Hs.288905 | |
| | Homo sapiens cDNA FLJ13137 fis, clone | |
| | NT2RP3003150 | |
| ESTs | Consensus includes gb: AW024656 /FEA = EST | 242358_at |
| | /DB_XREF = gi: 5878186 | |
| | /DB_XREF = est: wu78h05.x1 | |
| | /CLONE = IMAGE: 2526201 /UG = Hs.233382 | |
| | ESTs, Moderately similar to AF119917 62 | |
| | PRO2822 H. sapiens | |

The biomarker probe set list B (Table 3) contains 95 probe sets (U133A: 47; U133B 48). The biomarker probe set list B contains polynucleotides identified to be biomarkers of EGFR antagonist sensitivity employing strategy B. In strategy B, polynucleotides were required to satisfy a stringent criteria for correlation to IC₅₀ values and a less stringent condition for EGFR status coregulation. Namely, the polynucleotides had to have a Pearsons correlation of −0.5 or less with respect to IC₅₀ and be called absent by the Affymetrix software in 5 out of the 6 cell lines with lowest expression of EGFR.

TABLE 3
Biomarker Probe Set List B

| | | Affymetrix |
|---|---|---|
| Unigene Title | Affymetrix Description | probe set |
| dopa | Consensus includes gb: AW772056 /FEA = EST | 214347_s_at |
| decarboxylase | /DB_XREF = gi: 7704118 | |
| (aromatic L- | /DB_XREF = est: hn64g06.x1 | |
| amino acid | /CLONE = IMAGE: 3032698 /UG = Hs.150403 | |
| decarboxylase) | dopa decarboxylase (aromatic L-amino acid | |
| | decarboxylase) | |
| cystic fibrosis | gb: NM_000492.2 /DEF = Homo sapiens cystic | 205043_at |
| transmembrane | fibrosis transmembrane conductance regulator, | |
| conductance | ATP-binding cassette (sub-family C, member 7) | |
| regulator, ATP- | (CFTR), mRNA. /FEA = mRNA /GEN = CFTR | |
| binding cassette | /PROD = cystic fibrosis transmembrane | |
| (sub-family C, | conductanceregulator, ATP-binding cassette | |
| member 7) | (sub-family C, member 7) | |
| | /DB_XREF = gi: 6995995 /UG = Hs.663 cystic | |
| | fibrosis transmembrane conductance regulator, | |
| | ATP-binding cassette (sub-family C, member 7) | |
| | /FL = gb: NM_000492.2 | |
| carcinoembryonic | gb: BC005008.1 /DEF = Homo sapiens, | 203757_s_at |
| antigen-related | carcinoembryonic antigen-related cell adhesion | |
| cell adhesion | molecule 6 (non-specific cross reacting antigen), | |
| molecule 6 (non- | clone MGC: 10467, mRNA, complete cds. | |
| specific cross | /FEA = mRNA /PROD = carcinoembryonic | |
| reacting antigen) | antigen-related cell adhesionmolecule 6 (non- | |
| | specific cross reacting antigen) | |
| | /DB_XREF = gi: 13477106 /UG = Hs.73848 | |
| | carcinoembryonic antigen-related cell adhesion | |
| | molecule 6 (non-specific cross reacting antigen) | |
| | /FL = gb: BC005008.1 gb: M18216.1 gb: M29541.1 | |
| | gb: NM_002483.1 | |
| hypothetical | gb: NM_017655.1 /DEF = Homo sapiens | 219970_at |
| protein | hypothetical protein FLJ20075 (FLJ20075), | |
| FLJ20075 | mRNA. /FEA = mRNA /GEN = FLJ20075 | |
| | /PROD = hypothetical protein FLJ20075 | |
| | /DB_XREF = gi: 8923083 /UG = Hs.205058 | |
| | hypothetical protein FLJ20075 | |
| | /FL = gb: NM_017655.1 | |
| ATPase, Class | Consensus includes gb: AW006935 /FEA = EST | 214070_s_at |
| V, type 10B | /DB_XREF = gi: 5855713 | |
| | /DB_XREF = est: wt08b11.x1 | |
| | /CLONE = IMAGE: 2506845 /UG = Hs.109358 | |
| | ATPase, Class V, type 10B | |
| cystic fibrosis | Consensus includes gb: W60595 /FEA = EST | 215702_s_at |
| transmembrane | /DB_XREF = gi: 1367354 | |
| conductance | /DB_XREF = est: zc91b04.s1 | |
| regulator, ATP- | /CLONE = IMAGE: 338479 /UG = Hs.663 cystic | |
| binding cassette | fibrosis transmembrane conductance regulator, | |
| (sub-family C, | ATP-binding cassette (sub-family C, member 7) | |
| member 7) | | |
| HERV-H LTR- | gb: NM_007072.1 /DEF = Homo sapiens HERV- | 220812_s_at |
| associating 2 | H LTR-associating 2 (HHLA2), mRNA. | |
| | /FEA = mRNA /GEN = HHLA2 /PROD = HERV-H | |
| | LTR-associating 2 /DB_XREF = gi: 5901963 | |
| | /UG = Hs.252351 HERV-H LTR-associating 2 | |
| | /FL = gb: AF126162.1 gb: NM_007072.1 | |
| AA | Consensus includes gb: AV728958 /FEA = EST | 212703_at |
| | /DB_XREF = gi: 10838379 | |
| | /DB_XREF = est: AV728958 | |
| | /CLONE = HTCBYF04 /UG = Hs.150443 | |
| | KIAA0320 protein | |
| hemoglobin, | Consensus includes gb: T50399 /FEA = EST | 214414_x_at |
| alpha 2 | /DB_XREF = gi: 652259 | |
| | /DB_XREF = est: yb30b11.s1 | |
| | /CLONE = IMAGE: 72669 /UG = Hs.251577 | |
| | hemoglobin, alpha 1 | |
| spondin 1, (f- | Consensus includes gb: AI885290 /FEA = EST | 213993_at |
| spondin) | /DB_XREF = gi: 5590454 | |
| extracellular | /DB_XREF = est: w192a04.x1 | |
| matrix protein | /CLONE = IMAGE: 2432334 /UG = Hs.5378 | |
| | spondin 1, (f-spondin) extracellular matrix | |
| | protein | |
| hemoglobin, | gb: BC005931.1 /DEF = Homo sapiens, | 211745_x_at |
| alpha 1 | hemoglobin, alpha 2, clone MGC: 14541, | |
| | mRNA, complete cds. /FEA = mRNA | |
| | /PROD = hemoglobin, alpha 2 | |
| | /DB_XREF = gi: 13543547 /FL = gb: BC005931.1 | |
| serine (or | gb: NM_002639.1 /DEF = Homo sapiens serine | 204855_at |
| cysteine) | (or cysteine) proteinase inhibitor, clade B | |
| proteinase | (ovalbumin), member 5 (SERPINB5), mRNA. | |
| inhibitor, clade B | /FEA = mRNA /GEN = SERPINB5 /PROD = serine | |
| (ovalbumin), | (or cysteine) proteinase inhibitor, cladeB | |
| member 5 | (ovalbumin), member 5 /DB_XREF = gi: 4505788 | |
| | /UG = Hs.55279 serine (or cysteine) proteinase | |
| | inhibitor, clade B (ovalbumin), member 5 | |
| | /FL = gb: NM_002639.1 gb: U04313.1 | |
| 3-hydroxy-3- | gb: NM_005518.1 /DEF = Homo sapiens 3- | 204607_at |
| methylglutaryl- | hydroxy-3-methylglutaryl-Coenyme A synthase | |
| Coenzyme A | 2 (mitochondrial) (HMGCS2), mRNA. | |
| synthase 2 | /FEA = mRNA /GEN = HMGCS2 /PROD = 3- | |
| (mitochondrial) | hydroxy-3-methylglutaryl-Coenyme A synthase | |
| | 2(mitochondrial) /DB_XREF = gi: 5031750 | |
| | /UG = Hs.59889 3-hydroxy-3-methylglutaryl- | |
| | Coenzyme A synthase 2 (mitochondrial) | |
| | /FL = gb: NM_005518.1 | |
| anterior gradient | gb: AF088867.1 /DEF = Homo sapiens putative | 209173_at |
| 2 (Xenepus | secreted protein XAG mRNA, complete cds. | |
| laevis) homolog | /FEA = mRNA /PROD = putative secreted protein | |
| | XAG /DB_XREF = gi: 6652811 /UG = Hs.91011 | |
| | anterior gradient 2 (Xenepus laevis) homolog | |
| | /FL = gb: AF007791.1 gb: AF038451.1 | |
| | gb: NM_006408.1 gb: AF088867.1 | |
| FXYD domain- | gb: BC005238.1 /DEF = Homo sapiens, FXYD | 202489_s_at |
| containing ion | domain-containing ion transport regulator 3, | |
| transport | clone MGC: 12265, mRNA, complete cds. | |
| regulator 3 | /FEA = mRNA /PROD = FXYD domain- | |
| | containing ion transport regulator3 | |
| | /DB_XREF = gi: 13528881 /UG = Hs.301350 | |
| | FXYD domain-containing ion transport regulator | |
| | 3 /FL = gb: NM_005971.2 gb: BC005238.1 | |
| dipeptidylpeptidase | gb: M80536.1 /DEF = H. sapiens dipeptidylpeptidase | 203716_s_at |
| IV (CD26, | IV (DPP4) mRNA, complete cds. | |
| adenosine | /FEA = mRNA /GEN = DPP4 /PROD = dipeptidylpeptidase | |
| deaminase | IV /DB_XREF = gi: 181569 | |
| complexing | /UG = Hs.44926 dipeptidylpeptidase IV (CD26, | |
| protein 2) | adenosine deaminase complexing protein 2) | |
| | /FL = gb: M80536.1 gb: NM_001935.1 | |
| cystic fibrosis | Consensus includes gb: W60595 /FEA = EST | 215703_at |
| transmembrane | /DB_XREF = gi: 1367354 | |
| conductance | /DB_XREF = est: zc91b04.s1 | |
| regulator, ATP- | /CLONE = IMAGE: 338479 /UG = Hs.663 cystic | |
| binding cassette | fibrosis transmembrane conductance regulator, | |
| (sub-family C, | ATP-binding cassette (sub-family C, member 7) | |
| member 7) | | |
| EphA1 | gb: NM_005232.1 /DEF = Homo sapiens EphA1 | 205977_s_at |
| | (EPHA1), mRNA. /FEA = mRNA /GEN = EPHA1 | |
| | /PROD = EphA1 /DB_XREF = gi: 4885208 | |
| | /UG = Hs.89839 EphA1 /FL = gb: M18391.1 | |
| | gb: NM_005232.1 | |
| spondin 1, (f- | Consensus includes gb: AI885290 /FEA = EST | 213994_s_at |
| spondin) | /DB_XREF = gi: 5590454 | |
| extracellular | /DB_XREF = est: w192a04.x1 | |
| matrix protein | /CLONE = IMAGE: 2432334 /UG = Hs.5378 | |
| | spondin 1, (f-spondin) extracellular matrix | |
| | protein | |
| CUG triplet | gb: NM_006561.1 /DEF = Homo sapiens CUG | 202158_s_at |
| repeat, RNA- | triplet repeat, RNA-binding protein 2 | |
| binding protein 2 | (CUGBP2), mRNA. /FEA = mRNA | |
| | /GEN = CUGBP2 /PROD = CUG triplet repeat, | |
| | RNA-binding protein 2 /DB_XREF = gi: 5729815 | |
| | /UG = Hs.211610 CUG triplet repeat, RNA- | |
| | binding protein 2 /FL = gb: U69546.1 | |
| | gb: AF036956.1 gb: AF090694.1 | |
| | gb: NM_006561.1 | |
| DKFZP434C091 | Consensus includes gb: AL080170.1 | 215047_at |
| protein | /DEF = Homo sapiens mRNA; cDNA | |
| | DKFZp434C091 (from clone DKFZp434C091); | |
| | partial cds. /FEA = mRNA | |
| | /GEN = DKFZp434C091 /PROD = hypothetical | |
| | protein /DB_XREF = gi: 5262639 /UG = Hs.51692 | |
| | DKFZP434C091 protein | |
| mucin 3B | Consensus includes gb: AF113616 /DEF = Homo | 214676_x_at |
| | sapiens intestinal mucin 3 (MUC3) gene, partial | |
| | cds /FEA = mRNA /DB_XREF = gi: 6466800 | |
| | /UG = Hs.129782 mucin 3A, intestinal | |
| potassium | gb: U90065.1 /DEF = Human potassium channel | 204678_s_at |
| channel, | KCNO1 mRNA, complete cds. /FEA = mRNA | |
| subfamily K, | /PROD = potassium channel KCNO1 | |
| member 1 | /DB_XREF = gi: 1916294 /UG = Hs.79351 | |
| (TWIK-1) | potassium channel, subfamily K, member 1 | |
| | (TWIK-1) /FL = gb: U33632.1 gb: U90065.1 | |
| | gb: U76996.1 gb: NM_002245.1 | |
| nuclear receptor | gb: NM_000901.1 /DEF = Homo sapiens nuclear | 205259_at |
| subfamily 3, | receptor subfamily 3, group C, member 2 | |
| group C, member 2 | (NR3C2), mRNA. /FEA = mRNA /GEN = NR3C2 | |
| | /PROD = nuclear receptor subfamily 3, group C, | |
| | member 2 /DB_XREF = gi: 4505198 | |
| | /UG = Hs.1790 nuclear receptor subfamily 3, | |
| | group C, member 2 /FL = gb: M16801.1 | |
| | gb: NM_000901.1 | |
| BTG family, | gb: NM_006763.1 /DEF = Homo sapiens BTG | 201236_s_at |
| member 2 | family, member 2 (BTG2), mRNA. | |
| | /FEA = mRNA /GEN = BTG2 /PROD = BTG | |
| | family, member 2 /DB_XREF = gi: 5802987 | |
| | /UG = Hs.75462 BTG family, member 2 | |
| | /FL = gb: U72649.1 gb: NM_006763.1 | |
| G protein- | gb: AF062006.1 /DEF = Homo sapiens orphan G | 210393_at |
| coupled receptor | protein-coupled receptor HG38 mRNA, | |
| 49 | complete cds. /FEA = mRNA /PROD = orphan G | |
| | protein-coupled receptor HG38 | |
| | /DB_XREF = gi: 3366801 /UG = Hs.285529 G | |
| | protein-coupled receptor 49 /FL = gb: AF062006.1 | |
| | gb: AF061444.1 gb: NM_003667.1 | |
| hypothetical | gb: NM_017640.1 /DEF = Homo sapiens | 219573_at |
| protein | hypothetical protein FLJ20048 (FLJ20048), | |
| FLJ20048 | mRNA. /FEA = mRNA /GEN = FLJ20048 | |
| | /PROD = hypothetical protein FLJ20048 | |
| | /DB_XREF = gi: 8923056 /UG = Hs.116470 | |
| | hypothetical protein FLJ20048 | |
| | /FL = gb: NM_017640.1 | |
| cytochrome | gb: NM_000775.1 /DEF = Homo sapiens | 205073_at |
| P450, subfamily | cytochrome P450, subfamily IIJ (arachidonic | |
| IIJ (arachidonic | acid epoxygenase) polypeptide 2 (CYP2J2), | |
| acid | mRNA. /FEA = mRNA /GEN = CYP2J2 | |
| epoxygenase) | /PROD = cytochrome P450, subfamily IIJ | |
| polypeptide 2 | (arachidonic acidepoxygenase) polypeptide 2 | |
| | /DB_XREF = gi: 4503226 /UG = Hs.152096 | |
| | cytochrome P450, subfamily IIJ (arachidonic | |
| | acid epoxygenase) polypeptide 2 | |
| | /FL = gb: U37143.1 gb: NM_000775.1 | |
| brain-specific | gb: NM_007030.1 /DEF = Homo sapiens brain- | 206179_s_at |
| protein p25 alpha | specific protein p25 alpha (p25), mRNA. | |
| | /FEA = mRNA /GEN = p25 /PROD = brain-specific | |
| | protein p25 alpha /DB_XREF = gi: 5902017 | |
| | /UG = Hs.29353 brain-specific protein p25 alpha | |
| | /FL = gb: AB017016.1 gb: NM_007030.1 | |
| mucin 2, | gb: NM_002457.1 /DEF = Homo sapiens mucin 2, | 204673_at |
| intestinal/trachea 1 | intestinaltracheal (MUC2), mRNA. | |
| | /FEA = mRNA /GEN = MUC2 /PROD = mucin 2, | |
| | intestinaltracheal /DB_XREF = gi: 4505284 | |
| | /UG = Hs.315 mucin 2, intestinaltracheal | |
| | /FL = gb: NM_002457.1 gb: L21998.1 | |
| hypothetical | gb: NM_017699.1 /DEF = Homo sapiens | 219734_at |
| protein | hypothetical protein FLJ20174 (FLJ20174), | |
| FLJ20174 | mRNA. /FEA = mRNA /GEN = FLJ20174 | |
| | /PROD = hypothetical protein FLJ20174 | |
| | /DB_XREF = gi: 8923170 /UG = Hs.114556 | |
| | hypothetical protein FLJ20174 | |
| | /FL = gb: NM_017699.1 | |
| metastasis- | gb: NM_004739.1 /DEF = Homo sapiens | 203444_s_at |
| associated 1-like 1 | metastasis-associated 1-like 1 (MTA1L1), | |
| | mRNA. /FEA = mRNA /GEN = MTA1L1 | |
| | /PROD = metastasis-associated 1-like 1 | |
| | /DB_XREF = gi: 4758739 /UG = Hs.173043 | |
| | metastasis-associated 1-like 1 | |
| | /FL = gb: AB016591.1 gb: NM_004739.1 | |
| | gb: AF295807.1 | |
| bone | gb: NM_001200.1 /DEF = Homo sapiens bone | 205290_s_at |
| morphogenetic | morphogenetic protein 2 (BMP2), mRNA. | |
| protein 2 | /FEA = mRNA /GEN = BMP2 /PROD = bone | |
| | morphogenetic protein 2 precursor | |
| | /DB_XREF = gi: 4557368 /UG = Hs.73853 bone | |
| | morphogenetic protein 2 /FL = gb: NM_001200.1 | |
| heparanase | gb: NM_006665.1 /DEF = Homo sapiens | 219403_s_at |
| | heparanase (HPSE), mRNA. /FEA = mRNA | |
| | /GEN = HPSE /PROD = heparanase | |
| | /DB_XREF = gi: 5729872 /UG = Hs.44227 | |
| | heparanase /FL = gb: AF165154.1 gb: AF152376.1 | |
| | gb: NM_006665.1 gb: AF084467.1 | |
| | gb: AF155510.1 | |
| tumor necrosis | gb: BC002794.1 /DEF = Homo sapiens, tumor | 209354_at |
| factor receptor | necrosis factor receptor superfamily, member 14 | |
| superfamily, | (herpesvirus entry mediator), clone MGC: 3753, | |
| member 14 | mRNA, complete cds. /FEA = mRNA | |
| (herpesvirus | /PROD = tumor necrosis factor receptor | |
| entry mediator) | superfamily, member 14 (herpesvirus entry | |
| | mediator) /DB_XREF = gi: 12803894 | |
| | /UG = Hs.279899 tumor necrosis factor receptor | |
| | superfamily, member 14 (herpesvirus entry | |
| | mediator) /FL = gb: BC002794.1 gb: U70321.1 | |
| | gb: U81232.1 gb: NM_003820.1 gb: AF153978.1 | |
| CUG triplet | Consensus includes gb: N36839 /FEA = EST | 202156_s_at |
| repeat, RNA- | /DB_XREF = gi: 1157981 | |
| binding protein 2 | /DB_XREF = est: yy35f07.s1 | |
| | /CLONE = IMAGE: 273253 /UG = Hs.211610 | |
| | CUG triplet repeat, RNA-binding protein 2 | |
| | /FL = gb: U69546.1 gb: AF036956.1 | |
| | gb: AF090694.1 gb: NM_006561.1 | |
| ESTs, | Consensus includes gb: R06655 /FEA = EST | 217546_at |
| Moderately | /DB_XREF = gi: 757275 | |
| similar to | /DB_XREF = est: yf10e02.r1 | |
| AF078844 1 | /CLONE = IMAGE: 126458 /UG = Hs.188518 | |
| hqp0376 protein | ESTs, Moderately similar to AF078844 1 | |
| [H. sapiens] | hqp0376 protein H. sapiens | |
| hairless (mouse) | gb: NM_018411.1 /DEF = Homo sapiens hairless | 220163_s_at |
| homolog | protein (putative single zinc finger transcription | |
| | factor protein, responsible for autosomal | |
| | recessive universal congenital alopecia, HR | |
| | gene) (HSA277165), mRNA. /FEA = mRNA | |
| | /GEN = HSA277165 /PROD = hairless protein | |
| | /DB_XREF = gi: 11036651 /UG = Hs.272367 | |
| | hairless protein (putative single zinc finger | |
| | transcription factor protein, responsible for | |
| | autosomal recessive universal congenital | |
| | alopecia, HR gene) /FL = gb: NM_018411.1 | |
| branched chain | Consensus includes gb: NM_005504.1 | 214452_at |
| aminotransferase | /DEF = Homo sapiens branched chain | |
| 1, cytosolic | aminotransferase 1, cytosolic (BCAT1), mRNA. | |
| | /FEA = CDS /GEN = BCAT1 /PROD = branched | |
| | chain aminotransferase 1, cytosolic | |
| | /DB_XREF = gi: 5031606 /UG = Hs.157205 | |
| | branched chain aminotransferase 1, cytosolic | |
| | /FL = gb: U21551.1 gb: NM_005504.1 | |
| pancreas- | gb: NM_016341.1 /DEF = Homo sapiens | 205112_at |
| enriched | pancreas-enriched phospholipase C | |
| phospholipase C | (LOC51196), mRNA. /FEA = mRNA | |
| | /GEN = LOC51196 /PROD = pancreas-enriched | |
| | phospholipase C /DB_XREF = gi: 7705940 | |
| | /UG = Hs.6733 pancreas-enriched phospholipase | |
| | C /FL = gb: AF190642.2 gb: AF117948.1 | |
| | gb: NM_016341.1 | |
| prostaglandin- | gb: NM_000963.1 /DEF = Homo sapiens | 204748_at |
| endoperoxide | prostaglandin-endoperoxide synthase 2 | |
| synthase 2 | (prostaglandin GH synthase and | |
| (prostaglandin | cyclooxygenase) (PTGS2), mRNA. | |
| G/H synthase | /FEA-mRNA /GEN = PTGS2 | |
| and | /PROD = prostaglandin-endoperoxide synthase | |
| cyclooxygenase) | 2(prostaglandin GH synthase and | |
| | cyclooxygenase) /DB_XREF = gi: 4506264 | |
| | /UG = Hs.196384 prostaglandin-endoperoxide | |
| | synthase 2 (prostaglandin GH synthase and | |
| | cyclooxygenase) /FL = gb: M90100.1 | |
| | gb: L15326.1 gb: NM_000963.1 | |
| phosphatase and | gb: NM_000314.1 /DEF = Homo sapiens | 204054_at |
| tensin homolog | phosphatase and tensin homolog (mutated in | |
| (mutated in | multiple advanced cancers 1) (PTEN), mRNA. | |
| multiple | /FEA = mRNA /GEN = PTEN | |
| advanced cancers | /PROD = phosphatase and tensin homolog | |
| 1) | (mutated inmultiple advanced cancers 1) | |
| | /DB_XREF = gi: 4506248 /UG = Hs.10712 | |
| | phosphatase and tensin homolog (mutated in | |
| | multiple advanced cancers 1) /FL = gb: U92436.1 | |
| | gb: U93051.1 gb: U96180.1 gb: NM_000314.1 | |
| retinoic acid | Consensus includes gb: AI669229 /FEA = EST | 221872_at |
| receptor | /DB_XREF = gi: 4834003 | |
| responder | /DB_XREF = est: wc13e06.x1 | |
| (tazarotene | /CLONE = IMAGE: 2315074 /UG = Hs.82547 | |
| induced) 1 | retinoic acid receptor responder (tazarotene | |
| | induced) 1 | |
| protease inhibitor | gb: NM_002638.1 /DEF = Homo sapiens protease | 203691_at |
| 3, skin-derived | inhibitor 3, skin-derived (SKALP) (PI3), mRNA. | |
| (SKALP) | /FEA = mRNA /GEN = PI3 /PROD = protease | |
| | inhibitor 3, skin-derived (SKALP) | |
| | /DB_XREF = gi: 4505786 /UG = Hs.112341 | |
| | protease inhibitor 3, skin-derived (SKALP) | |
| | /FL = gb: NM_002638.1 | |
| zinc finger | gb: NM_003438.1 /DEF = Homo sapiens zinc | 207394_at |
| protein 137 | finger protein 137 (clone pHZ-30) (ZNF137), | |
| (clone pHZ-30) | mRNA. /FEA = mRNA /GEN = ZNF137 | |
| | /PROD = zinc finger protein 137 (clone pHZ-30) | |
| | /DB_XREF = gi: 4507988 /UG = Hs.151689 zinc | |
| | finger protein 137 (clone pHZ-30) | |
| | /FL = gb: NM_003438.1 gb: U09414.1 | |
| myosin, light | gb: NM_002477.1 /DEF = Homo sapiens myosin, | 205145_s_at |
| polypeptide 5, | light polypeptide 5, regulatory (MYL5), mRNA. | |
| regulatory | /FEA = mRNA /GEN = MYL5 /PROD = myosin, | |
| | light polypeptide 5, regulatory | |
| | /DB_XREF = gi: 4505304 /UG = Hs.170482 | |
| | myosin, light polypeptide 5, regulatory | |
| | /FL = gb: L03785.1 gb: NM_002477.1 | |
| tumor necrosis | gb: NM_000043.1 /DEF = Homo sapiens tumor | 204781_s_at |
| factor receptor | necrosis factor receptor superfamily, member 6 | |
| superfamily, | (TNFRSF6), mRNA. /FEA = mRNA | |
| member 6 | /GEN = TNFRSF6 /PROD = apoptosis (APO-1) | |
| | antigen 1 /DB_XREF = gi: 4507582 | |
| | /UG = Hs.82359 tumor necrosis factor receptor | |
| | superfamily, member 6 /FL = gb: M67454.1 | |
| | gb: NM_000043.1 | |
| hypothetical | Consensus includes gb: AI339568 /FEA = EST | 222727_s_at |
| protein | /DB_XREF = gi: 4076495 | |
| FLJ22233 | /DB_XREF = est: qk67e10.x1 | |
| | /CLONE = IMAGE: 1874058 /UG = Hs.286194 | |
| | hypothetical protein FLJ22233 | |
| | /FL = gb: NM_024959.1 | |
| regenerating | gb: AY007243.1 /DEF = Homo sapiens | 223447_at |
| gene type IV | regenerating gene type IV mRNA, complete | |
| | cds. /FEA = mRNA /PROD = regenerating gene | |
| | type IV /DB_XREF = gi: 12621025 | |
| | /UG = Hs.105484 Homo sapiens regenerating | |
| | gene type IV mRNA, complete cds | |
| | /FL = gb: AY007243.1 | |
| Homo sapiens | Consensus includes gb: AK025615.1 | 225285_at |
| cDNA: | /DEF = Homo sapiens cDNA: FLJ21962 fis, | |
| FLJ21962 fis, | clone HEP05564. /FEA = mRNA | |
| clone HEP05564 | /DB_XREF = gi: 10438186 /UG = Hs.7567 Homo | |
| | sapiens cDNA: FLJ21962 fis, clone HEP05564 | |
| phosphoprotein | Consensus includes gb: AK000680.1 | 225626_at |
| associated with | /DEF = Homo sapiens cDNA FLJ20673 fis, | |
| glycosphingolipi | clone KAIA4464. /FEA = mRNA | |
| d-enriched | /DB_XREF = gi: 7020924 /UG = Hs.266175 | |
| microdomains | phosphoprotein associated with GEMs | |
| | /FL = gb: AF240634.1 gb: NM_018440.1 | |
| hypothetical | Consensus includes gb: BF111925 /FEA = EST | 226171_at |
| protein | /DB_XREF = gi: 10941704 | |
| FLJ20209 | /DB_XREF = est: 7138g05.x1 | |
| | /CLONE = IMAGE: 3523784 /UG = Hs.3685 | |
| | hypothetical protein FLJ20209 | |
| Homo sapiens | Consensus includes gb: AA532640 /FEA = EST | 226484_at |
| mRNA for | /DB_XREF = gi: 2276894 | |
| KIAA1190 | /DB_XREF = est: nj17c04.s1 | |
| protein, partial | /CLONE = IMAGE: 986598 /UG = Hs.206259 | |
| cds | Homo sapiens mRNA for KIAA1190 protein, | |
| | partial cds | |
| KIAA1543 | Consensus includes gb: AB040976.1 | 226494_at |
| protein | /DEF = Homo sapiens mRNA for KIAA1543 | |
| | protein, partial cds. /FEA = mRNA | |
| | /GEN = KIAA1543 /PROD = KIAA1543 protein | |
| | /DB_XREF = gi: 7959352 /UG = Hs.17686 | |
| | KIAA1543 protein | |
| hypothetical | Consensus includes gb: AW138767 /FEA = EST | 227180_at |
| protein | /DB_XREF = gi: 6143085 /DB_XREF = est: UI-H- | |
| FLJ23563 | BI1-aep-a-12-0-UI.s1 | |
| | /CLONE = IMAGE: 2719799 /UG = Hs.274256 | |
| | hypothetical protein FLJ23563 | |
| ESTs | Consensus includes gb: AW264333 /FEA = EST | 227320_at |
| | /DB_XREF = gi: 6641075 | |
| | /DB_XREF = est: xq98e01.x1 | |
| | /CLONE = IMAGE: 2758680 /UG = Hs.21835 | |
| | ESTs | |
| ESTs | Consensus includes gb: BF589359 /FEA = EST | 227354_at |
| | /DB_XREF = gi: 11681683 | |
| | /DB_XREF = est: nab25d01.x1 | |
| | /CLONE = IMAGE: 3266737 /UG = Hs.13256 | |
| | ESTs | |
| Homo sapiens, | Consensus includes gb: AW001287 /FEA = EST | 227676_at |
| Similar to | /DB_XREF = gi: 5848203 | |
| RIKEN cDNA | /DB_XREF = est: wu27e06.x1 | |
| 1810037C20 | /CLONE = IMAGE: 2521282 /UG = Hs.61265 | |
| gene, clone | ESTs, Weakly similar to G786_HUMAN | |
| MGC: 21481 | PROTEIN GS3786 H. sapiens | |
| IMAGE: 3852062, | | |
| mRNA, | | |
| complete cds | | |
| Homo sapiens | Consensus includes gb: T86159 /FEA = EST | 227724_at |
| cDNA: | /DB_XREF = gi: 714511 | |
| FLJ22063 fis, | /DB_XREF = est: yd84h07.s1 | |
| clone HEP10326 | /CLONE = IMAGE: 114973 /UG = Hs.10450 | |
| | Homo sapiens cDNA: FLJ22063 fis, clone | |
| | HEP10326 | |
| ESTs | Consensus includes gb: AI700341 /FEA = EST | 228653_at |
| | /DB_XREF = gi: 4988241 | |
| | /DB_XREF = est: wd06e10.x1 | |
| | /CLONE = IMAGE: 2327370 /UG = Hs.110406 | |
| | ESTs | |
| ESTs | Consensus includes gb: BG494007 /FEA = EST | 228716_at |
| | /DB_XREF = gi: 13455521 | |
| | /DB_XREF = est: 602542289F1 | |
| | /CLONE = IMAGE: 4673182 /UG = Hs.203213 | |
| | ESTs | |
| ESTs | Consensus includes gb: AI559300 /FEA = EST | 229331_at |
| | /DB_XREF = gi: 4509505 | |
| | /DB_XREF = est: tq43d03.x1 | |
| | /CLONE = IMAGE: 2211557 /UG = Hs.294140 | |
| | ESTs | |
| hypothetical | Consensus includes gb: AI830823 /FEA = EST | 229439_s_at |
| protein | /DB_XREF = gi: 5451416 | |
| | /DB_XREF = est: wj52b06.x1 | |
| | /CLONE = IMAGE: 2406419 /UG = Hs.95549 | |
| | hypothetical protein | |
| ESTs | Consensus includes gb: BF431989 /FEA = EST | 229657_at |
| | /DB_XREF = gi: 11444103 | |
| | /DB_XREF = est: nab84a05.x1 | |
| | /CLONE = IMAGE: 3274280 /UG = Hs.203213 | |
| | ESTs | |
| ESTs | Consensus includes gb: BF589413 /FEA = EST | 229893_at |
| | /DB_XREF = gi: 11681737 | |
| | /DB_XREF = est: nab26b11.x1 | |
| | /CLONE = IMAGE: 3267020 /UG = Hs.55501 | |
| | ESTs | |
| brain-specific | Consensus includes gb: BG055052 /FEA = EST | 230104_s_at |
| protein p25 alpha | /DB_XREF = gi: 12512386 | |
| | /DB_XREF = est: nac94g06.x1 | |
| | /CLONE = IMAGE: 3441995 /UG = Hs.29353 | |
| | brain-specific protein p25 alpha | |
| ESTs, Weakly | Consensus includes gb: BF110588 /FEA = EST | 230645_at |
| similar to | /DB_XREF = gi: 10940278 | |
| MMHUE4 | /DB_XREF = est: 7n39e12.x1 | |
| erythrocyte | /CLONE = IMAGE: 3567071 /UG = Hs.150478 | |
| membrane | ESTs, Weakly similar to KIAA0987 protein | |
| protein 4.1, | H. sapiens | |
| parent splice | | |
| form [H. sapiens] | | |
| ESTs | Consensus includes gb: BF592062 /FEA = EST | 230760_at |
| | /DB_XREF = gi: 11684386 | |
| | /DB_XREF = est: 7n98h06.x1 | |
| | /CLONE = IMAGE: 3572962 /UG = Hs.233890 | |
| | ESTs | |
| hepatocyte | Consensus includes gb: AI032108 /FEA = EST | 230914_at |
| nuclear factor 4, | /DB_XREF = gi: 3250320 | |
| alpha | /DB_XREF = est: ow92d11.x1 | |
| | /CLONE = IMAGE: 1654293 /UG = Hs.54424 | |
| | hepatocyte nuclear factor 4, alpha | |
| ESTs | Consensus includes gb: AW203959 /FEA = EST | 230944_at |
| | /DB_XREF = gi: 6503431 /DB_XREF = est: UI-H- | |
| | BIl-aeu-b-12-0-UI.s1 | |
| | /CLONE = IMAGE: 2720590 /UG = Hs.149532 | |
| | ESTs | |
| ESTs | Consensus includes gb: AI139990 /FEA = EST | 231022_at |
| | /DB_XREF = gi: 3647447 | |
| | /DB_XREF = est: qa47d03.x1 | |
| | /CLONE = IMAGE: 1689893 /UG = Hs.134586 | |
| | ESTs | |
| ESTs | Consensus includes gb: AI806131 /FEA = EST | 231148_at |
| | /DB_XREF = gi: 5392697 | |
| | /DB_XREF = est: wf06c06.x1 | |
| | /CLONE = IMAGE: 2349802 /UG = Hs.99376 | |
| | ESTs | |
| hypothetical | Consensus includes gb: AB046810.1 | 232083_at |
| protein | /DEF = Homo sapiens mRNA for KIAA1590 | |
| FLJ23045 | protein, partial cds. /FEA = mRNA | |
| | /GEN = KIAA1590 /PROD = KIAA1590 protein | |
| | /DB_XREF = gi: 10047254 /UG = Hs.101774 | |
| | hypothetical protein FLJ323045 | |
| Homo sapiens | Consensus includes gb: AC004908 /DEF = Homo | 232641_at |
| PAC clone RP5- | sapiens PAC clone RP5-855D21 /FEA = CDS_3 | |
| 855D21 | /DB_XREF = gi: 4156179 /UG = Hs.249181 | |
| | Homo sapiens PAC clone RP5-855D21 | |
| putative | Consensus includes gb: AJ251708.1 | 234669_x_at |
| microtubule- | /DEF = Homo sapiens partial mRNA for putative | |
| binding protein | microtubule-binding protein. /FEA = mRNA | |
| | /PROD = putative microtubule-binding protein | |
| | /DB_XREF = gi: 6491740 /UG = Hs.326544 | |
| | putative microtubule-binding protein | |
| ESTs | Consensus includes gb: AI741469 /FEA = EST | 234970_at |
| | /DB_XREF = gi: 5109757 | |
| | /DB_XREF = est: wg11b01.x1 | |
| | /CLONE = IMAGE: 2364745 /UG = Hs.57787 | |
| | ESTs | |
| ESTs | Consensus includes gb: AI417897 /FEA = EST | 235444_at |
| | /DB_XREF = gi: 4261401 | |
| | /DB_XREF = est: tg55b06.x1 | |
| | /CLONE = IMAGE: 2112659 /UG = Hs.235860 | |
| | ESTs | |
| ESTs | Consensus includes gb: AI493909 /FEA = EST | 235562_at |
| | /DB_XREF = gi: 4394912 | |
| | /DB_XREF = est: qz94e02.x1 | |
| | /CLONE = IMAGE: 2042234 /UG = Hs.6131 | |
| | ESTs | |
| ESTs | Consensus includes gb: AV741130 /FEA = EST | 235651_at |
| | /DB_XREF = gi: 10858711 | |
| | /DB_XREF = est: AV741130 | |
| | /CLONE = CBCATB06 /UG = Hs.173704 ESTs, | |
| | Moderately similar to ALU8_HUMAN ALU | |
| | SUBFAMILY SX SEQUENCE | |
| | CONTAMINATION WARNING ENTRY | |
| | H. sapiens | |
| ESTs | Consensus includes gb: AW339510 /FEA = EST | 235866_at |
| | /DB_XREF = gi: 6836136 | |
| | /DB_XREF = est: xz91h08.x1 | |
| | /CLONE = IMAGE: 2871615 /UG = Hs.42722 | |
| | ESTs | |
| ESTs | Consensus includes gb: AI076192 /FEA = EST | 236422_at |
| | /DB_XREF = gi: 3405370 | |
| | /DB_XREF = est: oz01g07.x1 | |
| | /CLONE = IMAGE: 1674108 /UG = Hs.131933 | |
| | ESTs | |
| ESTs | Consensus includes gb: AL044570 /FEA = EST | 236548_at |
| | /DB_XREF = gi: 5432785 | |
| | /DB_XREF = est: DKFZp434L082_s1 | |
| | /CLONE = DKFZp434L082 /UG = Hs.147975 | |
| | ESTs | |
| ESTs | Consensus includes gb: AI733801 /FEA = EST | 237923_at |
| | /DB_XREF = gi: 5054914 | |
| | /DB_XREF = est: qk39c04.x5 | |
| | /CLONE = IMAGE: 1871334 /UG = Hs.146186 | |
| | ESTs | |
| Homo sapiens, | Consensus includes gb: T69015 /FEA = EST | 238422_at |
| clone | /DB_XREF = gi: 680163 | |
| MGC: 16402 | /DB_XREF = est: yc31f04.s1 | |
| IMAGE: 39403606, | /CLONE = IMAGE: 82303 /UG = Hs.192728 | |
| mRNA, | ESTs | |
| complete cds | | |
| ESTs | Consensus includes gb: AA502384 /FEA = EST | 238956_at |
| | /DB_XREF = gi: 2237351 | |
| | /DB_XREF = est: ne27f11.s1 | |
| | /CLONE = IMAGE: 898605 /UG = Hs.151529 | |
| | ESTs | |
| ESTs | Consensus includes gb: AI739241 /FEA = EST | 238984_at |
| | /DB_XREF = gi: 5101222 | |
| | /DB_XREF = est: wi14h02.x1 | |
| | /CLONE = IMAGE: 2390259 /UG = Hs.171480 | |
| | ESTs | |
| ESTs | Consensus includes gb: AA088446 /FEA = EST | 239065_at |
| | /DB_XREF = gi: 1633958 | |
| | /DB_XREF = est: zl89f04.s1 | |
| | /CLONE = IMAGE: 511807 /UG = Hs.170298 | |
| | ESTs | |
| ESTs | Consensus includes gb: AI493046 /FEA = EST | 239148_at |
| | /DB_XREF = gi: 4394049 | |
| | /DB_XREF = est: qz49b04.x1 | |
| | /CLONE = IMAGE: 2030191 /UG = Hs.146133 | |
| | ESTs | |
| ESTs | Consensus includes gb: AI243098 /FEA = EST | 239966_at |
| | /DB_XREF = gi: 3838495 | |
| | /DB_XREF = est: qh26e03.x1 | |
| | /CLONE = IMAGE: 1845820 /UG = Hs.178398 | |
| | ESTs | |
| ESTs, Weakly | Consensus includes gb: AI633523 /FEA = EST | 240106_at |
| similar to | /DB_XREF = gi: 4684853 | |
| A49175 Motch B | /DB_XREF = est: th68b11.x1 | |
| protein - mouse | /CLONE = IMAGE: 2123805 /UG = Hs.44705 | |
| [M. musculus] | ESTs | |
| betacellulin | Consensus includes gb: AI620677 /FEA = EST | 241412_at |
| | /DB_XREF = gi: 4629803 | |
| | /DB_XREF = est: tu85e09.x1 | |
| | /CLONE = IMAGE: 2257864 /UG = Hs.154191 | |
| | ESTs | |
| ESTs | Consensus includes gb: BF696216 /FEA = EST | 242626_at |
| | /DB_XREF = gi: 11981624 | |
| | /DB_XREF = est: 602124536F1 | |
| | /CLONE = IMAGE: 4281632 /UG = Hs.188724 | |
| | ESTs | |
| ESTs | Consensus includes gb: N57929 /FEA = EST | 242978_x_at |
| | /DB_XREF = gi: 1201819 | |
| | /DB_XREF = est: yv61e06.s1 | |
| | /CLONE = IMAGE: 247234 /UG = Hs.48100 | |
| | ESTs | |
| ESTs, Weakly | Consensus includes gb: AI457984 /FEA = EST | 243729_at |
| similar to | /DB_XREF = gi: 4312002 | |
| ALU1_HUMAN | /DB_XREF = est: tj66a04.x1 | |
| ALU | /CLONE = IMAGE: 2146446 /UG = Hs.165900 | |
| SUBFAMILY J | ESTs, Weakly similar to ALUC_HUMAN !!!! | |
| SEQUENCE | ALU CLASS C WARNING ENTRY !!! | |
| CONTAMINATION | H. sapiens | |
| WARNING | | |
| ENTRY | | |
| [H. sapiens] | | |
| ESTs | Consensus includes gb: AA581439 /FEA = EST | 244650_at |
| | /DB_XREF = gi: 2359211 | |
| | /DB_XREF = est: nh13c10.s1 | |
| | /CLONE = IMAGE: 952242 /UG = Hs.152328 | |
| | ESTs | |

The two biomarker probe sets A and B were then combined, a total of 161 different probe sets, and the redundant polynucleotides were removed, representing 125 unique polynucleotides which are provided below in Table 4. The Table 4 polynucleotides are biomarkers of the invention.

TABLE 4
Biomarkers

| Unigene Title | | Affymetrix |
|---|---|---|
| And SEQ ID NO: | Affymetrix Description | probe set |
| 3-hydroxy-3- | gb: NM_005518.1 /DEF = Homo sapiens 3- | 204607_at |
| methylglutaryl- | hydroxy-3-methylglutaryl-Coenzyme A | |
| Coenzyme A | synthase 2 (mitochondrial) (HMGCS2), | |
| synthase 2 | mRNA. /FEA = mRNA /GEN = HMGCS2 | |
| (mitochondrial) | /PROD = 3-hydroxy-3-methylglutaryl- | |
| SEQ ID NOS: 1 | Coenzyme A synthase 2(mitochondrial) | |
| (DNA) and 126 | /DB_XREF = gi: 5031750 /UG = Hs.59889 3- | |
| (amino acid) | hydroxy-3-methylglutaryl-Coenzyme A | |
| | synthase 2 (mitochondrial) | |
| | /FL = gb: NM_005518.1 | |
| ATPase, Class V, | Consensus includes gb: AW006935 | 214070_s_at |
| type 10B | /FEA = EST /DB_XREF = gi: 5855713 | |
| | /DB_XREF = est: wt08b11.x1 | |
| SEQ ID NO: 2 | /CLONE = IMAGE: 2506845 /UG = Hs.109358 | |
| (DNA) | ATPase, Class V, type 10B | |
| bone morphogenetic | gb: NM_001200.1 /DEF = Homo sapiens bone | 205290_s_at |
| protein 2 | morphogenetic protein 2 (BMP2), mRNA. | |
| SEQ ID NOS: 3 | /FEA = mRNA /GEN = BMP2 /PROD = bone | |
| (DNA) and 127 | morphogenetic protein 2 precursor | |
| (amino acid) | /DB_XREF = gi: 4557368 /UG = Hs.73853 bone | |
| | morphogenetic protein 2 | |
| | /FL = gb: NM_001200.1 | |
| brain-specific protein | gb: NM_007030.1 /DEF = Homo sapiens brain- | 206179_s_at |
| p25 alpha | specific protein p25 alpha (p25), mRNA. | |
| SEQ ID NOS: 4 | /FEA = mRNA /GEN = p25 /PROD = brain- | |
| (DNA) and 128 | specific protein p25 alpha | |
| (amino acid) | /DB_XREF = gi: 5902017 /UG = Hs.29353 | |
| | brain-specific protein p25 alpha | |
| | /FL = gb: AB017016.1 gb: NM_007030.1 | |
| branched chain | Consensus includes gb: NM_005504.1 | 214452_at |
| aminotransferase 1, | /DEF = Homo sapiens branched chain | |
| cytosolic | aminotransferase 1, cytosolic (BCAT1), | |
| SEQ ID NOS: 5 | mRNA. /FEA = CDS /GEN = BCAT1 | |
| (DNA) and 129 | /PROD = branched chain aminotransferase 1, | |
| (amino acid) | cytosolic /DB_XREF = gi: 5031606 | |
| | /UG = Hs.157205 branched chain | |
| | aminotransferase 1, cytosolic | |
| | /FL = gb: U21551.1 gb: NM_005504.1 | |
| BTG family, member 2 | gb: NM_006763.1 /DEF = Homo sapiens BTG | 201236_s_at |
| SEQ ID NOS: 6 | family, member 2 (BTG2), mRNA. | |
| (DNA) and 130 | /FEA = mRNA /GEN = BTG2 /PROD = BTG | |
| (amino acid) | family, member 2 /DB_XREF = gi: 5802987 | |
| | /UG = Hs.75462 BTG family, member 2 | |
| | /FL = gb: U72649.1 gb: NM_006763.1 | |
| Carcinoembryonic | gb: BC005008.1 /DEF = Homo sapiens, | 203757_s_at |
| antigen-related cell | carcinoembryonic antigen-related cell | |
| adhesion molecule 6 | adhesion molecule 6 (non-specific cross | |
| (non-specific cross | reacting antigen), clone MGC: 10467, mRNA, | |
| reacting antigen) | complete cds. /FEA = mRNA | |
| SEQ ID NOS: 7 | /PROD = carcinoembryonic antigen-related | |
| (DNA) and 131 | cell adhesionmolecule 6 (non-specific cross | |
| (amino acid) | reacting antigen) /DB_XREF = gi: 13477106 | |
| | /UG = Hs.73848 carcinoembryonic antigen- | |
| | related cell adhesion molecule 6 (non-specific | |
| | cross reacting antigen) /FL = gb: BC005008.1 | |
| | gb: M18216.1 gb: M29541.1 gb: NM_002483.1 | |
| caspase 10, apoptosis- | gb: NM_001230.1 /DEF = Homo sapiens | 205467_at |
| related cysteine | caspase 10, apoptosis-related cysteine | |
| protease | protease (CASP10), mRNA. /FEA = mRNA | |
| SEQ ID NOS: 8 | /GEN = CASP10 /PROD = caspase 10, | |
| (DNA) and 132 | apoptosis-related cysteine protease | |
| (amino acid) | /DB_XREF = gi: 4502568 /UG = Hs.5353 | |
| | caspase 10, apoptosis-related cysteine | |
| | protease /FL = gb: U60519.1 gb: NM_001230.1 | |
| CUG triplet repeat, | gb: NM_006561.1 /DEF = Homo sapiens CUG | 202158_s_at |
| RNA-binding protein 2 | triplet repeat, RNA-binding protein 2 | |
| SEQ ID NOS: 9 | (CUGBP2), mRNA. /FEA = mRNA | |
| (DNA) and 133 | /GEN = CUGBP2 /PROD = CUG triplet repeat, | |
| (amino acid) | RNA-binding protein 2 | |
| | /DB_XREF = gi: 5729815 /UG = Hs.211610 | |
| | CUG triplet repeat, RNA-binding protein 2 | |
| | /FL = gb: U69546.1 gb: AF036956.1 | |
| | gb: AF090694.1 gb: NM_006561.1 | |
| cystatin S | gb: NM_001899.1 /DEF = Homo sapiens | 206994_at |
| SEQ ID NOS: 10 | cystatin S (CST4), mRNA. /FEA = mRNA | |
| (DNA) and 134 | /GEN = CST4 /PROD = cystatin S | |
| (amino acid) | /DB_XREF = gi: 4503108 /UG = Hs.56319 | |
| | cystatin S /FL = gb: NM_001899.1 | |
| cystic fibrosis | gb: NM_000492.2 /DEF = Homo sapiens cystic | 205043_at |
| transmembrane | fibrosis transmembrane conductance | |
| conductance | regulator, ATP-binding cassette (sub-family | |
| regulator, ATP- | C, member 7) (CFTR), mRNA. | |
| binding cassette (sub- | /FEA = mRNA /GEN = CFTR /PROD = cystic | |
| family C, member 7) | fibrosis transmembrane conductanceregulator, | |
| SEQ ID NOS: 11 | ATP-binding cassette (sub-family C, member | |
| (DNA) and 135 | 7) /DB_XREF = gi: 6995995 /UG = Hs.663 | |
| (amino acid) | cystic fibrosis transmembrane conductance | |
| | regulator, ATP-binding cassette (sub-family | |
| | C, member 7) /FL = gb: NM_000492.2 | |
| cytochrome P450, | gb: NM_000775.1 /DEF = Homo sapiens | 205073_at |
| subfamily IIJ | cytochrome P450, subfamily IIJ (arachidonic | |
| (arachidonic acid | acid epoxygenase) polypeptide 2 (CYP2J2), | |
| epoxygenase) | mRNA. /FEA = mRNA /GEN = CYP2J2 | |
| polypeptide 2 | /PROD = cytochrome P450, subfamily IIJ | |
| SEQ ID NOS: 12 | (arachidonic acidepoxygenase) polypeptide 2 | |
| (DNA) and 136 | /DB_XREF = gi: 4503226 /UG = Hs.152096 | |
| (amino acid) | cytochrome P450, subfamily IIJ (arachidonic | |
| | acid epoxygenase) polypeptide 2 | |
| | /FL = gb: U37143.1 gb: NM_000775.1 | |
| dipeptidylpeptidase | gb: M80536.1 /DEF = H. sapiens dipeptidylpeptidase | 203716_s_at |
| IV (CD26, adenosine | IV (DPP4) mRNA, complete cds. | |
| deaminase | /FEA = mRNA /GEN = DPP4 | |
| complexing protein 2) | /PROD = dipeptidyl peptidase IV | |
| SEQ ID NOS 13 | /DB_XREF = gi: 181569 /UG = Hs.44926 | |
| (DNA) and 137 | dipeptidylpeptidase IV (CD26, adenosine | |
| (amino acid) | deaminase complexing protein 2) | |
| | /FL = gb: M80536.1 gb: NM_001935.1 | |
| DKFZP434C091 | Consensus includes gb: AL080170.1 | 215047_at |
| protein | /DEF = Homo sapiens mRNA; cDNA | |
| SEQ ID NO: 14 | DKFZp434C091 (from clone | |
| (DNA) | DKFZp434C091); partial cds. /FEA = mRNA | |
| | /GEN = DKFZp434C091 /PROD = hypothetical | |
| | protein /DB_XREF = gi: 5262639 | |
| | /UG = Hs.51692 DKFZP434C091 protein | |
| dopa decarboxylase | Consensus includes gb: AW772056 | 214347_s_at |
| (aromatic L-amino | /FEA = EST /DB_XREF = gi: 7704118 | |
| acid decarboxylase) | /DB_XREF = est: hn64g06.x1 | |
| SEQ ID NO: 15 | /CLONE = IMAGE: 3032698 /UG = Hs.150403 | |
| (DNA) | dopa decarboxylase (aromatic L-amino acid | |
| | decarboxylase) | |
| EphA1 | gb: NM_005232.1 /DEF = Homo sapiens | 205977_s_at |
| SEQ ID NOS: 16 | EphA1 (EPHA1), mRNA. /FEA = mRNA | |
| (DNA) and 138 | /GEN = EPHA1 /PROD = EphA1 | |
| (amino acid) | /DB_XREF = gi: 4885208 /UG = Hs.89839 | |
| | EphA1 /FL = gb: M18391.1 gb: NM_005232.1 | |
| ESTs, Moderately | Consensus includes gb: R06655 /FEA = EST | 217546_at |
| similar to AF078844 | /DB_XREF = gi: 757275 | |
| 1 hqp0376 protein | /DB_XREF = est: yf10e02.r1 | |
| [H. sapiens] | /CLONE = IMAGE: 126458 /UG = Hs.188518 | |
| SEQ ID NO: 17 | ESTs, Moderately similar to AF078844 1 | |
| (DNA) | hqp0376 protein H. sapiens | |
| ESTs, Weakly similar | Consensus includes gb: AW675655 | 222354_at |
| to I38022 | /FEA = EST /DB_XREF = gi: 7540890 | |
| hypothetical protein | /DB_XREF = est: ba52e01.x1 | |
| [H. sapiens] | /CLONE = IMAGE: 2900184 /UG = Hs.314158 | |
| SEQ ID NO: 18 | ESTs | |
| (DNA) | | |
| fibroblast growth | gb: NM_022969.1 /DEF = Homo sapiens | 203638_s_at |
| factor receptor 2 | fibroblast growth factor receptor 2 (bacteria- | |
| (bacteria-expressed | expressed kinase, keratinocyte growth factor | |
| kinase, keratinocyte | receptor, craniofacial dysostosis 1, Crouzon | |
| growth factor | syndrome, Pfeiffer syndrome, Jackson-Weiss | |
| receptor, craniofacial | syndrome) (FGFR2), transcript variant 2, | |
| dysostosis 1, Crouzon | mRNA. /FEA = mRNA /GEN = FGFR2 | |
| syndrome, Pfeiffer | /PROD = fibroblast growth factor receptor 2, | |
| syndrome, Jackson- | isoform 2precursor /DB_XREF = gi: 13186252 | |
| Weiss syndrome) | /UG = Hs.278581 fibroblast growth factor | |
| SEQ ID NOS: 19 | receptor 2 (bacteria-expressed kinase, | |
| (DNA) and 139 | keratinocyte growth factor receptor, | |
| (amino acid) | craniofacial dysostosis 1, Crouzon syndrome, | |
| | Pfeiffer syndrome, Jackson-Weiss syndrome) | |
| | /FL = gb: NM_022969.1 gb: M97193.1 | |
| | gb: M80634.1 | |
| FXYD domain- | gb: BC005238.1 /DEF = Homo sapiens, FXYD | 202489_s_at |
| containing ion | domain-containing ion transport regulator 3, | |
| transport regulator 3 | clone MGC: 12265, mRNA, complete cds. | |
| SEQ ID NOS: 20 | /FEA = mRNA /PROD = FXYD domain- | |
| (DNA) and 140 | containing ion transport regulator3 | |
| (amino acid) | /DB_XREF = gi: 13528881 /UG = Hs.301350 | |
| | FXYD domain-containing ion transport | |
| | regulator 3 /FL = gb: NM_005971.2 | |
| | gb: BC005238.1 | |
| G protein-coupled | gb: AF062006.1 /DEF = Homo sapiens orphan | 210393_at |
| receptor 49 | G protein-coupled receptor HG38 mRNA, | |
| SEQ ID NOS: 21 | complete cds. /FEA = mRNA /PROD = orphan | |
| (DNA) and 141 | G protein-coupled receptor HG38 | |
| (amino acid) | /DB_XREF = gi: 3366801 /UG = Hs.285529 G | |
| | protein-coupled receptor 49 | |
| | /FL = gb: AF062006.1 gb: AF061444.1 | |
| | gb: NM_003667.1 | |
| hairless (mouse) | gb: NM_018411.1 /DEF = Homo sapiens | 220163_s_at |
| homolog | hairless protein (putative single zinc finger | |
| SEQ ID NOS: 22 | transcription factor protein, responsible for | |
| (DNA) and 142 | autosomal recessive universal congenital | |
| (amino acid) | alopecia, HR gene) (HSA277165), mRNA. | |
| | /FEA = mRNA /GEN = HSA277165 | |
| | /PROD = hairless protein | |
| | /DB_XREF = gi: 11036651 /UG = Hs.272367 | |
| | hairless protein (putative single zinc finger | |
| | transcription factor protein, responsible for | |
| | autosomal recessive universal congenital | |
| | alopecia, HR gene) /FL = gb: NM_018411.1 | |
| hemoglobin, alpha 1 | gb: BC005931.1 /DEF = Homo sapiens, | 211745_x_at |
| SEQ ID NOS: 23 | hemoglobin, alpha 2, clone MGC: 14541, | |
| (DNA) and 143 | mRNA, complete cds. /FEA = mRNA | |
| (amino acid) | /PROD = hemoglobin, alpha 2 | |
| | /DB_XREF = gi: 13543547 | |
| | /FL = gb: BC005931.1 | |
| hemoglobin, alpha 2 | Consensus includes gb: T50399 /FEA = EST | 214414_x_at |
| SEQ ID NO: 24 | /DB_XREF = gi: 652259 | |
| (DNA) | /DB_XREF = est: yb30b11.s1 | |
| | /CLONE = IMAGE: 72669 /UG = Hs.251577 | |
| | hemoglobin, alpha 1 | |
| heparanase | gb: NM_006665.1 /DEF = Homo sapiens | 219403_s_at |
| SEQ ID NOS: 25 | heparanase (HPSE), mRNA. /FEA = mRNA | |
| (DNA) and 144 | /GEN = HPSE /PROD = heparanase | |
| (amino acid) | /DB_XREF = gi: 5729872 /UG = Hs.44227 | |
| | heparanase /FL = gb: AF165154.1 | |
| | gb: AF152376.1 gb: NM_006665.1 | |
| | gb: AF084467.1 gb: AF155510.1 | |
| Hermansky-Pudlak | Consensus includes gb: AL022313 | 217354_s_at |
| syndrome | /DEF = Human DNA sequence from clone | |
| SEQ ID NOS: 26 | RP5-1119A7 on chromosome 22q12.2-12.3 | |
| (DNA) and 145 | Contains the TXN2 gene for mitochondrial | |
| (amino acid) | thioredoxin, a novel gene, the EIF3S7 gene | |
| | for eukaryotic translation initiation factor 3 | |
| | subunit 7 (zeta, 6667 kD) (EIF3-P66), the | |
| | gene f . . . /FEA = CDS_3 | |
| | /DB_XREF = gi: 4200326 /UG = Hs.272270 | |
| | Human DNA sequence from clone RP5- | |
| | 1119A7 on chromosome 22q12.2-12.3 | |
| | Contains the TXN2 gene for mitochondrial | |
| | thioredoxin, a novel gene, the EIF3S7 gene | |
| | for eukaryotic translation initiation factor 3 | |
| | subunit 7 (zeta, 6667 kD) (EIF3-P66), the | |
| | gene for a nov | |
| HERV-H LTR- | gb: NM_007072.1 /DEF = Homo sapiens | 220812_s_at |
| associating 2 | HERV-H LTR-associating 2 (HHLA2), | |
| SEQ ID NOS: 27 | mRNA. /FEA = mRNA /GEN = HHLA2 | |
| (DNA) and 146 | /PROD = HERV-H LTR-associating 2 | |
| (amino acid) | /DB_XREF = gi: 5901963 /UG = Hs.252351 | |
| | HERV-H LTR-associating 2 | |
| | /FL = gb: AF126162.1 gb: NM_007072.1 | |
| Homo sapiens clone | Consensus includes gb: AW593996 | 213256_at |
| 24707 mRNA | /FEA = EST /DB_XREF = gi: 7281254 | |
| sequence | /DB_XREF = est: hg41g06.x1 | |
| SEQ ID NO: 28 | /CLONE = IMAGE: 2948218 /UG = Hs.124969 | |
| (DNA) | Homo sapiens clone 24707 mRNA sequence | |
| Homo sapiens | Consensus includes gb: AL049983.1 | 217288_at |
| mRNA; cDNA | /DEF = Homo sapiens mRNA; cDNA | |
| DKFZp564D042 | DKFZp564D042 (from clone | |
| (from clone | DKFZp564D042). /FEA = mRNA | |
| DKFZp564D042) | /DB_XREF = gi: 4884234 /UG = Hs.240136 | |
| SEQ ID NO: 29 | Homo sapiens mRNA; cDNA | |
| (DNA) | DKFZp564D042 (from clone | |
| | DKFZp564D042) | |
| hypothetical protein | gb: NM_017640.1 /DEF = Homo sapiens | 219573_at |
| FLJ20048 | hypothetical protein FLJ20048 (FLJ20048), | |
| SEQ ID NOS: 30 | mRNA. /FEA = mRNA /GEN = FLJ20048 | |
| (DNA) and 147 | /PROD = hypothetical protein FLJ20048 | |
| (amino acid) | /DB_XREF = gi: 8923056 /UG = Hs.116470 | |
| | hypothetical protein FLJ20048 | |
| | /FL = gb: NM_017640.1 | |
| hypothetical protein | gb: NM_017655.1 /DEF = Homo sapiens | 219970_at |
| FLJ20075 | hypothetical protein FLJ20075 (FLJ20075), | |
| SEQ ID NOS: 31 | mRNA. /FEA = mRNA /GEN = FLJ20075 | |
| (DNA) and 148 | /PROD = hypothetical protein FLJ20075 | |
| (amino acid) | /DB_XREF = gi: 8923083 /UG = Hs.205058 | |
| | hypothetical protein FLJ20075 | |
| | /FL = gb: NM_017655.1 | |
| interferon consensus | Consensus includes gb: AI073984 /FEA = EST | 204057_at |
| sequence binding | /DB_XREF = gi: 3400628 | |
| protein 1 | /DB_XREF = est: oy66c05.x1 | |
| SEQ ID NO: 32 | /CLONE = IMAGE: 1670792 /UG = Hs.14453 | |
| (DNA) | interferon consensus sequence binding protein | |
| | 1 /FL = gb: M91196.1 gb: NM_002163.1 | |
| KIAA0690 protein | Consensus includes gb: AK000238.1 | 216360_x_at |
| SEQ ID NO: 33 | /DEF = Homo sapiens cDNA FLJ20231 fis, | |
| (DNA) | clone COLF5511, highly similar to | |
| | AB014590 Homo sapiens mRNA for | |
| | KIAA0690 protein. /FEA = mRNA | |
| | /DB_XREF = gi: 7020188 /UG = Hs.60103 | |
| | KIAA0690 protein | |
| matrilin 3 | gb: NM_002381.2 /DEF = Homo sapiens | 206091_at |
| SEQ ID NOS: 34 | matrilin 3 (MATN3) precursor, mRNA. | |
| (DNA) and 149 | /FEA = mRNA /GEN = MATN3 | |
| (amino acid) | /PROD = matrilin 3 precursor | |
| | /DB_XREF = gi: 13518040 /UG = Hs.278461 | |
| | matrilin 3 /FL = gb: NM_002381.2 | |
| metastasis-associated | gb: NM_004739.1 /DEF = Homo sapiens | 203444_s_at |
| 1-like 1 | metastasis-associated 1-like 1 (MTA1L1), | |
| SEQ ID NOS: 35 | mRNA. /FEA = mRNA /GEN = MTA1L1 | |
| (DNA) and 150 | /PROD = metastasis-associated 1-like 1 | |
| (amino acid) | /DB_XREF = gi: 4758739 /UG = Hs.173043 | |
| | metastasis-associated 1-like 1 | |
| | /FL = gb: AB016591.1 gb: NM_004739.1 | |
| | gb: AF295807.1 | |
| mucin 2, | gb: NM_002457.1 /DEF = Homo sapiens mucin | 204673_at |
| intestinal/tracheal | 2, intestinaltracheal (MUC2), mRNA. | |
| SEQ ID NOS: 36 | /FEA = mRNA /GEN = MUC2 /PROD = mucin | |
| (DNA) and 151 | 2, intestinaltracheal /DB_XREF = gi: 4505284 | |
| (amino acid) | /UG = Hs.315 mucin 2, intestinaltracheal | |
| | /FL = gb: NM_002457.1 gb: L21998.1 | |
| mucin 3B | Consensus includes gb: AB038783.1 | 214898_x_at |
| SEQ ID NOS: 37 | /DEF = Homo sapiens MUC3B mRNA for | |
| (DNA) and 152 | intestinal mucin, partial cds. /FEA = mRNA | |
| (amino acid) | /GEN = MUC3B /PROD = intestinal mucin | |
| | /DB_XREF = gi: 9929917 /UG = Hs.129782 | |
| | mucin 3A, intestinal | |
| myosin, heavy | gb: NM_003802.1 /DEF = Homo sapiens | 208208_at |
| polypeptide 13, | myosin, heavy polypeptide 13, skeletal | |
| skeletal muscle | muscle (MYH13), mRNA. /FEA = mRNA | |
| SEQ ID NOS: 38 | /GEN = MYH13 /PROD = myosin, heavy | |
| (DNA) and 153 | polypeptide 13, skeletal muscle | |
| (amino acid) | /DB_XREF = gi: 11321578 /UG = Hs.278488 | |
| | myosin, heavy polypeptide 13, skeletal | |
| | muscle /FL = gb: NM_003802.1 | |
| | gb: AF111782.2 | |
| myosin, light | gb: NM_002477.1 /DEF = Homo sapiens | 205145_s_at |
| polypeptide 5, | myosin, light polypeptide 5, regulatory | |
| regulatory | (MYL5), mRNA. /FEA = mRNA | |
| SEQ ID NOS: 39 | /GEN = MYL5 /PROD = myosin, light | |
| (DNA) and 154 | polypeptide 5, regulatory | |
| (amino acid) | /DB_XREF = gi: 4505304 /UG = Hs.170482 | |
| | myosin, light polypeptide 5, regulatory | |
| | /FL = gb: L03785.1 gb: NM_002477.1 | |
| nuclear receptor | gb: NM_000901.1 /DEF = Homo sapiens | 205259_at |
| subfamily 3, group C, | nuclear receptor subfamily 3, group C, | |
| member 2 | member 2 (NR3C2), mRNA. /FEA = mRNA | |
| SEQ ID NOS: 40 | /GEN = NR3C2 /PROD = nuclear receptor | |
| (DNA) and 155 | subfamily 3, group C, member 2 | |
| (amino acid) | /DB_XREF = gi: 4505198 /UG = Hs.1790 | |
| | nuclear receptor subfamily 3, group C, | |
| | member 2 /FL = gb: M16801.1 | |
| | gb: NM_000901.1 | |
| nuclear receptor | Consensus includes gb: AF228413.1 | 210174_at |
| subfamily 5, group A, | /DEF = Homo sapiens hepatocyte transcription | |
| member 2 | factor mRNA, 3UTR. /FEA = mRNA | |
| SEQ ID NOS: 41 | /DB_XREF = gi: 7677372 /UG = Hs.183123 | |
| (DNA) and 156 | nuclear receptor subfamily 5, group A, | |
| (amino acid) | member 2 /FL = gb: U93553.1 gb: AB019246.1 | |
| | gb: AF124247.1 | |
| pancreas-enriched | gb: NM_016341.1 /DEF = Homo sapiens | 205112_at |
| phospholipase C | pancreas-enriched phospholipase C | |
| SEQ ID NOS: 42 | (LOC51196), mRNA. /FEA = mRNA | |
| (DNA) and 157 | /GEN = LOC51196 /PROD = pancreas-enriched | |
| (amino acid) | phospholipase C /DB_XREF = gi: 7705940 | |
| | /UG = Hs.6733 pancreas-enriched | |
| | phospholipase C /FL = gb: AF190642.2 | |
| | gb: AF117948.1 gb: NM_016341.1 | |
| peroxisomal trans 2- | gb: NM_018441.1 /DEF = Homo sapiens | 221142_s_at |
| enoyl CoA reductase; | peroxisomal trans 2-enoyl CoA reductase; | |
| putative short chain | putative short chain alcohol dehydrogenase | |
| alcohol | (HSA250303), mRNA. /FEA = mRNA | |
| dehydrogenase | /GEN = HSA250303 /PROD = peroxisomal | |
| SEQ ID NOS: 43 | trans 2-enoyl CoA reductase; putative short | |
| (DNA) and 158 | chain alcohol dehydrogenase | |
| (amino acid) | /DB_XREF = gi: 8923751 /UG = Hs.281680 | |
| | peroxisomal trans 2-enoyl CoA reductase; | |
| | putative short chain alcohol dehydrogenase | |
| | /FL = gb: NM_018441.1 | |
| phosducin | gb: M33478.1 /DEF = Human 33-kDa | 211496_s_at |
| SEQ ID NOS: 44 | phototransducing protein mRNA, complete | |
| (DNA) and 159 | cds. /FEA = mRNA /DB_XREF = gi: 177186 | |
| (amino acid) | /UG = Hs.550 phosducin | |
| | /FL = gb: NM_022577.1 gb: M33478.1 | |
| | gb: AF076465.1 | |
| phosphatase and | gb: NM_000314.1 /DEF = Homo sapiens | 204054_at |
| tensin homolog | phosphatase and tensin homolog (mutated in | |
| (mutated in multiple | multiple advanced cancers 1) (PTEN), | |
| advanced cancers 1) | mRNA. /FEA = mRNA /GEN = PTEN | |
| SEQ ID NOS: 45 | /PROD = phosphatase and tensin homolog | |
| (DNA) and 160 | (mutated inmultiple advanced cancers 1) | |
| (amino acid) | /DB_XREF = gi: 4506248 /UG = Hs.10712 | |
| | phosphatase and tensin homolog (mutated in | |
| | multiple advanced cancers 1) | |
| | /FL = gb: U92436.1 gb: U93051.1 gb: U96180.1 | |
| | gb: NM_000314.1 | |
| potassium channel, | gb: U90065.1 /DEF = Human potassium | 204678_s_at |
| subfamily K, member | channel KCNO1 mRNA, complete cds. | |
| 1 (TWIK-1) | /FEA = mRNA /PROD = potassium channel | |
| SEQ ID NOS: 46 | KCNO1 /DB_XREF = gi: 1916294 | |
| (DNA) and 161 | /UG = Hs.79351 potassium channel, subfamily | |
| (amino acid) | K, member 1 (TWIK-1) /FL = gb: U33632.1 | |
| | gb: U90065.1 gb: U76996.1 gb: NM_002245.1 | |
| prostaglandin- | gb: NM_000963.1 /DEF = Homo sapiens | 204748_at |
| endoperoxide | prostaglandin-endoperoxide synthase 2 | |
| synthase 2 | (prostaglandin GH synthase and | |
| (prostaglandin G/H | cyclooxygenase) (PTGS2), mRNA. | |
| synthase and | /FEA = mRNA /GEN = PTGS2 | |
| cyclooxygenase) | /PROD = prostaglandin-endoperoxide synthase | |
| SEQ ID NOS: 47 | 2(prostaglandin GH synthase and | |
| (DNA) and 162 | cyclooxygenase) /DB_XREF = gi: 4506264 | |
| (amino acid) | /UG = Hs.196384 prostaglandin-endoperoxide | |
| | synthase 2 (prostaglandin GH synthase and | |
| | cyclooxygenase) /FL = gb: M90100.1 | |
| | gb: L15326.1 gb: NM_000963.1 | |
| protease inhibitor 3, | gb: NM_002638.1 /DEF = Homo sapiens | 203691_at |
| skin-derived | protease inhibitor 3, skin-derived (SKALP) | |
| (SKALP) | (PI3), mRNA. /FEA = mRNA /GEN = PI3 | |
| SEQ ID NOS: 48 | /PROD = protease inhibitor 3, skin-derived | |
| (DNA) and 163 | (SKALP) /DB_XREF = gi: 4505786 | |
| (amino acid) | /UG = Hs.112341 protease inhibitor 3, skin- | |
| | derived (SKALP) /FL = gb: NM_002638.1 | |
| PTPRF interacting | Consensus includes gb: AI692180 /FEA = EST | 212841_s_at |
| protein, binding | /DB_XREF = gi: 4969520 | |
| protein 2 (liprin beta | /DB_XREF = est: wd37f06.x1 | |
| 2) | /CLONE = IMAGE: 2330339 /UG = Hs.12953 | |
| SEQ ID NO: 49 | PTPRF interacting protein, binding protein 2 | |
| (DNA) | (liprin beta 2) | |
| retinoic acid receptor | Consensus includes gb: AI669229 /FEA = EST | 221872_at |
| responder (tazarotene | /DB_XREF = gi: 4834003 | |
| induced) 1 | /DB_XREF = est: wc13e06.x1 | |
| SEQ ID NO: 50 | /CLONE = IMAGE: 2315074 /UG = Hs.82547 | |
| (DNA) | retinoic acid receptor responder (tazarotene | |
| | induced) 1 | |
| Rho GTPase | gb: NM_015366.1 /DEF = Homo sapiens Rho | 205980_s_at |
| activating protein 8 | GTPase activating protein 8 (ARHGAP8), | |
| SEQ ID NOS: 51 | mRNA. /FEA = mRNA /GEN = ARHGAP8 | |
| (DNA) and 164 | /PROD = Rho GTPase activating protein 8 | |
| (amino acid) | /DB_XREF = gi: 7656903 /UG = Hs.102336 | |
| | Rho GTPase activating protein 8 | |
| | /FL = gb: NM_015366.1 | |
| ribonuclease, RNase | gb: NM_002933.1 /DEF = Homo sapiens | 201785_at |
| A family, 1 | ribonuclease, RNase A family, 1 (pancreatic) | |
| (pancreatic) | (RNASE1), mRNA. /FEA = mRNA | |
| SEQ ID NOS: 52 | /GEN = RNASE1 /PROD = ribonuclease, | |
| (DNA) and 165 | RNase A family, 1 (pancreatic) | |
| (amino acid) | /DB_XREF = gi: 4506546 /UG = Hs.78224 | |
| | ribonuclease, RNase A family, 1 (pancreatic) | |
| | /FL = gb: BC005324.1 gb: NM_002933.1 | |
| | gb: D26129.1 | |
| serine (or cysteine) | gb: NM_002639.1 /DEF = Homo sapiens serine | 204855_at |
| proteinase inhibitor, | (or cysteine) proteinase inhibitor, clade B | |
| clade B (ovalbumin), | (ovalbumin), member 5 (SERPINB5), | |
| member 5 | mRNA. /FEA = mRNA /GEN = SERPINB5 | |
| SEQ ID NOS: 53 | /PROD = serine (or cysteine) proteinase | |
| (DNA) and 166 | inhibitor, cladeB (ovalbumin), member 5 | |
| (amino acid) | /DB_XREF = gi: 4505788 /UG = Hs.55279 | |
| | serine (or cysteine) proteinase inhibitor, clade | |
| | B (ovalbumin), member 5 | |
| | /FL = gb: NM_002639.1 gb: U04313.1 | |
| spondin 1, (f-spondin) | Consensus includes gb: AI885290 /FEA = EST | 213994_s_at |
| extracellular matrix | /DB_XREF = gi: 5590454 | |
| protein | /DB_XREF = est: wl92a04.x1 | |
| SEQ ID NO: 54 | /CLONE = IMAGE: 2432334 /UG = Hs.5378 | |
| (DNA) | spondin 1, (f-spondin) extracellular matrix | |
| | protein | |
| superoxide dismutase | gb: NM_003102.1 /DEF = Homo sapiens | 205236_x_at |
| 3, extracellular | superoxide dismutase 3, extracellular (SOD3), | |
| SEQ ID NOS: 55 | mRNA. /FEA = mRNA /GEN = SOD3 | |
| (DNA) and 167 | /PROD = superoxide dismutase 3, extracellular | |
| (amino acid) | /DB_XREF = gi: 4507150 /UG = Hs.2420 | |
| | superoxide dismutase 3, extracellular | |
| | /FL = gb: J02947.1 gb: NM_003102.1 | |
| tumor necrosis factor | gb: BC002794.1 /DEF = Homo sapiens, tumor | 209354_at |
| receptor superfamily, | necrosis factor receptor superfamily, member | |
| member 14 | 14 (herpesvirus entry mediator), clone | |
| (herpesvirus entry | MGC: 3753, mRNA, complete cds. | |
| mediator) | /FEA: = mRNA /PROD = tumor necrosis factor | |
| SEQ ID NOS: 56 | receptor superfamily, member 14 (herpesvirus | |
| (DNA) and 168 | entry mediator) /DB_XREF = gi: 12803894 | |
| (amino acid) | /UG = Hs.279899 tumor necrosis factor | |
| | receptor superfamily, member 14 (herpesvirus | |
| | entry mediator) /FL = gb: BC002794.1 | |
| | gb: U70321.1 gb: U81232.1 gb: NM_003820.1 | |
| | gb: AF153978.1 | |
| tumor necrosis factor | gb: NM_000043.1 /DEF = Homo sapiens tumor | 204781_s_at |
| receptor superfamily, | necrosis factor receptor superfamily, member | |
| member 6 | 6 (TNFRSF6), mRNA. /FEA = mRNA | |
| SEQ ID NOS: 57 | /GEN = TNFRSF6 /PROD = apoptosis (APO-1) | |
| (DNA) and 169 | antigen 1 /DB_XREF = gi: 4507582 | |
| (amino acid) | /UG = Hs.82359 tumor necrosis factor receptor | |
| | superfamily, member 6 /FL = gb: M67454.1 | |
| | gb: NM_000043.1 | |
| zinc finger protein | gb: NM_003438.1 /DEF = Homo sapiens zinc | 207394_at |
| 137 (clone pHZ-30) | finger protein 137 (clone pHZ-30) (ZNF137), | |
| SEQ ID NOS: 58 | mRNA. /FEA = mRNA /GEN = ZNF137 | |
| (DNA) and 170 | /PROD = zinc finger protein 137 (clone pHZ- | |
| (amino acid) | 30) /DB_XREF = gi: 4507988 /UG = Hs.151689 | |
| | zinc finger protein 137 (clone pHZ-30) | |
| | /FL = gb: NM_003438.1 gb: U09414.1 | |
| hypothetical protein | Consensus includes gb: AI339568 /FEA = EST | 222727_s_at |
| FLJ22233 | /DB_XREF = gi: 4076495 | |
| SEQ ID NO: 59 | /DB_XREF = est: qk67e10.x1 | |
| (DNA) | /CLONE = IMAGE: 1874058 /UG = Hs.286194 | |
| | hypothetical protein FLJ22233 | |
| | /FL = gb: NM_024959.1 | |
| regenerating gene | gb: AY007243.1 /DEF = Homo sapiens | 223447_at |
| type IV | regenerating gene type IV mRNA, complete | |
| SEQ ID NOS: 60 | cds. /FEA = mRNA /PROD = regenerating gene | |
| (DNA) and 171 | type IV /DB_XREF = gi: 12621025 | |
| (amino acid) | /UG = Hs.105484 Homo sapiens regenerating | |
| | gene type IV mRNA, complete cds | |
| | /FL = gb: AY007243.1 | |
| Homo sapiens cDNA: | Consensus includes gb: AK025615.1 | 225285_at |
| FLJ21962 fis, clone | /DEF = Homo sapiens cDNA: FLJ21962 fis, | |
| HEP05564 | clone HEP05564. /FEA = mRNA | |
| SEQ ID NO: 61 | /DB_XREF = gi: 10438186 /UG = Hs.7567 | |
| (DNA) | Homo sapiens cDNA: FLJ21962 fis, clone | |
| | HEP05564 | |
| ESTs | Consensus includes gb: N37023 /FEA = EST | 225407_at |
| SEQ ID NO: 62 | /DB_XREF = gi: 1158165 | |
| (DNA) | /DB_XREF = est: yy40d03.s1 | |
| | /CLONE = IMAGE: 273701 /UG = Hs.235883 | |
| | ESTs | |
| phosphoprotein | Consensus includes gb: AK000680.1 | 225626_at |
| associated with | /DEF = Homo sapiens cDNA FLJ20673 fis, | |
| glycosphingolipid- | clone KAIA4464. /FEA = mRNA | |
| enriched | /DB_XREF = gi: 7020924 /UG = Hs.266175 | |
| microdomains | phosphoprotein associated with GEMs | |
| SEQ ID NOS: 63 | /FL = gb: AF240634.1 gb: NM_018440.1 | |
| (DNA) and 172 | | |
| (amino acid) | | |
| prostate cancer | Consensus includes gb: AA633076 /FEA = EST | 226167_at |
| associated protein 7 | /DB_XREF = gi: 2556490 | |
| SEQ ID NO: 64 | /DB_XREF = est: nq38a06.s1 | |
| (DNA) | /CLONE = IMAGE: 1146130 /UG = Hs.27495 | |
| | prostate cancer associated protein 7 | |
| Homo sapiens, | Consensus includes gb: AA524690 /FEA = EST | 226168_at |
| Similar to RIKEN | /DB_XREF = gi: 2265618 | |
| cDNA 1110060O18 | /DB_XREF = est: ng38e07.s1 | |
| gene, clone | /CLONE = IMAGE: 937092 /UG = Hs.294143 | |
| MGC: 17236 | ESTs, Weakly similar to predicted using | |
| IMAGE: 3864137, | Genefinder C. elegans | |
| mRNA, complete cds | | |
| SEQ ID NO: 65 | | |
| (DNA) | | |
| hypothetical protein | Consensus includes gb: BF111925 /FEA = EST | 226171_at |
| FLJ20209 | /DB_XREF = gi: 10941704 | |
| SEQ ID NO: 66 | /DB_XREF = est: 7138g05.x1 | |
| (DNA) | /CLONE = IMAGE: 3523784 /UG = Hs.3685 | |
| | hypothetical protein FLJ20209 | |
| Homo sapiens mRNA | Consensus includes gb: AA532640 /FEA = EST | 226484_at |
| for KIAA1190 | /DB_XREF = gi: 2276894 | |
| protein, partial cds | /DB_XREF = est: nj17c04.s1 | |
| SEQ ID NOS: 67 | /CLONE = IMAGE: 986598 /UG = Hs.206259 | |
| (DNA) and 173 | Homo sapiens mRNA for KIAA1190 protein, | |
| (amino acid) | partial cds | |
| KIAA1543 protein | Consensus includes gb: AB040976.1 | 226494_at |
| SEQ ID NOS: 68 | /DEF = Homo sapiens mRNA for KIAA1543 | |
| (DNA) and 174 | protein, partial cds. /FEA = mRNA | |
| (amino acid) | /GEN = KIAA1543 /PROD = KIAA1543 | |
| | protein /DB_XREF = gi: 7959352 | |
| | /UG: Hs.17686 KIAA1543 protein | |
| hypothetical protein | Consensus includes gb: AK002203.1 | 226992_at |
| MGC20702 | /DEF = Homo sapiens cDNA FLJ11341 fis, | |
| SEQ ID NO: 69 | clone PLACE1010786. /FEA = mRNA | |
| (DNA) | /DB_XREF = gi: 7023932 /UG = Hs.10260 | |
| | Homo sapiens cDNA FLJ11341 fis, clone | |
| | PLACE1010786 | |
| Homo sapiens cDNA | Consensus includes gb: AA129774 /FEA = EST | 227019_at |
| FLJ13137 fis, clone | /DB_XREF = gi: 1690185 | |
| NT2RP3003150 | /DB_XREF = est: zl16h09.s1 | |
| SEQ ID NO: 70 | /CLONE = IMAGE: 502145 /UG = Hs.288905 | |
| (DNA) | Homo sapiens cDNA FLJ13137 fis, clone | |
| | NT2RP3003150 | |
| hypothetical protein | Consensus includes gb: AW138767 | 227180_at |
| FLJ23563 | /FEA = EST /DB_XREF = gi: 6143085 | |
| SEQ ID NO: 71 | /DB_XREF = est: UI-H-BI1-aep-a-12-0-UI.s1 | |
| (DNA) | /CLONE = IMAGE: 2719799 /UG = Hs.274256 | |
| | hypothetical protein FLJ23563 | |
| ESTs | Consensus includes gb: AW264333 | 227320_at |
| SEQ ID NO: 72 | /FEA = EST /DB_XREF = gi: 6641075 | |
| (DNA) | /DB_XREF = est: xq98e01.x1 | |
| | /CLONE = IMAGE: 2758680 /UG = Hs.21835 | |
| | ESTs | |
| ESTs | Consensus includes gb: BF589359 /FEA = EST | 227354_at |
| SEQ ID NO: 73 | /DB_XREF = gi: 11681683 | |
| (DNA) | /DB_XREF = est: nab25d01.x1 | |
| | /CLONE = IMAGE: 3266737 /UG = Hs.13256 | |
| | ESTs | |
| Homo sapiens, | Consensus includes gb: AW001287 | 227676_at |
| Similar to RIKEN | /FEA = EST /DB_XREF = gi: 5848203 | |
| cDNA 1810037C20 | /DB_XREF = est: wu27e06.x1 | |
| gene, clone | /CLONE = IMAGE: 2521282 /UG = Hs.61265 | |
| MGC: 21481 | ESTs, Weakly similar to G786_HUMAN | |
| IMAGE: 3852062, | PROTEIN GS3786 H. sapiens | |
| mRNA, complete cds | | |
| SEQ ID NO: 74 | | |
| (DNA) | | |
| ESTs, Weakly similar | Consensus includes gb: AA557324 /FEA = EST | 227702_at |
| to JX0331 laurate | /DB_XREF = gi: 2327801 | |
| omega-hydroxylase | /DB_XREF = est: nl81a02.s1 | |
| [H. sapiens] | /CLONE = IMAGE: 1057034 /UG = Hs.26040 | |
| SEQ ID NO: 75 | ESTs, Weakly similar to fatty acid omega- | |
| (DNA) | hydroxylase H. sapiens | |
| Homo sapiens cDNA: | Consensus includes gb: T86159 /FEA = EST | 227724_at |
| FLJ22063 fis, clone | /DB_XREF = gi: 714511 | |
| HEP10326 | /DB_XREF = est: yd84h07.s1 | |
| SEQ ID NO: 76 | /CLONE = IMAGE: 114973 /UG = Hs.10450 | |
| (DNA) | Homo sapiens cDNA: FLJ22063 fis, clone | |
| | HEP10326 | |
| GalNAc alpha-2,6- | Consensus includes gb: Y11339.2 | 227725_at |
| sialyltransferase I, | /DEF = Homo sapiens mRNA for GalNAc | |
| long form | alpha-2,6-sialyltransferase I, long form. | |
| SEQ ID NOS: 77 | /FEA = mRNA /PROD = GalNAc alpha-2,6- | |
| (DNA) and 175 | sialyltransferase I /DB_XREF = gi: 7576275 | |
| (amino acid) | /UG = Hs.105352 GalNAc alpha-2,6- | |
| | sialyltransferase I, long form | |
| ESTs, Weakly similar | Consensus includes gb: AI827789 /FEA = EST | 228241_at |
| to JE0350 Anterior | /DB_XREF = gi: 5448449 | |
| gradient-2 | /DB_XREF = est: wf33a07.x1 | |
| [H. sapiens] | /CLONE = IMAGE: 2357364 /UG = Hs.100686 | |
| SEQ ID NO: 78 | ESTs, Weakly similar to JE0350 Anterior | |
| (DNA) | gradient-2 H. sapiens | |
| ESTs | Consensus includes gb: AI700341 /FEA = EST | 228653_at |
| SEQ ID NO: 79 | /DB_XREF = gi: 4988241 | |
| (DNA) | /DB_XREF = est: wd06e10.x1 | |
| | /CLONE = IMAGE: 2327370 /UG = Hs.110406 | |
| | ESTs | |
| ESTs | Consensus includes gb: BG494007 /FEA = EST | 228716_at |
| SEQ ID NO: 80 | /DB_XREF = gi: 13455521 | |
| (DNA) | /DB_XREF = est: 602542289F1 | |
| | /CLONE = IMAGE: 4673182 /UG = Hs.203213 | |
| | ESTs | |
| anterior gradient 2 | Consensus includes gb: AI922323 /FEA = EST | 228969_at |
| (Xenepus laevis) | /DB_XREF = gi: 5658287 | |
| homolog | /DB_XREF = est: wn90h03.x1 | |
| SEQ ID NO: 81 | /CLONE = IMAGE: 2453141 /UG = Hs.293380 | |
| (DNA) | ESTs | |
| Homo sapiens cDNA: | Consensus includes gb: AK026984.1 | 229021_at |
| FLJ23331 fis, clone | /DEF = Homo sapiens cDNA: FLJ23331 fis, | |
| HEP12664 | clone HEP12664 /FEA = mRNA | |
| SEQ ID NO: 82 | /DB_XREF = gi: 10439980 /UG = Hs.50742 | |
| (DNA) | Homo sapiens cDNA: FLJ23331 fis, clone | |
| | HEP12664 | |
| ESTs | Consensus includes gb: AI559300 /FEA = EST | 229331_at |
| SEQ ID NO: 83 | /DB_XREF = gi: 4509505 | |
| (DNA) | /DB_XREF = est: tq43d03.x1 | |
| | /CLONE = IMAGE: 2211557 /UG = Hs.294140 | |
| | ESTs | |
| hypothetical protein | Consensus includes gb: AI830823 /FEA = EST | 229439_s_at |
| SEQ ID NO: 84 | /DB_XREF = gi: 5451416 | |
| (DNA) | /DB_XREF = est: wj52b06.x1 | |
| | /CLONE = IMAGE: 2406419 /UG = Hs.95549 | |
| | hypothetical protein | |
| ESTs | Consensus includes gb: BF431989 /FEA = EST | 229657_at |
| SEQ ID NO: 85 | /DB_XREF = gi: 11444103 | |
| (DNA) | /DB_XREF = est: nab84a05.x1 | |
| | /CLONE = IMAGE: 3274280 /UG = Hs.203213 | |
| | ESTs | |
| ESTs | Consensus includes gb: BF589413 /FEA = EST | 229893_at |
| SEQ ID NO: 86 | /DB_XREF = gi: 11681737 | |
| (DNA) | /DB_XREF = est: nab26b11.x1 | |
| | /CLONE = IMAGE: 3267020 /UG = Hs.55501 | |
| | ESTs | |
| brain-specific protein | Consensus includes gb: BG055052 /FEA = EST | 230104_s_at |
| p25 alpha | /DB_XREF = gi: 12512386 | |
| SEQ ID NO: 87 | /DB_XREF = est: nac94g06.x1 | |
| (DNA) | /CLONE = IMAGE: 3441995 /UG = Hs.29353 | |
| | brain-specific protein p25 alpha | |
| ESTs, Weakly similar | Consensus includes gb: BF110588 /FEA = EST | 230645_at |
| to MMHUE4 | /DB_XREF = gi: 10940278 | |
| erythrocyte | /DB_XREF = est: 7n39e12.x1 | |
| membrane protein | /CLONE = IMAGE: 3567071 /UG = Hs.150478 | |
| 4.1, parent splice | ESTs, Weakly similar to KIAA0987 protein | |
| form [H. sapiens] | H. sapiens | |
| SEQ ID NO: 88 | | |
| (DNA) | | |
| ESTs | Consensus includes gb: BF592062 /FEA = EST | 230760_at |
| SEQ ID NO: 89 | /DB_XREF = gi: 11684386 | |
| (DNA) | /DB_XREF = est: 7n98h06.x1 | |
| | /CLONE = IMAGE: 3572962 /UG = Hs.233890 | |
| | ESTs | |
| hepatocyte nuclear | Consensus includes gb: AI032108 /FEA = EST | 230914_at |
| factor 4, alpha | /DB_XREF = gi: 3250320 | |
| SEQ ID NO: 90 | /DB_XREF = est: ow92d11.x1 | |
| (DNA) | /CLONE = IMAGE: 1654293 /UG = Hs.54424 | |
| | hepatocyte nuclear factor 4, alpha | |
| ESTs | Consensus includes gb: AW203959 | 230944_at |
| SEQ ID NO: 91 | /FEA = EST /DB_XREF = gi: 6503431 | |
| (DNA) | /DB_XREF = est: UI-H-BI1-aeu-b-12-0-UI.s1 | |
| | /CLONE = IMAGE: 2720590 /UG = Hs.149532 | |
| | ESTs | |
| ESTs | Consensus includes gb: AI139990 /FEA = EST | 231022_at |
| SEQ ID NO: 92 | /DB_XREF = gi: 3647447 | |
| (DNA) | /DB_XREF = est: qa47d03.x1 | |
| | /CLONE = IMAGE: 1689893 /UG = Hs.134586 | |
| | ESTs | |
| ESTs | Consensus includes gb: AI806131 /FEA = EST | 231148_at |
| SEQ ID NO: 93 | /DB_XREF = gi: 5392697 | |
| (DNA) | /DB_XREF = est: wf06c06.x1 | |
| | /CLONE = IMAGE: 2349802 /UG = Hs.99376 | |
| | ESTs | |
| hypothetical protein | Consensus includes gb: AB046810.1 | 232083_at |
| FLJ23045 | /DEF = Homo sapiens mRNA for KIAA1590 | |
| SEQ ID NO: 94 | protein, partial cds. /FEA = mRNA | |
| (DNA) | /GEN = KIAA1590 /PROD = KIAA1590 | |
| | protein /DB_XREF = gi: 10047254 | |
| | /UG = Hs.101774 hypothetical protein | |
| | FLJ23045 | |
| Homo sapiens cDNA: | Consensus includes gb: AK026404.1 | 232321_at |
| FLJ22751 fis, clone | /DEF = Homo sapiens cDNA: FLJ22751 fis, | |
| KAIA0483, highly | clone KAIA0483, highly similar to AF016692 | |
| similar to AF016692 | Homo sapiens small intestinal mucin (MUC3) | |
| Homo sapiens small | mRNA. /FEA = mRNA | |
| intestinal mucin | /DB_XREF = gi: 10439257 /UG = Hs.271819 | |
| (MUC3) mRNA | Homo sapiens cDNA: FLJ22751 fis, clone | |
| SEQ ID NO: 95 | KAIA0483, highly similar to AF016692 | |
| (DNA) | Homo sapiens small intestinal mucin (MUC3) | |
| | mRNA | |
| Homo sapiens PAC | Consensus includes gb: AC004908 | 232641_at |
| clone RP5-855D21 | /DEF = Homo sapiens PAC clone RP5- | |
| SEQ ID NOS: 96 | 855D21 /FEA = CDS_3 | |
| (DNA), 176 (amino | /DB_XREF = gi: 4156179 /UG = Hs.249181 | |
| acid), 177 (amino | Homo sapiens PAC clone RP5-855D21 | |
| acid), and 178 (amino | | |
| acid) | | |
| putative microtubule- | Consensus includes gb: AJ251708.1 | 234669_x_at |
| binding protein | /DEF = Homo sapiens partial mRNA for | |
| SEQ ID NO: 97 | putative microtubule-binding protein. | |
| (DNA) | /FEA = mRNA /PROD = putative microtubule- | |
| | binding protein /DB_XREF = gi: 6491740 | |
| | /UG = Hs.326544 putative microtubule-binding | |
| | protein | |
| ESTs | Consensus includes gb: AI741469 /FEA = EST | 234970_at |
| SEQ ID NO: 98 | /DB_XREF = gi: 5109757 | |
| (DNA) | /DB_XREF = est: wg11b01.x1 | |
| | /CLONE = IMAGE: 2364745 /UG = Hs.57787 | |
| | ESTs | |
| ESTs | Consensus includes gb: AI417897 /FEA = EST | 235444_at |
| SEQ ID NO: 99 | /DB_XREF = gi: 4261401 | |
| (DNA) | /DB_XREF = est: tg55b06.x1 | |
| | /CLONE = IMAGE: 2112659 /UG = Hs.235860 | |
| | ESTs | |
| ESTs | Consensus includes gb: AA827649 /FEA = EST | 235515_at |
| SEQ ID NO: 100 | /DB_XREF = gi: 2900090 | |
| (DNA) | /DB_XREF = est: od01a12.s1 | |
| | /CLONE = IMAGE: 1357918 /UG = Hs.105317 | |
| | ESTs | |
| ESTs | Consensus includes gb: AI493909 /FEA = EST | 235562_at |
| SEQ ID NO: 101 | /DB_XREF = gi: 4394912 | |
| (DNA) | /DB_XREF = est: qz94e02.x1 | |
| | /CLONE = IMAGE: 2042234 /UG = Hs.6131 | |
| | ESTs | |
| ESTs | Consensus includes gb: AV741130 /FEA = EST | 235651_at |
| SEQ ID NO: 102 | /DB_XREF = gi: 10858711 | |
| (DNA) | /DB_XREF = est: AV741130 | |
| | /CLONE = CBCATB06 /UG = Hs.173704 | |
| | ESTs, Moderately similar to ALU8_HUMAN | |
| | ALU SUBFAMILY SX SEQUENCE | |
| | CONTAMINATION WARNING ENTRY | |
| | H. sapiens | |
| ESTs, Weakly similar | Consensus includes gb: AI864053 /FEA = EST | 235678_at |
| to I38588 reverse | /DB_XREF = gi: 5528160 | |
| transcriptase homolog | /DB_XREF = est: wj55h10.x1 | |
| [H. sapiens] | /CLONE = IMAGE: 2406787 /UG = Hs.39972 | |
| SEQ ID NO: 103 | ESTs, Weakly similar to I38588 reverse | |
| (DNA) | transcriptase homolog H. sapiens | |
| ESTs | Consensus includes gb: AW339510 | 235866_at |
| SEQ ID NO: 104 | /FEA = EST /DB_XREF = gi: 6836136 | |
| (DNA) | /DB_XREF = est: xz91h08.x1 | |
| | /CLONE = IMAGE: 2871615 /UG = Hs.42722 | |
| | ESTs | |
| ESTs | Consensus includes gb: AI076192 /FEA = EST | 236422_at |
| SEQ ID NO: 105 | /DB_XREF = gi: 3405370 | |
| (DNA) | /DB_XREF = est: oz01g07.x1 | |
| | /CLONE = IMAGE: 1674108 /UG = Hs.131933 | |
| | ESTs | |
| ESTs | Consensus includes gb: AL044570 /FEA = EST | 236548_at |
| SEQ ID NO: 106 | /DB_XREF = gi: 5432785 | |
| (DNA) | /DB_XREF = est: DKFZp434L082_s1 | |
| | /CLONE = DKFZp434L082 /UG = Hs.147975 | |
| | ESTs | |
| ESTs | Consensus includes gb: AI968097 /FEA = EST | 237835_at |
| SEQ ID NO: 107 | /DB_XREF = gi: 5764915 | |
| (DNA) | /DB_XREF = est: wu13a12.x1 | |
| | /CLONE = IMAGE: 2516830 /UG = Hs.131360 | |
| | ESTs | |
| ESTs | Consensus includes gb: AI733801 /FEA = EST | 237923_at |
| SEQ ID NO: 108 | /DB_XREF = gi: 5054914 | |
| (DNA) | /DB_XREF = est: qk39c04.x5 | |
| | /CLONE = IMAGE: 1871334 /UG = Hs.146186 | |
| | ESTs | |
| ESTs | Consensus includes gb: BF594323 /FEA = EST | 238103_at |
| SEQ ID NO: 109 | /DB_XREF = gi: 11686647 | |
| (DNA) | /DB_XREF = est: 7h79g07.x1 | |
| | /CLONE = IMAGE: 3322236 /UG = Hs.158989 | |
| | ESTs | |
| Homo sapiens, clone | Consensus includes gb: T69015 /FEA = EST | 238422_at |
| MGC: 16402 | /DB_XREF = gi: 680163 | |
| IMAGE: 3940360, | /DB_XREF = est: yc31f04.s1 | |
| mRNA, complete cds | /CLONE = IMAGE: 82303 /UG = Hs.192728 | |
| SEQ ID NO: 110 | ESTs | |
| (DNA) | | |
| ESTs | Consensus includes gb: AA502384 /FEA = EST | 238956_at |
| SEQ ID NO: 111 | /DB_XREF = gi: 2237351 | |
| (DNA) | /DB_XREF = est: ne27f11.s1 | |
| | /CLONE = IMAGE: 898605 /UG = Hs.151529 | |
| | ESTs | |
| ESTs | Consensus includes gb: AI739241 /FEA = EST | 238984_at |
| SEQ ID NO: 112 | /DB_XREF = gi: 5101222 | |
| (DNA) | /DB_XREF = est: wi14h02.x1 | |
| | /CLONE = IMAGE: 2390259 /UG = Hs.171480 | |
| | ESTs | |
| ESTs | Consensus includes gb: AA088446 /FEA = EST | 239065_at |
| SEQ ID NO: 113 | /DB_XREF = gi: 1633958 | |
| (DNA) | /DB_XREF = est: zl89f04.s1 | |
| | /CLONE = IMAGE: 511807 /UG = Hs.170298 | |
| | ESTs | |
| ESTs | Consensus includes gb: AI493046 /FEA = EST | 239148_at |
| SEQ ID NO: 114 | /DB_XREF = gi: 4394049 | |
| (DNA) | /DB_XREF = est: qz49b04.x1 | |
| | /CLONE = IMAGE: 2030191 /UG = Hs.146133 | |
| | ESTs | |
| ESTs | Consensus includes gb: AI243098 /FEA = EST | 239966_at |
| SEQ ID NO: 115 | /DB_XREF = gi: 3838495 | |
| (DNA) | /DB_XREF = est: qh26e03.x1 | |
| | /CLONE = IMAGE: 1845820 /UG = Hs.178398 | |
| | ESTs | |
| ESTs, Weakly similar | Consensus includes gb: AI633523 /FEA = EST | 240106_at |
| to A49175 Motch B | /DB_XREF = gi: 4684853 | |
| protein - mouse | /DB_XREF = est: th68b11.x1 | |
| [M. musculus] | /CLONE = IMAGE: 2123805 /UG = Hs.44705 | |
| SEQ ID NO: 116 | ESTs | |
| (DNA) | | |
| ESTs | Consensus includes gb: AI300126 /FEA = EST | 240830_at |
| SEQ ID NO: 117 | /DB_XREF = gi: 3959472 | |
| (DNA) | /DB_XREF = est: qn54f02.x1 | |
| | /CLONE = IMAGE: 1902075 /UG = Hs.257858 | |
| | ESTs | |
| ESTs | Consensus includes gb: AI917390 /FEA = EST | 240964_at |
| SEQ ID NO: 118 | /DB_XREF = gi: 5637245 | |
| (DNA) | /DB_XREF = est: ts79a05.x1 | |
| | /CLONE = IMAGE: 2237456 /UG = Hs.99415 | |
| | ESTs | |
| betacellulin | Consensus includes gb: AI620677 /FEA = EST | 241412_at |
| SEQ ID NO: 119 | /DB_XREF = gi: 4629803 | |
| (DNA) | /DB_XREF = est: tu85e09.x1 | |
| | /CLONE = IMAGE: 2257864 /UG = Hs.154191 | |
| | ESTs | |
| ESTs | Consensus includes gb: H05025 /FEA = EST | 241874_at |
| SEQ ID NO: 120 | /DB_XREF = gi: 868577 | |
| (DNA) | /DB_XREF = est: yl74g12.s1 | |
| | /CLONE = IMAGE: 43864 /UG = Hs.323767 | |
| | ESTs | |
| ESTs | Consensus includes gb: AW024656 | 242358_at |
| SEQ ID NO: 121 | /FEA = EST /DB_XREF = gi: 5878186 | |
| (DNA) | /DB_XREF = est: wu78h05.x1 | |
| | /CLONE = IMAGE: 2526201 /UG = Hs.233382 | |
| | ESTs, Moderately similar to AF119917 62 | |
| | PRO2822 H. sapiens | |
| ESTs | Consensus includes gb: BF696216 /FEA = EST | 242626_at |
| SEQ ID NO: 122 | /DB_XREF = gi: 11981624 | |
| (DNA) | /DB_XREF = est: 602124536F1 | |
| | /CLONE = IMAGE: 4281632 /UG = Hs.188724 | |
| | ESTs | |
| ESTs | Consensus includes gb: N57929 /FEA = EST | 242978_x_at |
| SEQ ID NO: 123 | /DB_XREF = gi: 1201819 | |
| (DNA) | /DB_XREF = est: yv61e06.s1 | |
| | /CLONE = IMAGE: 247234 /UG = Hs.48100 | |
| | ESTs | |
| ESTs, Weakly similar | Consensus includes gb: AI457984 /FEA = EST | 243729_at |
| to ALU1_HUMAN | /DB_XREF = gi: 4312002 | |
| ALU SUBFAMILY J | /DB_XREF = est: tj66a04.x1 | |
| SEQUENCE | /CLONE = IMAGE: 2146446 /UG = Hs.165900 | |
| CONTAMINATION | ESTs, Weakly similar to ALUC_HUMAN | |
| WARNING ENTRY | !!!! ALU CLASS C WARNING ENTRY !!! | |
| [H. sapiens] | H. sapiens | |
| SEQ ID NO: 124 | | |
| (DNA) | | |
| ESTs | Consensus includes gb: AA581439 /FEA = EST | 244650_at |
| SEQ ID NO: 125 | /DB_XREF = gi: 2359211 | |
| (DNA) | /DB_XREF = est: nh13c10.s1 | |
| | /CLONE = IMAGE: 952242 /UG = Hs.152328 | |
| | ESTs | |

Biological Validation of Biomarker Candidates: Modulation of Expression by Treatment with Ligands for EGFR or by Treatment with Inhibitors for EGFR

To validate the significance of the biomarker candidates to predict the activity of the EGFR pathway and thereby the sensitivity of cancer cell to inhibition of EGFR by therapy, genes that would be regulated by the EGFR pathway were identified. Demonstration of that property for the EGFR biomarker candidates described above would add additional credibility as it would link these genes functionally to the EGFR pathway. Colon cancer and a lung cancer cell lines were treated with epidermal growth factor, in the absence of serum or, in the presence of serum with the EGFR modulator BMS-461453 or the EGFR modulator cetuximab (also known as C225, a chimeric monoclonal EGFR antibody). To identify genes induced by epidermal growth factor, serum starved cells were treated with 20 ng/ml EGF for 0.5, 6, and 18 hours. Control cells were treated with media alone. The expression profiling was performed, and data was analyzed using GeneChip® Expression Analysis software MAS 5.0 (Affymetrix, Santa Clara, Calif.).

Genes inhibited by EGFR antagonists were identified by treating cells in the presence of 10% serum with 0.5 uM of BMS-461453 or 1 ug/ml or 5 ug/ml of C225 for 6 and 24 hours. Cells exposed to 0.05% DMSO were used as the experimental control. Expression profiling was performed, and data were analyzed using GeneChip® Expression Analysis software MAS 5.0.

The gene expression of the inhibitor or EGFR treated cell lines was compared pair-wise to the untreated controls. Polynucleotides from the biomarker list, in which expression was increased two fold with EGFR exposure or decreased two fold with EGFR inhibitor treatment compared to the untreated controls, were considered to be modulated by EGFR. These biomarkers are provided in Table 4. Examples of the biomarkers include EphA1, B-cell translocation gene 2, prostaglandin-endoperoxide synthase 2 and serine (or cysteine) proteinase inhibitor (clade B), which are highly expressed in sensitive cells and up regulated by treatment with EGFR. On the other hand, spondin 1, talin 2 and nuclear receptor subfamily 3 are genes whose expression levels correlate with sensitivity or resistance of colon cancer cell lines and are consistently down regulated by treatment with EGFR inhibitors BMS-461453 and C225. It appears that these biomarkers are likely to be directly or indirectly involved in the EGFR signaling pathway, based on their expression modulation by EGF or EGFR inhibitor treatment

Identification of Top Biomarkers

In an attempt to further prioritize biomarkers for use in predicting response of cancer cells to treatment with one or more EGFR modulators, the following filter criteria were used on the Table 4 biomarkers to identity a total of fourteen biomarkers (Table 5) as the top biomarkers:

(1) results from the highly significant correlation of gene expression with IC₅₀: A p-value<0.01 in the student TTEST or a Pearson value<−0.6 described above;

(2) results from the modulation of expression by EGFR ligand and/or EGFR inhibitor treatment described above; and

(3) biomarkers supported by literature revealing a direct relationship between the EGFR pathway and the biomarkers.

TABLE 5
Top Fourteen Biomarkers

| | Literature Support | Induced by EGF/ |
|---|---|---|
| Biomarker Name | Citation | Inhibited by EGFR antagonist |
| mucin 2, | J Biol Chem. 2002 Aug | Expression inhibited 2 fold by EGFR |
| intestinal/tracheal | 30; 277(35): 32258-67 | antagonist in GEO colon cancer cell |
| (MUC2) | | line |
| intestinal mucin 3 | No | Expression inhibited 2 fold by EGFR |
| (MUC3) | | antagonist in GEO colon cancer cell |
| | | line |
| Homo sapiens cystic | No | Expression stimulated 2 fold by |
| fibrosis | | EGFR in H292 lung cancer cell line |
| transmembrane | | |
| conductance | | |
| regulator | | |
| ATP-binding | | |
| cassette (sub-family | | |
| C, member 7) | | |
| (CFTR) | | |
| f-spondin | No | Expression inhibited 2 fold by EGFR |
| (KIAA0762) protein | | antagonist in LOVO colon cancer cell |
| | | line |
| 3-hydroxy-3- | J Invest Dermatol. 2000 | Expression stimulated 3 fold by |
| methylglutaryl- | Jan; 114(1): 83-7 | EGFR in H292 lung cancer cell line |
| Coenzyme A | | |
| synthase 2 | | |
| serine (or cysteine) | Electrophoresis. 2001 | Expression stimulated 2 fold by |
| proteinase inhibitor, | Aug; 22(14): 3001-8. | EGFR in H292 lung cancer cell line |
| clade B | | |
| (ovalbumin), | | |
| member 5 | | |
| (SERPINB5 | | |
| BTG family, | No | Expression stimulated 2 fold by |
| member 2 (BTG2) | | EGFR in H292 lung cancer cell line |
| talin 2 (TLN2) | No | Expression inhibited 2 fold by EGFR |
| | | antagonist in GEO colon cancer cell |
| | | line |
| arachidonic acid | J Biol Chem. 1994 Aug | no |
| epoxygenase | 26; 269(34): 21786-92. | |
| prostaglandin G/H | J Biol Chem. 1994 Aug | Expression stimulated 6 fold by |
| synthase and | 26; 269(34): 21786-92. | EGFR in H292 lung cancer cell line |
| cyclooxygenase | | |
| EphA1 (EPHA1) | No | Expression stimulated 2 fold by |
| | | EGFR in CACO2 colon cancer cell |
| | | line |
| hemoglobin, alpha 1 | No | Expression inhibited 2 fold by EGFR |
| (HBA1) | | antagonist in GEO colon cancer cell |
| | | line |
| bone morphogenetic | Development 2000 | no |
| protein 2 | Nov; 127(22): 4993-5005 | |
| betacellulin (BTC)* | Biochem Biophys Res | no |
| | Commun. 2002 Jun | |
| | 28; 294(5): 1040-6 | |

*The gene betacellulin showed counter regulation with EGFR expression as defined for the EGFR-A list but had just a p value of 0.04 in the Student's TTest for correlation with IC₅₀. It was still selected as a top biomarker for the strong literature support, as betacellulin is one of the published ligands of EGFR.

Utility of Biomarkers

Polynucleotides that correlate to a specific property of a biological system can be used to make predictions about that biological system and other biological systems. To show the predictive utility of biomarkers that correlate to EGFR modulator sensitivity and resistance, these polynucleotides were tested for their ability to predict the response of twenty two colon cancer cell lines to a small molecule EGFR modulator.

The invention includes single biomarkers including, for example, the fourteen top biomarkers which were tested in a voting scheme. For that purpose, the mean expression value was calculated for all fourteen biomarkers. Colon cancer cell lines which showed an expression level above the mean were then voted to be sensitive, and colon cancer cell lines with expression levels below the mean were voted to be resistant. After this procedure, the voting was compared to the actual sensitivity/resistance status according to the definition based on IC₅₀ (see above) and an error rate was calculated. The error rates of the fourteen top biomarkers are shown in Table 6.

TABLE 6
Error Rates of Fourteen Top Biomarkers

| | Pearsons | TTEST | Prediction |
|---|---|---|---|
| Biomarker Name | value | P value | error rate |
| mucin 2, | −0.531 | 0.0083 | 20% |
| intestinal/tracheal | | | |
| (MUC2) | | | |
| intestinal mucin 3 | −0.639 | 0.0004 | 11.72% |
| (MUC3) | | | |
| Homo sapiens cystic | −0.646 | 9E−05 | 5.9% |
| fibrosis | | | |
| transmembrane | | | |
| conductance | | | |
| regulator | | | |
| ATP-binding | | | |
| cassette (sub-family | | | |
| C, member 7) | | | |
| (CFTR) | | | |
| f-spondin | −0.622 | 0.0004 | 12.8% |
| (KIAA0762) protein | | | |
| 3-hydroxy-3- | −0.575 | 0.0029 | 21.75% |
| methylglutaryl- | | | |
| Coenzyme A | | | |
| synthase 2 | | | |
| serine (or cysteine) | −0.62 | 0.0028 | 21.75% |
| proteinase inhibitor, | | | |
| clade B | | | |
| (ovalbumin), | | | |
| member 5 | | | |
| (SERPINB5 | | | |
| BTG family, | −0.544 | 0.0042 | 20.5% |
| member 2 (BTG2) | | | |
| talin 2 (TLN2) | −0.874 | 3E−05 | 8.8% |
| EphA1 (EPHA1) | −0.647 | 0.0021 | 22% |
| hemoglobin, alpha 1 | −0.744 | 8E−05 | 20% |
| (HBA1) | | | |
| bone morphogenetic | −0.555 | 0.0091 | 31.8% |
| protein 2 | | | |
| betacellulin (BTC) | −0.536 | 0.047 | 43.5% |

The biomarkers talin, the Cystic fibrosis conductance regulator (CFTR), and mucin 3 were the best single biomarkers with error rates below 12%.

### EXAMPLES

### Example 1

### Methods

IC₅₀ Determination—In Vitro Cytotoxicity Assay

A small molecule EGFR inhibitor, erlotinib HCl (BMS-461453), was tested for cytoxicity in vitro against a panel of twenty-two human colon cancer cell lines available from the American Type Culture Collection. Cytotoxicity was assessed in cells by MTS (3-(4,5-dimethylthiazol-2-yl)-5-(3-carboxymethoxyphenyl)-2-(4-sulphenyl)-2H-tetrazolium, inner salt) assay (T. L. Riss et al., 1992, Mol. Biol. Cell, 3 (Suppl.): 184a).

To carry out the assays, the colon cells were plated at 4,000 cell/well in 96 well microtiter plates and 24 hours later serial diluted drugs were added. The concentration range for the EGFR inhibitor was from 5 μg/ml to 0.0016 μg/ml (roughly 10 μM to 0.0032 μM). The cells were incubated at 37° C. for 72 hours at which time the tetrazolium dye MTS (333 μg/ml final concentration) in combination with the electron coupling agent phenazine methosulfate (25 μM final concentration) was added. A dehydrogenase enzyme in live cells reduces the MTS to a form that absorbs light at 492 nm that can be quantified spectrophotometrically. The greater the absorbency, the greater the number of live cells. The results were expressed as an IC₅₀, which is the drug concentration required to inhibit cell proliferation (i.e., absorbance at 450 mm) to 50% of that of untreated control cells. The mean IC₅₀ and standard deviation (SD) from multiple tests for each cell line were calculated.

Resistant/Sensitive Classification

The cell lines with IC₅₀ below 6 μM were defined as sensitive to the EGFR inhibitor, whereas those with IC₅₀ above 6 μM were considered to be resistant. The resistant/sensitive classification are shown above in Table 1, with five cell lines classified as sensitive and seventeen cell lines classified as resistant.

Gene Expression Profiling

The colon cells were grown using standard cell culture conditions: RPMI 1640 supplemented to contain 10% fetal bovine serum, 100 IU/ml penicillin, 100 mg/ml streptomycin, 2 mM L-glutamine and 10 mM Hepes (all from GibcoBRL, Rockville, Md.). RNA was isolated from 50-70% confluent cells or drug-treated cells using the RNeasy™ kits commercially available from Qiagen (Valencia, Calif.). Quality of the RNA was checked by measuring the 28s:18s ribosomal RNA ratio using Agilent 2100 bioanalyzer (Agilent, Technologies, Rockville, Md.). Concentration of total RNA was determined spectrophotometrically. 10 μg of total RNA from each cell line was used to prepare biotinylated probe according to the Affymetrix Genechip® Expression Analysis Technical Manual, 2001. Targets were hybridized to Affymetrix high density oligonucleotide array human HG-U133 set chips (Affymetrix, Santa Clara, Calif.). Arrays were then washed, and stained using the GeneChip Fluidics station according to the manufacture's instructions. The HG-U133 set consisting of two GeneChip® arrays contains nearly 45,000 probe sets representing more than 39,000 transcripts derived from approximately 33,000 well-substantiated human genes.

Preprocessing of Microarray Data for Selecting Biomarkers

Scanned image files were visually inspected for artifacts and analyzed with GeneChip® Expression Analysis software MAS 5.0 (Affymetrix, Santa Clara, Calif.). The “Detection Call” (see Affymetrix manual) was used to determine whether a transcript was detected within one sample, as well as the “Signal” (see Affymetrix Genechip® Expression Analysis Technical Manual, 2001) which measured the relative abundance of a transcript. The trimmed mean intensity for each chip was scaled to 1,500 (see Affymetrix manual) in order to account for any minor differences in global chip intensity, so that the overall expression level for each cell line is comparable. Affymetrix control sequences were removed prior to analysis.

Induction Studies of Colon and Breast Cell Lines with EGFR Inhibitors or EGFR Ligand and Selection of Genes Modulated by the Inductions

The five colon cell lines and one lung cell line indicated with asterisks in Table 1 were used in the drug induction study. Three of the colon cell lines express EGFR and are sensitive to the EGFR inhibitor BMS461453. The SW480 cell line, while expressing EGFR, is insensitive to the EGFR inhibitor, and the COLO320_DM does not express EGFR and is EGFR inhibitor resistant. The lung cancer cell line H292 expresses EGFR, but its sensitivity status is unknown. Cells were seeded in a 10 cm² culture plate with the medium described above and cultured for 24 hours.

For the EGF induction studies, the colon cell line CACO2 and the lung cancer H292 cell line were washed 2×PBS, and the media was changed to RPMI without serum. The next day the cells were treated with 20 ng/ml EGF, and eventually lysed for RNA isolation 0.5, 6 and 18 hours post treatment. Gene expression was profiled as described below.

EGFR inhibition studies were conducted on the colon cell lines GEO, CCD33-CO, SW480 and COLO320DM. The expression profiling was performed as described above and data was analyzed using GeneChip® Expression Analysis software MAS 5.0. The expression data of EGFR inhibitor treated cell lines were compared pair-wise to that of untreated same cell line. A change was considered significant if a two fold difference in expression was demonstrated between the treated and the untreated control. Analysis was done for all four cell lines to compare the gene expression with or without EGFR inhibitor treatment.

### Example 2

### RT-PCR Expression Profiling

RNA quantification was performed using the SYBR Green real-time PCR. The SYBR Green real-time PCR assay is one of the most precise methods for assaying the concentration of nucleic acid templates.

RNA can be prepared using standard methods, preferably, employing the RNeasy Kit commercially available from Qiagen (Valencia, Calif.). cDNA template for real-time PCR can be generated using the Superscript™ First Strand Synthesis system for RT-PCR. SYBR Green real-time PCR reactions are prepared as follows: the reaction mix contains 20 ng first strand cDNA; 50 nM Forward Primer; 50 nM Reverse Primer; 0.75×SYBR Green I (Sigma); 1×SYBR Green PCR Buffer (50 mMTris-HCl pH 8.3, 75 mM KCl); 10% DMSO; 3 mM MgCl₂; 300 μM each dATP, dGTP, dTTP, dCTP; 1 U Platinum® Taq DNA Polymerase High Fidelity (Cat# 11304-029; Life Technologies; Rockville, Md.). Real-time PCR is performed using an Applied Biosystems 5700 Sequence Detection System. Conditions are 95° C. for 10 minutes (denaturation and activation of Platinum® Taq DNA Polymerase), 40 cycles of PCR (95° C. for 15 seconds, 60° C. for 1 minute). PCR products are analyzed for uniform melting using an analysis algorithm built into the 5700 Sequence Detection System.

cDNA quantification used in the normalization of template quantity is performed using SYBR Green real-time PCR Expression of EGFR is normalized to GAPDH expression as described below.

The sequences for the GAPDH oligonucleotides used in the SYBR Green real-time PCR reactions are:

| | |
|---|---|
| GAPDH-F: 5′-AGCCGAGCCACATCGCT-3′ | (SEQ ID NO: 191) |
| GAPDH-R: 5′-GTGACCAGGCGCCCAATAC-3′ | (SEQ ID NO: 192) |

The sequences for the EGFR oligonucleotides used in the SYBR Green real-time PCR reactions are:

(SEQ ID NO: 193)

EGFR-F: 5′-GCGTCTCTTGCCGGAATGT-3′

(SEQ ID NO: 194)

EGFR-R: 5′-AGCCGAGGCAGGGAATGCGTG-3′

The Sequence Detection System generates a Ct (threshold cycle) value that is used to calculate a concentration for each input cDNA template. cDNA levels for each gene of interest are normalized to GAPDH cDNA levels to compensate for variations in total cDNA quantity in the input sample. This is done by generating GAPDH Ct values for each cell line. Ct values for the gene of interest and GAPDH are inserted into a modified version of the δδCt equation (Applied Biosystems Prism® 5700 Sequence Detection System User Manual) which is used to calculate a GAPDH normalized relative cDNA level for each specific cDNA. The δδCt equation is: relative quantity of nucleic acid template=2^{δδCt}=2^{(δCta-δCtb)}, where δCta=Ct target−Ct GAPDH, and δCtb=Ct reference−Ct GAPDH.

### Example 3

### Production of Antibodies Against the Biomarkers

Antibodies against the biomarkers can be prepared by a variety of methods. For example, cells expressing an biomarker polypeptide can be administered to an animal to induce the production of sera containing polyclonal antibodies directed to the expressed polypeptides. In one aspect, the biomarker protein is prepared and isolated or otherwise purified to render it substantially free of natural contaminants, using techniques commonly practiced in the art. Such a preparation is then introduced into an animal in order to produce polyclonal antisera of greater specific activity for the expressed and isolated polypeptide.

In one aspect, the antibodies of the invention are monoclonal antibodies (or protein binding fragments thereof). Cells expressing the biomarker polypeptide can be cultured in any suitable tissue culture medium, however, it is preferable to culture cells in Earle's modified Eagle's medium supplemented to contain 10% fetal bovine serum (inactivated at about 56° C.), and supplemented to contain about 10 g/l nonessential amino acids, about 1,00 U/ml penicillin, and about 100 μg/ml streptomycin.

The splenocytes of immunized (and boosted) mice can be extracted and fused with a suitable myeloma cell line. Any suitable myeloma cell line can be employed in accordance with the invention, however, it is preferable to employ the parent myeloma cell line (SP2/0), available from the ATCC. After fusion, the resulting hybridoma cells are selectively maintained in HAT medium, and then cloned by limiting dilution as described by Wands et al. (1981, Gastroenterology, 80:225-232). The hybridoma cells obtained through such a selection are then assayed to identify those cell clones that secrete antibodies capable of binding to the polypeptide immunogen, or a portion thereof.

Alternatively, additional antibodies capable of binding to the biomarker polypeptide can be produced in a two-step procedure using anti-idiotypic antibodies. Such a method makes use of the fact that antibodies are themselves antigens and, therefore, it is possible to obtain an antibody that binds to a second antibody. In accordance with this method, protein specific antibodies can be used to immunize an animal, preferably a mouse. The splenocytes of such an immunized animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce the formation of further protein-specific antibodies.

### Example 4

### Immunofluorescence Assays

The following immunofluorescence protocol may be used, for example, to verify EGFR biomarker protein expression on cells or, for example, to check for the presence of one or more antibodies that bind EGFR biomarkers expressed on the surface of cells. Briefly, Lab-Tek II chamber slides are coated overnight at 4° C. with 10 micrograms/milliliter (μg/ml) of bovine collagen Type II in DPBS containing calcium and magnesium (DPBS++). The slides are then washed twice with cold DPBS++ and seeded with 8000 CHO-CCR5 or CHO pC4 transfected cells in a total volume of 125 μl and incubated at 37° C. in the presence of 95% oxygen/5% carbon dioxide.

The culture medium is gently removed by aspiration and the adherent cells are washed twice with DPBS+ at ambient temperature. The slides are blocked with DPBS++ containing 0.2% BSA (blocker) at 0-4° C. for one hour. The blocking solution is gently removed by aspiration, and 125 μl of antibody containing solution (an antibody containing solution may be, for example, a hybridoma culture supernatant which is usually used undiluted, or serum/plasma which is usually diluted, e.g., a dilution of about 1/100 dilution). The slides are incubated for 1 hour at 0-4° C. Antibody solutions are then gently removed by aspiration and the cells are washed five times with 400 μl of ice cold blocking solution. Next, 125 μl of 1 μg/ml rhodamine labeled secondary antibody (e.g., anti-human IgG) in blocker solution is added to the cells. Again, cells are incubated for 1 hour at 0-4° C.

The secondary antibody solution is then gently removed by aspiration and the cells are washed three times with 400 μl of ice cold blocking solution, and five times with cold DPBS++. The cells are then fixed with 125 μl of 3.7% formaldehyde in DPBS++ for 15 minutes at ambient temperature. Thereafter, the cells are washed five times with 400 μl of DPBS++ at ambient temperature. Finally, the cells are mounted in 50% aqueous glycerol and viewed in a fluorescence microscope using rhodamine filters.

What is claimed is:

1. A method for identifying a mammal that will respond therapeutically to a method of treating cancer comprising administering an EGFR modulator, wherein the method comprises:
(a) measuring in the mammal the level of at least one biomarker selected from the biomarkers of Table 4;
(b) exposing the mammal to the EGFR modulator;
(c) following the exposing of step (b), measuring in the mammal the level of the at least one biomarker,
wherein a difference in the level of the at least one biomarker measured in step (c) compared to the level of the at least one biomarker measured in step (a) indicates that the mammal will respond therapeutically to said method of treating cancer.

2. The method of claim 1 wherein the at least one biomarker is selected from the biomarkers of Table 5.

3. The method of claim 1 wherein the method is an in vitro method, and wherein the at least one biomarker is measured in at least one mammalian biological sample from the mammal.

4. A method for identifying a mammal that will respond therapeutically to a method of treating cancer comprising administering an EGFR modulator, wherein the method comprises:
(a) exposing the mammal to the EGFR modulator;
(b) following the exposing of step (a), measuring in the mammal the level of the at least one biomarker selected from the biomarkers of Table 4,
wherein a difference in the level of the at least one biomarker measured in step (b), compared to the level of the biomarker in a mammal that has not been exposed to said EGFR modulator, indicates that the mammal will respond therapeutically to said method of treating cancer.
